(19) United States
(12) Patent Application Publication
(10) Pub. No.: US 2021/0315959 A1
(43) Pub. Date: Oct. 14, 2021

(54) ANTI-CANCER AND ANTI-PROLIFERATIVE COMPOSITIONS, AND METHODS FOR THEIR USE IN TREATING CANCER

(71) Applicant: Natreon, Inc., New Brunswick, NJ (US)

(72) Inventors: Sanyasi R. Kalidindi, Monroe, NJ (US); Jugnu Jain, Hyderabad (IN); Bhawana Gupta, Hyderabad (IN)

(73) Assignee: Natreon, Inc., New Brunswick, NJ (US)

(21) Appl. No.: 17/192,712

(22) Filed: Mar. 4, 2021

(30) Foreign Application Priority Data
Mar. 4, 2020 (IN) 202041009290

(51) Int. Cl. A61K 36/58; A61K 36/81; A61K 33/00; A61K 45/06; A61P 35/04

(52) U.S. Cl. CPC A61K 36/58; A61K 36/81; A61K 2236/331; A61K 45/06; A61P 35/04; A61K 33/00

(57) ABSTRACT

The present invention is directed to methods of treating cancer and inhibiting proliferation of cancer cells with compositions comprising extracts prepared from Terminalia chebula fruits (for instance AyuFlex®), Terminalia bellerica fruits (for instance Ayuric®), Phyllanthus emblica fruits (for instance Capros®), Withania somnifera roots and leaves (for instance Sensoril®), Shilajit (for instance PrimaVie®), Azadirachta indica leaves and twigs (for instance PhytoBGS®), and/or combinations thereof, including a trivalent chromium complex with extracts of Shilajit and P. emblica (e.g. Crominex-3+®). Combinations of the extracts with anti-cancer drugs, and related methods, are also described.

### CROSS-REFERENCE TO RELATED APPLICATIONS

This application claims priority from Indian Provisional Application No. 202041009290 (IN), filed Mar. 4, 2020, which is incorporated by reference herein in its entirety.

### FIELD OF THE INVENTION

The present invention relates to compositions and methods for treating cancer and/or inhibiting the proliferation of cancer cells and/or cancer-associated cells with compositions comprising Terminalia chebula (e.g. AyuFlex®), Terminalia bellerica (e.g. Ayuric®), Phyllanthus emblica (e.g. Capros®), Withania somnifera (e.g. Sensoril®), Shilajit (e.g. PrimaVie®), Azadirachta indica (e.g. PhytoBGS®), and combinations thereof, including for instance a trivalent chromium complex with extracts of Shilajit and P. emblica (e.g. Crominex-3+®). Combinations of the extracts, co-administration with anti-cancer drugs, and related methods are also described.

### BACKGROUND

Terminalia chebula has been extensively used in Ayurveda, Unani and Homoeopathic systems of medicine for improvement of different health conditions. T. chebula may be rich in tannoids and may contain a variety of other constituents. Chemical constituents isolated from T. chebula may vary considerably in type and/or concentration due to number of factors, e.g., ecological variation, soil variation, nutrient variation, as well as variations in the process of extraction.

Terminalia bellerica (a.k.a. belerica, belirica, bellirica) is grown widely throughout India, Sri Lanka, and South East Asia. T. bellerica has been used for centuries in Ayurveda and may contain several chemical constituents in common with T. chebula.

Phyllanthus emblica, the Indian gooseberry, is also widely used in Indian medicine for treatment of various diseases.

Withania somnifera, commonly known as Ashwagandha, has been used in herbal formulations of the Ayurvedic or Indian system of medicine, for instance to help to ward off stress and act as an adaptogen.

Shilajit, also known as “Moomiyo,” is found in high altitudes, for instance of the Himalayan Mountains, and is considered one of the “wonder medicines” of Ayurveda, the traditional Indian system of medicine dating back to 3500 B.C.E. Shilajit is physiologically active organic matter, composed of rock humus, rock minerals, and organic substances that have been compressed by layers of rock mixed with marine organisms and microbial metabolites. Shilajit oozes out of the rocks as a black mass in the Himalayas at higher altitudes ranging from 1000 to 5000 meters, as the rocks become warm during summer. Shilajit contains fulvic acids (“FAs”) as its main components, along with dibenzo-α-pyrones (“DBPs”) and DBP chromoproteins, humic acid, and more than forty (40) minerals. DBPs are also known as Urolithins (e.g. Urolithin A, Urolithin B).

Azadirachta indica, commonly known as neem, nitree, or Indian lilac, is a tree in the mahogany family Meliaceae. It is one of two species in the genus Azadirachta, and is native to the Indian subcontinent, i.e., India, Nepal, Pakistan, Bangladesh, Sri Lanka, and Maldives. It is typically grown in tropical and semi-tropical regions. Neem trees also grow in islands located in the southern part of Iran. Its fruits and seeds are the source of neem oil.

Products made from neem trees have been used in India for over two millennia for their medicinal properties. Neem products are considered a major component in Siddha medicine and Ayurvedic and Unani medicine and are particularly prescribed for skin diseases. Neem oil is also used to promote healthy hair, to improve liver function, detoxify the blood, and balance blood sugar levels. Neem leaves have also been used to treat skin diseases like eczema and psoriasis.

Primary patient-derived cancer cells (PDCs), cultured directly from tissue obtained from surgery in cancer patients, provide a physiologically relevant platform for testing an individual's cancer cells and cell type responses to a specific drug or composition. Information derived from PDC testing may be useful in personalized as well as generalized cancer patient treatments.

In addition, in-vitro studies using standardized cancer cell lines provide valuable information for animal and human treatments.

### SUMMARY OF THE INVENTION

The present invention is directed to a method of inhibiting the proliferation of cancer cells and/or cancer-associated cells comprising the steps of providing a composition comprising Terminalia chebula (for instance, AyuFlex®), Terminalia bellerica (for instance, Ayuric®), Phyllanthus emblica (for instance, Capros®), Withania somnifera (for instance, Sensoril®), Shilajit (for instance, PrimaVie®), and/or Azadirachta indica (for instance, PhytoBGS®), and combinations thereof, including for instance a trivalent chromium complex with extracts of Shilajit and P. emblica (e.g. Crominex-3+®), and applying the composition to cancer cells in an amount effective to cause an anti-proliferative effect. In an embodiment, cancer cells inhibited according to the present invention include glioma cells, breast cancer cells (ER/PR+ Her2 equivocal (“HR+”), ER/PR− Her2+(“Her2+”), and Triple Negative (“TN”)), chronic lymphocytic leukemia cells (“CLL”), acute myeloid leukemia cells (“AML”), small cell lung cancer cells, non-small lung cancer cells, colon cancer cells, pancreatic cancer cells, prostate cancer cells, and/or ovarian cancer cells.

Also, the present invention is directed to a method of treating cancer in a subject in need of such treatment, comprising the steps of providing a composition comprising Terminalia chebula (for instance, AyuFlex®), Terminalia bellerica (for instance, Ayuric®), Phyllanthus emblica (for instance, Capros®), Withania somnifera (for instance, Sensoril®), Shilajit (for instance, PrimaVie®), and/or Azadirachta indica (for instance, PhytoBGS®), and combinations thereof, including for instance a trivalent chromium complex with extracts of Shilajit and P. emblica (e.g. Crominex-3+®), and administering the composition to the subject in an amount effective to treat the cancer, for instance by slowing the progression or growth of the cancer, stopping the progression or growth of the cancer, shrinking the cancerous tumor, reducing the number of cancer cells and/or cancer-associated cells in the subject, and/or rendering the cancer almost undetectable or undetectable in the subject. In an embodiment, a cancer treated according to the present application includes glioma, breast cancer (including ER/PR+ Her2 equivocal, ER/PR− Her2+, and Triple Negative), chronic lymphocytic leukemia, acute myeloid leukemia, colon cancer, small-cell lung cancer, non-small cell lung cancer, pancreatic cancer, prostate cancer, and/or ovarian cancer. In an embodiment, the composition is an extract, for instance a standardized aqueous extract, of the plants identified above. In an embodiment, a method of treatment according to this invention includes administering compositions such as extracts of the above with anti-cancer drugs to enhance the effects of the drugs and, in an embodiment, reduce side, adverse, or toxic effects of the drugs, in an embodiment by reducing the necessary dose of the traditional drug.

### BRIEF DESCRIPTION OF THE DRAWINGS

FIG. 1 is a graph illustrating anti-proliferative and anti-cancer effects of AyuFlex® on cancer PDCs (left to right: Glioma, Breast Cancer (HR+), Breast Cancer (Her2+), Breast Cancer (TN), CLL, AML) in an anti-proliferation assay (“% INHIBITION” marked at 0, 25, 50, 75, 100%, with extract concentrations shown in 3 concentrations for each cancer type, left to right: 100 ug/ml, 30 ug/ml, 10 ug/ml).

FIG. 2 is a graph illustrating anti-proliferative and anti-cancer effects of Ayuric® on cancer PDCs (left to right: Glioma, Breast Cancer (HR+), Breast Cancer (Her2+), Breast Cancer (TN), CLL, AML) in an anti-proliferation assay (“% INHIBITION” marked at 0, 25, 50, 75, 100%, with extract concentrations shown in 3 concentrations for each cancer type, left to right: 100 ug/ml, 30 ug/ml, 10 ug/ml).

FIG. 3 is a graph illustrating anti-proliferative and anti-cancer effects of Capros® on cancer PDCs (left to right: Glioma, Breast Cancer (HR+), Breast Cancer (Her2+), Breast Cancer (TN), CLL, AML) in an anti-proliferation assay (“% INHIBITION” marked at 0, 25, 50, 75, 100%, with extract concentrations shown in 3 concentrations for each cancer type, left to right: 100 ug/ml, 30 ug/ml, 10 ug/ml).

FIG. 4 is a graph illustrating anti-proliferative and anti-cancer effects of Sensoril® on cancer PDCs (left to right: Glioma, Breast Cancer (HR+), Breast Cancer (Her2+), Breast Cancer (TN), CLL, AML) in an anti-proliferation assay (“% INHIBITION” marked at 0, 25, 50, 75, 100%, with extract concentrations shown in 3 concentrations for each cancer type, left to right: 100 ug/ml, 30 ug/ml, 10 ug/ml).

FIG. 5 is a graph illustrating anti-proliferative and anti-cancer effects of PrimaVie® Shilajit on cancer PDCs (left to right: Glioma, Breast Cancer (HR+), Breast Cancer (Her2+), Breast Cancer (TN), CLL, AML) in an anti-proliferation assay (“% INHIBITION” marked at 0, 25, 50, 75, 100%, with extract concentrations shown in 3 concentrations for each cancer type, left to right: 100 ug/ml, 30 ug/ml, 10 ug/ml).

FIG. 6 is a graph illustrating anti-proliferative and anti-cancer effects of PhytoBGS® on cancer PDCs in an anti-proliferation assay (“% INHIBITION” marked at 0, 25, 50, 75, 100%, with extract concentrations shown in 3 concentrations for each cancer type, left to right: 100 ug/ml, 30 ug/ml, 10 ug/ml).

FIG. 7 is a graph illustrating anti-proliferative and anti-cancer effects of AyuFlex®, Ayuric®, Capros®, Sensoril®, PrimaVie®, and PhytoBGS® on glioma PDCs in an anti-proliferation assay.

FIG. 8 is a graph illustrating the efficacy of Ayuflex®, Capros®, and Ayuric® on glioma cancer cells in an anti-proliferation assay, alone or in combination with temozolomide, or in the combinations Ayuflex®+Capros®, Ayuflex®+Ayuric®, Capros®+Ayuric®.

FIG. 9 is a graph illustrating anti-proliferative and anti-cancer effects of AyuFlex®, Ayuric®, Capros®, Sensoril®, PrimaVie®, and PhytoBGS® on breast cancer “HR+” PDCs in an anti-proliferation assay.

FIG. 10 is a graph representing a dose-response curve of Ayuflex® on HR+ breast cancer cells.

FIG. 11 is a graph illustrating the efficacy of Ayuflex®, Capros®, and Ayuric® on HR+ breast cancer cells in an anti-proliferation assay, each in combination with docetaxel or 5-fluorouracil (5-FU), or in the combinations Ayuflex®+Capros®, Ayuflex®+Ayuric®, Capros®+Ayuric®.

FIG. 12 is a graph illustrating the efficacy of Ayuflex®, Capros®, and Ayuric® on HR+ breast cancer cells in an anti-proliferation assay, alone or in combination with docetaxel or 5-fluorouracil (5-FU), or in the combinations Ayuflex®+Capros®, Ayuflex®+Ayuric®, Capros®+Ayuric®, grouped by extract according to the invention.

FIG. 13 is a graph illustrating anti-proliferative and anti-cancer effects of AyuFlex®, Ayuric®, Capros®, Sensoril®, PrimaVie®, and PhytoBGS® on breast cancer “Her2+” PDCs in an anti-proliferation assay.

FIG. 14 is a graph representing a dose-response curve of Ayuflex® on Her2+ breast cancer cells.

FIG. 15 is a graph illustrating the efficacy of Ayuflex®, Capros®, and Ayuric® on Her2+ breast cancer cells in an anti-proliferation assay, each in combination with docetaxel or 5-fluorouracil, or in the combinations Ayuflex®+Capros®, Ayuflex®+Ayuric®, Capros®+Ayuric®.

FIG. 16 is a graph illustrating anti-proliferative and anti-cancer effects of AyuFlex®, Ayuric®, Capros®, Sensoril®, PrimaVie®, and PhytoBGS® on breast cancer “TN” (Triple Negative) PDCs in an anti-proliferation assay.

FIG. 17 is a graph representing a dose-response curve of Ayuflex® on TN breast cancer cells.

FIG. 18 is a graph illustrating the efficacy of Ayuflex®, Capros®, and Ayuric® on TN breast cancer cells in an anti-proliferation assay, each in combination with docetaxel or 5-fluorouracil, or in the combinations Ayuflex®+Capros®, Ayuflex®+Ayuric®, Capros®+Ayuric®.

FIG. 19 is a graph illustrating anti-proliferative and anti-cancer effects of AyuFlex®, Ayuric®, Capros®, Sensoril®, PrimaVie®, and PhytoBGS® on CLL (Chronic lymphocytic leukemia) PDCs in an anti-proliferation assay.

FIG. 20 is a graph representing a dose-response curve of Ayuflex®, Capros®, Ayuric®, and the drug ibrutinib on CLL cancer cells.

FIG. 21 is a graph illustrating the efficacy of Ayuflex®, Capros®, and Ayuric® on CLL cancer cells in an anti-proliferation assay, alone or in combination with ibrutinib, or in the combinations Ayuflex®+Capros®, Ayuflex®+Ayuric®, Capros®+Ayuric®.

FIG. 22 is a graph illustrating anti-proliferative and anti-cancer effects of AyuFlex®, Ayuric®, Capros®, Sensoril®, PrimaVie®, and PhytoBGS® on AML (Acute myeloid leukemia) PDCs in an anti-proliferation assay.

FIG. 23 is a graph representing a dose-response curve of PhytoBGS®, PrimaVie®, and Sensoril® on AML cancer cells.

FIG. 24 is a graph illustrating the efficacy of Phyto-BGS®, PrimaVie®, and Sensoril® on AML cancer cells in an anti-proliferation assay, alone or in combination with arsenic trioxide, cytarabine, or doxorubicin, or in the combinations PhytoBGS®+PrimaVie®, PhytoBGS®+Sensoril®, and PrimaVie®+Sensoril®.

FIG. 25 is a graph representing a dose-response curve of Sensoril® on AML subtype Acute Promyelocytic Leukemia (APL, APML) patient-derived cancer cells (PDCs), showing a 50% Inhibitory Concentration (IC₅₀) of 32.94 ug/ml.

FIG. 26 is a graph showing the inhibition of APL PDC proliferation with Sensoril® and cytarabine.

FIG. 27 is a graph showing the inhibition of APL PDC proliferation with Sensoril® alone and synergistically with Sensoril® and arsenic trioxide (As₂O₃) in combination.

FIG. 28 is a graph showing the inhibition of APL PDC proliferation with Sensoril® alone and synergistically with Sensoril® and doxorubicin in combination.

FIG. 29 is a graph showing the inhibition of non-small cell lung cancer cell proliferation with extracts of this invention.

FIG. 30 is a graph showing the inhibition of colon cancer cell proliferation with extracts of this invention.

FIG. 31 is a graph illustrating the efficacy of Ayuflex®, Capros®, and Ayuric® on glioma PDCs SB 32833 (Glioblastoma Grade IV) in an anti-proliferation assay, alone or in the combinations Ayuflex®+Capros®, Ayuflex®+Ayuric®, Capros®+Ayuric®, with GDC-0941 (10 uM), Doxorubicin (10 uM), and Temozolomide (100 uM) as controls.

FIG. 32 is a graph illustrating the efficacy of Ayuflex®, Capros®, and Ayuric® on glioma SB 6129 (anaplastic astrocytoma Grade III) patient-derived cancer cells in an anti-proliferation assay, alone (left to right: AyuFlex, Capros, Ayuric, or in combination Ayuflex®+Capros®, Ayuflex®+Ayuric®, Capros®+Ayuric®, with GDC-0941 (10 uM), Doxorubicin (10 uM), and Temozolomide (100 uM) as controls.

FIG. 33 is a graph illustrating the efficacy of extracts of this invention in inhibiting proliferation of glioblastoma cells (U87-MG cell line).

FIG. 34 is a graph illustrating the efficacy of extracts of this invention in inhibiting proliferation of glioblastoma cells (U87-MG cell line).

FIG. 35 illustrates dose-response curves of inhibitory activity of Sensoril® (IC₅₀ 67 ug/ml), cytarabine (IC₅₀ 567 nM), As₂O₃ (IC₅₀ 2.3 uM), and doxorubicin (IC₅₀ 79 nM) on AML cells (HL60 cell line).

FIG. 36 is a graph illustrating the efficacy of extracts of this invention in inhibiting Triple Negative Breast Cancer cell proliferation (SB 30750 PDCs) after a 72-hour incubation period.

FIG. 37 is a graph illustrating the efficacy of extracts of this invention in inhibiting Triple Negative Breast Cancer cell proliferation (MDAMB-231 cell line) after a 72-hour incubation period.

FIG. 38 is a graph illustrating the efficacy of extracts of this invention in inhibiting Triple Negative Breast Cancer cell proliferation (MDAMB-231 cell line) after a 120-hour incubation period.

FIG. 39 is a graph comparing the efficacy of extracts of this invention in inhibiting MDAMB-231 Triple Negative Breast Cancer cell proliferation (MDAMB-231 cell line) after 72- and 120-hour incubation periods.

FIG. 40 is a graph illustrating the efficacy of AyuFlex®, Ayuric®, and other extracts of this invention in inhibiting proliferation of small cell lung cancer cells. Extract concentrations are, left to right, 100 ug/ml, 30 ug/ml, 10 ug/ml.

FIG. 41 is a graph illustrating the efficacy of Ayuric®, Ayuflex®, Capros®, and other extracts of this invention in inhibiting proliferation of prostate cancer cells. Extract concentrations are, left to right, 100 ug/ml, 30 ug/ml, 10 ug/ml.

FIG. 42 is a graph illustrating the efficacy of Ayuflex®, Ayuric®, Sensoril®, and other extracts of this invention in inhibiting proliferation of ovarian cancer cells. Extract concentrations are, left to right, 100 ug/ml, 30 ug/ml, 10 ug/ml.

FIG. 43 illustrates dose-response curves of inhibitory activity of 6 different samples of hydroethanolic extracts of Withania somnifera on AML HL60 cell line cancer cells, and provides an IC₅₀ for each sample.

FIG. 44 is a graph illustrating the efficacy of standardized aqueous and hydroethanolic extracts of Withania somnifera (Sensoril®), Ayuflex®, Ayuric®, and other extracts of this invention in inhibiting proliferation of histiocytic lymphoma cancer cells. Extract concentrations are, left to right, 100 ug/ml, 30 ug/ml, 10 ug/ml.

FIG. 45 is a graph illustrating the efficacy of standardized aqueous and hydroethanolic extracts of Withania somnifera (Sensoril®), Ayuflex®, and other extracts of this invention in inhibiting proliferation of pancreatic cancer cells. Extract concentrations are, left to right, 100 ug/ml, 30 ug/ml, 10 ug/ml.

### DETAILED DESCRIPTION OF THE INVENTION

The below definitions and discussion are intended to guide understanding but are not intended to be limiting with regard to other disclosures in this application. References to percentage (%) in compositions of the present invention refers to the % by weight of a given component to the total weight of the composition being discussed, also signified by “w/w”, unless stated otherwise.

In the present invention, “anti-cancer” generally refers to preventing, treating and/or otherwise halting cancer in a subject for instance by reducing viability of cancer cells and/or cancer-associated cells, including for instance slowing the progression or growth of the cancer, stopping the progression or growth of the cancer, shrinking the cancerous tumor, reducing the number of cancer cells and/or cancer-associated cells in the subject, and/or rendering the cancer almost undetectable or undetectable in the subject. In an embodiment, cancer in a subject is diagnosed by a medical provider. In an embodiment, cancer in a subject is not diagnosed by a medical provider.

In the present invention, “anti-proliferative” and the like refers to inhibiting the proliferation of cancer cells, including for instance inhibiting the proliferation of cancer cells in a subject or for instance inhibiting the proliferation of cancer cells such as PDCs in a cell culture outside of a subject's body, by rendering the cancer cells temporarily or permanently non-viable (not able to grow or develop) and metabolically inactive. Reference to cells “inhibited by” a composition of this invention, or other substance, is to the inhibition of proliferation of cancer cells by the composition, for instance as shown by the anti-proliferation assays described in the Examples below, unless stated otherwise. In an embodiment, inhibiting the proliferation of cancer cells refers to killing cancer cells. In an embodiment, the proliferation of cancer cells may be inhibited by a composition of the present invention by 1%, 10%, 20%, 30%, 40%, 50%, 60%, 70%, 80%, 90%, or 100%, or for instance by 1%-100%, or any range of numbers therein, such as for instance 33-37% or 62%-75%. In an embodiment, the number of viable cancer cells may be reduced by 1%, 10%, 20%, 30%, 40%, 50%, 60%, 70%, 80%, 90%, or 100%, by 1%-100%, or any range of numbers therein, such as for instance 2-8% or 79-93%. Known anti-cancer agents GDC-0941 (a class I phosphatidylinositol 3 kinase (PI3K) inhibitor; pictilisib) and doxorubicin are shown as anti-proliferative drug controls in Example I below, inhibiting the proliferation of cancer cells by about 50% to about 100%, depending e.g. on the sensitivity of the PDC type. An “anti-cancer drug” may be pictilisib, doxorubicin, temozolomide, docetaxel, 5-fluorouracil (5-FU), ibrutinib, arsenic trioxide, and/or cytarabine, and/or any other control or standard of care drug identified in this application. Comparisons of different concentrations of standardized aqueous extracts of the present invention with the inhibition of proliferation of PDCs by GDC-0941 and doxorubicin are also shown in the Figures.

In the present invention, “cancer cells” refers to cells of, or taken from or derived from, a cancerous source, such as a solid cancerous tumor or hematopoietic cancerous tissue or cells. An example of “cancer cells” of the present invention is a mixed culture of primary patient-derived cancer cells (PDC), taken directly from an individual human subject's cancerous tissue post-surgery. PDCs may include cells from any cancer, including for instance cells from a glioma, such as a glioblastoma Grade IV; cells from a breast cancer tumor, such as breast cancer cells that are ER/PR+ Her2 equivocal, ER/PR-Her2+, or triple negative; leukemia cells from a subject having chronic lymphocytic leukemia; and/or leukemia cells from a subject having acute myeloid leukemia (e.g. M 4 subtype), and so forth. See for instance Table 1. Also, cancer cells of this invention include cells of a standard cell line, such as the cell lines discussed in the below Examples. In an embodiment, cancer cells of the present invention are part of a tumor or other cancerous cell source in a mammalian subject, such as in a human body. In an embodiment, cancer cells of the present invention include characteristics attributed to cells of a cancerous tumor or malignancy or the like, for instance as known in the art.

In the present invention, “applying” (and the like) a composition of the present invention refers to making the composition physically available to the cancer cells, for instance by administering the extract to a subject having cancer so that the extract or at least its active components reach the cancer cells in the subject's body. Applying the extract in an “effective amount” to cancer cells refers to applying the extract in an amount that will inhibit proliferation of the cancer cells, for instance as described in Example I and other Examples below.

In the present invention, “administering”, “administration”, and the like refer to providing a composition of the present invention to a subject so that the composition (or components thereof) reaches the subject's bloodstream and/or tissues and thus reaches cancer cells, and acts on the cancer cells to slow and/or stop their proliferation and render them non-viable. Administration may be by the subject or by another. Administration to the subject may be oral, for instance in the form of a dietary supplement, and/or in a solid pharmaceutical dosage form, preferably in a discrete dose unit, such as a table, hard gelatin capsule, soft gelatin capsule, etc. Administration may also be through parenteral, intramuscular, transdermal, topical, sublingual, intravenous, and other physiologically acceptable routes.

In an embodiment, references to active components and the like throughout this application are not intended as being bound by theory, as it is the administration of the full composition of the present invention which is shown to provide the remarkable inhibition of cancer cells of the present invention.

“Co-administration” refers to administering two or more compositions such as extract(s) of the present invention, or one or more such compositions with another composition or with an anti-cancer drug. Such co-administration may be at different times, so long as both extract and anti-cancer drug are available in the bloodstream and/or tissues of the subject in an effective amount to slow and/or stop the proliferation of cancer cells and in an embodiment render them non-viable. Doses of anti-cancer drug may be per the normal standard of care or at the lower end or in a lower dose, for instance in view of a subject's intolerance of adverse effects from the medication.

In the present invention, “treating cancer” and the like refers to halting the progression of cancer in a subject and/or causing the cancer including e.g. cancer cells to decrease, shrink, and if possible become or remain undetectable in the subject, for instance by reducing the size of a cancer tumor and/or number of cancerous cells. In an embodiment, the number of viable cancer cells and/or size of the tumor may be reduced by 1%, 5%, 10%, 20%, 30%, 40%, 50%, 60%, 70%, 80%, 90%, or 100%, by 1%-100%, or any range of numbers therein. In an embodiment, cancer being treated according to this invention is diagnosed in a subject by a medical professional, such that the subject has been diagnosed as having cancer. In an embodiment, cancer being treated according to this invention has not been diagnosed in a subject by a medical professional, such that the subject has not been diagnosed as having cancer, however cancer cells exist in the subject.

In an embodiment, a “subject” according to this invention is a human; in another embodiment, the subject is a mouse, rat, dog, horse, or any mammal including for instance a mammal serving as a model for cancer research, or a human. In an embodiment, treating the cancer with an “effective amount” of a composition of this invention includes administering an amount effective to slow the progression or growth of the cancer, stop the progression or growth of the cancer, shrink the cancerous tumor, reduce the number of cancer cells in the subject, and/or render the cancer almost undetectable or undetectable in the subject, and/or act against the cancer for instance as discussed elsewhere in this application or in the state of the art. “Enhancing” the treatment of cancer refers to administering a composition of this invention to a subject undergoing treatment with a different anti-cancer drug, to improve treatment outcome, as discussed throughout this application.

### Compositions

A “composition” of the present invention comprises Terminalia chebula (e.g. AyuFlex®), Terminalia bellerica (e.g. Ayuric®), Phyllanthus emblica (e.g. Capros®), Withania somnifera (e.g. Sensoril®), Shilajit (e.g. PrimaVie®), Azadirachta indica (e.g. PhytoBGS®), or a combination thereof, including for instance a trivalent chromium complex with extracts of Shilajit and P. emblica (e.g. Crominex-3+®). In an embodiment, a composition consists essentially of, or consists of, one or more of the above.

In an embodiment, a composition of this invention is an extract of Terminalia chebula (e.g. AyuFlex®), Terminalia bellerica (e.g. Ayuric®), Phyllanthus emblica (e.g. Capros®), Withania somnifera (e.g. Sensoril®), Shilajit (e.g. PrimaVie®), Azadirachta indica (e.g. PhytoBGS®), or a combination thereof, including for instance a trivalent chromium complex with extracts of Shilajit and P. emblica (e.g. Crominex-3+®). In an embodiment, an extract of this invention refers to a natural substance prepared from Terminalia chebula fruit, Terminalia bellerica fruit, Phyllanthus emblica fruit, Withania somnifera roots and leaves, Shilajit, Azadirachta indica twigs and leaves, or a combination thereof, that has been disrupted from its natural state and treated with water or aqueous solution such as phosphate buffered saline (PBS), alcohol such as methanol or ethanol, or a hydroalcoholic mixture. In an embodiment a hydroalcoholic mixture may include alcohol and water together, for instance in a ratio of 9:1 to 1:9; in another embodiment, an extract may be prepared by first treating the plant or e.g. herbomineral material with one substance, such as ethanol, and then with a second substance, such as water or an aqueous solution. See for instance Example IX. In an embodiment, extracted substances may be pooled. In an embodiment, an extract of this invention is a standardized extract.

In an embodiment, an aqueous standardized extract of this invention is AyuFlex®, Ayuric®, Capros®, Sensoril®, PrimaVie®, and/or PhytoBGS®. A composition of the present invention may be a combination, for instance, a blend of one or more extracts such as AyuFlex®, Ayuric®, Capros®, Sensoril®, PrimaVie®, and/or PhytoBGS®, and/or a trivalent chromium complex such as Crominex+3®. In an embodiment Ayuflex® and Ayuric® are combined. In another embodiment, AyuFlex® and Capros® are combined. In an embodiment, Ayuric® and Capros® are combined. In an embodiment, AyuFlex®, Ayuric®, and Capros® are all combined in a composition of the present invention. In an embodiment, Sensoril® and PrimaVie® are combined. In an embodiment, Sensoril® and Phyto-BGS® are combined. In an embodiment, PrimaVie® and Phyto-BGS® are combined. In an embodiment, Sensoril®, PrimaVie®, and Phyto-BGS® are combined. Other combinations of the present extracts may be combined into a composition of the present invention. A composition of the present invention may comprise, consist essentially of, or consist of, any extract of the present invention or combination thereof.

In an embodiment, an extract such as an aqueous standardized extract is in powdered form and may be blended together with another extract or trivalent chromium complex such as Crominex+3® or another substance, for instance in powdered form, or other solid or form. In another embodiment, the extract(s) may be for instance blended or dissolved into a composition in liquid form, or combined in another form. A composition of the present invention may further comprise one or more excipients, additives, and/or other substances, including for instance microcrystalline cellulose, croscarmellose sodium, magnesium stearate, and/or silicon dioxide; and/or a suitable aqueous solution such as a buffer solution. A composition of the present invention may be formulated into nutraceutical or pharmaceutical dosage forms comprising for instance tablets, capsules, powders, liquids, chews, gummies, transdermals, injectables, dietary supplements, topical creams or gels, lozenges, pills, and so forth. In an embodiment, a composition of the present invention is a solution comprising an extract and applied to cancer cells as in the Examples below, or used to prepare the applied solutions.

In an embodiment, a composition comprising one or more standardized aqueous extracts of this invention is administered in an effective amount to a mammal, including a daily dose for a human being of at least 1-2000 mg of at least one extract, in an embodiment at least 50 mg, 100 mg, 250 mg, 500 mg, 750 mg, 1000 mg, 1500 mg, or 2000 mg; and optionally 1-20000 mg of each and any other extract included in the composition. In a mammal, a dose may be about the same as in a human, and may be adjusted per kilogram of weight of the mammal. A powdered blend of one or more extracts and optionally excipients or other substances such as fillers, disintegrants, flow enhancers, and lubricants, for instance, microcrystalline cellulose, croscarmellose sodium, silicon dioxide, and magnesium stearate, may be blended using standard powder blending techniques. In an embodiment, a composition of this invention may be marked as organic by an appropriate agency or organization.

Bioactive Components of Extracts of this Invention

AyuFlex® of this invention is a standardized aqueous extract of the fruits of Terminalia chebula plant, off white to brown color powder and soluble in water with astringent taste. It contains not less than 39% w/w low molecular weight hydrolysable tannins as bioactives with not less than 27% w/w chebulinic acid and chebulagic acid combined and not less than 12% w/w of other unindentified low molecular weight hydrolysable tannins. Ellagic acid and gallic acid are also present in the extract, for which the analytical results may be reported without any specification, but specifications for these bioactives may also be identified, for instance in an embodiment, each may be present in amounts of less than 10% of the extract or composition. In an embodiment, an aqueous extract of this invention, Ayuflex®, contains about 65-70% w/w low molecular weight hydrolysable tannins including about 45-50% w/w chebulinic acid and chebulagic acid combined. In an embodiment, said aqueous extract may have a measured amount of 8-9% each of gallic acid and ellagic acid. In an embodiment, a composition of this invention may be a preparation of Terminalia chebula fruit, dried and powdered and in an embodiment, standardized for pharmaceutical or nutraceutical usage. In an embodiment, an extract of this invention may be prepared from such a fruit preparation.

Terminalia bellerica (for instance, Ayuric®)—Ayuric® of this invention is a standardized aqueous extract of the fruits of Terminalia bellerica plant, brown color powder and soluble in water with astringent taste. It contains not less than 15% w/w of low molecular weight hydrolysable tannins as bioactives, including chebulinic acid and chebulagic acid and other unidentified low molecular weight hydrolysable tannins. Ellagic acid and gallic acid are also present in the extract, for which the analytical results are only reported without any specification, but specifications for these bioactives may also be identified. In an embodiment, Ayuric® is a composition of this invention and contains 33-38% w/w low molecular weight hydrolysable tannins, including chebulinic acid and chebulagic acid and other unidentified low molecular weight hydrolysable tannins, and in a further embodiment, also contains 6-8% w/w gallic acid and 0.5-2% w/w ellagic acid. In an embodiment, a composition of this invention may be a preparation of Terminalia bellerica fruit, dried and powdered and in an embodiment, standardized for pharmaceutical or nutraceutical usage. In an embodiment, an extract of this invention may be prepared from such a fruit preparation.

Phyllanthus emblica (for instance, Capros®)—Capros® of this invention is a standardized aqueous extract of the fruits of Phyllanthus emblica plant, light yellow color powder and soluble in water with astringent taste. It contains not less than 60% w/w of low molecular weight hydrolysable tannins, comprising of emblicanin-A, emblicanin-B, punigluconin and pedunculagin, and not more than 4% w/w of gallic acid. In an embodiment, Capros® as a composition of this invention contains about 75-80% w/w low molecular-weight hydrolysable tannins, and about 0.5-1.5% w/w gallic acid. In an embodiment, a composition of this invention may be a preparation of Phyllanthus emblica fruit, dried and powdered and in an embodiment, standardized for pharmaceutical or nutraceutical usage. In an embodiment, an extract of this invention may be prepared from such a fruit preparation.

Withania somnifera (for instance, Sensoril®)—Sensoril® of this invention is a standardized aqueous extract of the roots and leaves of Withania somnifera plant, brown color powder and soluble in water with bitter taste. It contains not less than 10% w/w of withanolide glycosides, less than 0.5% w/w of withanolide aglycones (as Withaferin-A) and not less than 32% w/w oligosaccharides as bioactives. Withanolides, such as withastromolide, withanolide A, withanolide B, 27-hydroxy withanone, withanone, etc. may also be present in this extract. In an embodiment, Sensoril® as a composition of this invention contains about 10-12% w/w of withanolide glycosides, about 0-0.5% w/w of withanolide aglycones (as Withaferin-A), and 35-40% w/w oligosaccharides.

In an embodiment of the present invention, an extract of Withania somnifera is made by using ethanol extraction followed by water extraction. This extract is referred to as Sensoril®-AWE (“Alcohol-Water-Extract”). Other alcohols, such as methanol, isopropyl alcohol, etc. may also be used as extraction solvents instead of ethanol. Such a hydro-alcoholic Sensoril®-AWE extract of this invention contains not less than 10% w/w withanolide glycosides, not less than 2%, preferably not less than 3.0% w/w withanolide aglycones (as Withaferin-A) and not less than 20% w/w oligosaccharides as bioactives. Withanolides, such as withastromolide, withanolide A, withanolide B, 27-hydroxy withanone, withanone, etc. may also be present in this extract. In an embodiment, a composition of this invention is a hydroethanolic Withania somnifera (Sensoril®-AWE) extract containing 12-20% w/w withanolide glycosides (including as mentioned elsewhere in this application ranges therein, such as 12% w/w, 13% w/w, and so on), 2-8% w/w withanolide aglycones (as Withaferin-A), and 24-35% w/w oligosaccharides.

Shilajit (for instance, PrimaVie®)—PrimaVie® of this invention is a standardized aqueous extract of Shilajit, an herbo-mineral exudate from the Himalayan, Altai and other mountains, brown color powder and soluble in water with earthy taste. It contains not less than 50% w/w of fulvic acid and not less than 10.3% w/w of free dibenzo-α-pyrones and dibenzo-α-pyrones conjugated with chromoproteins combined. Dibenzo-α-pyrones are also known as Urolithins, for example Urolithin A and Urolithin B. In an embodiment, PrimaVie® as a composition of this invention is a standardized aqueous extract containing 58-63% fulvic acids with a dibenzo pyrone core nucleus and 14-18% of free dibenzo-α-pyrones and dibenzo-α-pyrones conjugated with chromoproteins combined.

Azadirachta indica (for instance, PhytoBGS®)—PhytoBGS® of this invention is a standardized aqueous extract of the leaves and twigs of Azadirachta indica plant, brown color powder and soluble in water with bitter taste. It contains not less than 2% w/w of flavonoids (comprising of quercetin-3-O-glucoside, quercetin-3-O-rutinoside, apigenin rutionoside and other rutin derivatives) and not less than 5% w/w and up to 20% w/w of myoinositol monophosphate as bioactives, and is devoid of possibly toxic compounds such as azadirachtone, azadiradione, nimbolide, nimbin. In an embodiment, a PhytoBGS® composition of this invention contains about 3% w/w flavonoids and 6-8% w/w myoinositol monophosphate.

Trivalent chromium complex (for instance, Crominex-3+®)—Crominex-3+® of this invention is a complex of trivalent chromium chloride, Phyllanthus emblica extract and Shilajit extract. It is a light brown color powder, soluble in water with astringent taste. It contains not less than 2% w/w of trivalent chromium as the bioactive. In an embodiment, a composition of this invention is Crominex-3+® containing 2-3% of trivalent chromium.

The bioactive compositions of the extracts described above are only representative and may differ depending on the analytical method used, especially so with natural products, as natural products contain multiple bioactives for which reference standards are not readily available in the market.

### Extraction Methodology

In an embodiment, a method of manufacturing of a T. chebula fruit extract is described in U.S. Pat. No. 10,500,240, which is incorporated by reference herein for this purpose, to the extent allowed by law. The same method is used for manufacturing T. bellerica extract also.

In an embodiment, the extraction process of the current invention includes the steps of providing dried fruits of T. chebula or T. bellerica, de-seeding the fruits, pulverizing or grinding the pulp to a powder, extracting the pulp powder with an extraction solvent or solvent mixture, optionally, with heating, to provide a T. chebula or T. bellerica enriched liquid extract, optionally concentrating the liquid extract and drying the concentrated liquid extract to provide a hydrolyzable tannoid enriched T. chebula or T. bellerica extract powder. Aqueous solvent is preferred. A particularly preferred solvent is water. Useful extraction temperatures can range from about 25° C. (ambient) to about 90° C. Particularly useful extraction temperatures can range from about 25° C. to about 80° C.

In an embodiment, AyuFlex® and Ayuric® are prepared in keeping with the methods described above.

In another embodiment, ethanol or methanol or a hydro-alcoholic mixture may be used as the solvent system for extraction.

In an embodiment, useful extraction times in conjunction with maintaining useful temperatures can range from about 2 hours to about 16 hours. A particularly useful extraction time range at about 25°±5° C. is from about 12 hours to about 16 hours, and at a temperature of 40°±5° C. is from about 2 hours to about 6 hours. Length and temperature of extraction may be varied at atmospheric pressure (i.e., approx. 1 atm). It is contemplated that pressure can be varied in the extraction process, for example, by use of a commercial pressure reactor apparatus.

The extraction process can also include drying the liquid extract to a powder form. Suitable drying methods include spray drying, lyophilization (freeze drying), vacuum drying (with or without heating), evaporation (with or without heating), and concentration under vacuum. Once isolated or obtained, the hydrolyzable tannoid enriched T. chebula or T. bellerica extract powder may be processed by any suitable means, including grinding, milling, sieving, sizing, blending and the like. The obtained hydrolyzable tannoid enriched T. chebula or T. bellerica extract powder may be prepared in any suitable particle size or particle size range.

Process additives such as microcrystalline cellulose, starch, maltodextrin and the like as carrier materials, anti-adherents such as silicone dioxide, rice bran powder and the like, and preservatives such as sodium benzoate, methyl paraben, propyl paraben, natural preservatives and the like may be added during the extraction process or during the final blending of the dried extract powder.

In the case of Phyllanthus emblica extract, seeds are removed from the fresh fruits after washing, the juice is separated from the pulp by pressing and/or centrifugation and the juice is dried by spray drying or another suitable drying technique, such as microwave drying, freeze drying, etc. Additives, such as preservatives, such as sodium chloride and sodium benzoate, carrier materials such as maltodextrin, microcrystalline cellulose, anti-adherent such as silicon dioxide and rice bran powder may also be added, optionally.

In the cases of Withania somnifera, Shilajit and Azadirachta indica, the dried roots and/or leaves of the plant, the shilajit stone and the dried leaves and twigs respectively, are milled and subjected to extraction and drying. Extraction is done using water only as the solvent or an alcohol, preferably ethanol or methanol, followed by water. The liquid extract is then optionally concentrated by evaporation and then dried by spray drying, freeze drying, microwave drying or another suitable drying technique. Extraction temperatures may range from 40° C.±5° C. to 90° C.±5° C., preferably 60° C.±5° C. to 80° C.±5° C. Extraction times may vary from 1 hour to 12 hours, preferably from 2 hours to 6 hours. Several cycles of extraction may also be done. In an embodiment of Azadirachta indica, only leaves may be used as the starting raw material.

Trivalent chromium complex is made by dissolving trivalent chromium chloride in water, with or without heat, adding Phyllanthus emblica extract and mixing for a suitable length of time to form a complex, then adding shilajit and mixing for a suitable length of time, then adding a carrier material such as microcrystalline cellulose, maltodextrin, starch, etc. and mixing for a suitable length of time, followed by spray drying, freeze drying, microwave drying or another suitable drying method. The temperature of the complexation step may be from 30° C.±5° C. to 80° C.±5° C., preferably from 40° C.±5° C. to 60° C.±5° C.

In an embodiment, a “combination” of this invention refers to a mixture of an extract of the present invention with another extract, and/or a mixture of one or more extract(s) with a known anti-cancer drug, for instance those drugs used as controls and/or identified as standard of care drugs in the Examples. In an embodiment, a combination is in the same composition. In an embodiment, a composition of the present invention includes one or more extracts as described, and the combination of extract with an anti-cancer drug occurs in a subject's body. The term “combination” is not intended to be limiting in the context of this invention. Combination therapy refers to a regimen for administering an extract including a combination of extracts of this invention with an anti-cancer drug, for instance to improve treatment outcome, and/or to increase anti-cancer effects as compared with the extract(s) alone and the anti-cancer drug alone. In an embodiment, a combination according to the present invention provides a synergistic effect, greater than the effect achieved by its individual components. While references to synergy and synergistic effects may be made throughout this application, all instances of such may not be expressly pointed out.

A “dietary supplement” according to the present invention refers to a composition of this invention which is orally administered as an addition to a subject's diet, which is not a natural or conventional food, which when administered inhibits the proliferation of cancer cells in the subject's body. In an embodiment, the dietary supplement is administered daily; in an embodiment, the dietary supplement is administered daily for instance for at least 3 days, 5 days, 1 week, 1 day to 1 week, 1 week to 26 weeks, or chronically for at least 1 day to 3 months, 6 months, 9 months, or 1 year or more, or for another period of time according to the present invention. A dietary supplement may be formulated into various forms, as discussed throughout this application. In an embodiment, a dietary supplement of this invention comprises one or more extracts of this invention, such as one or more aqueous standardized extracts or one or more hydroalcoholic extracts of the present invention.

### EXAMPLES

The present invention may be further understood in connection with the below Examples and with embodiments described throughout this application. The following non-limiting Examples and embodiments described below and throughout this application are provided to illustrate the invention. Materials and methods used in Example I were also used with regard to the other Examples, unless indicated or expressly stated otherwise.

### Example I

Anti-proliferation assays were performed on 6 types of primary patient-derived cancer cells (PDCs) from 6 different human donors, as described in Table 1:

TABLE 1
PDCs from 6 different human donors

| | | | Donor |
|---|---|---|---|
| | | Cancer | Gender/Age |
| Cancer Category | PDC | Characteristics | (years) |
| Solid | Glioma | Glioblastoma | M/74 |
| | | Grade IV | |
| Solid | Breast Cancer | ER/PR+ Her2 | M/52 |
| | | equivocal | |
| | | (“HR+”) | |
| Solid | Breast Cancer | ER/PR− Her2+ | F/45 |
| | | (“Her2+”) | |
| Solid | Breast Cancer | Triple negative | F/42 |
| | | (“TN”) | |
| Hematopoietic | CLL (Chronic | — | M/77 |
| | lymphocytic | | |
| | leukemia) | | |
| Hematopoietic | AML (Acute | M4 subtype | F/76 |
| | myeloid | | |
| | leukemia) | | |

Tests were performed in 384-well plates (24×16), using 1250 to 10,000 cells/well depending on the size and proliferation rate of different PDCs, in 30 ul of culture media (DMEM/F12 with serum and growth factors), with control wells set aside on each plate for media alone (4 wells), untreated PDCs (4 wells), and for PDCs treated with 10 ul of a known anti-cancer drug as a control. For instance, 10 ul of 40 uM GDC-0941 (pictilisib)), or 40 uM doxorubicin (2 wells each), was used, for a final concentration of 10 uM each well. The outermost rows/columns of the plates were filled with PBS (pH 7.4) to avoid an edge-effect and were considered as blanks.

Standardized aqueous extracts identified in Table 2 were prepared in main stock solutions by dissolving 1 mg of extract in 1 ml PBS (phosphate-buffered saline, pH 7.4).

TABLE 2
Examples of standardized aqueous extracts

| Preparation type | Source | Example |
|---|---|---|
| Standardized | Terminalia chebula fruits | AyuFlex ® |
| aqueous extract | | |
| Standardized | Terminalia bellerica fruits | Ayuric ® |
| aqueous extract | | |
| Standardized | Phyllanthus emblica fruits | Capros ® |
| aqueous extract | | |
| Standardized | Withania somnifera roots + leaves | Sensoril ® |
| aqueous extract | | |
| Standardized | Shilajit | PrimaVie ® |
| aqueous extract | | |
| Standardized | Azadirachta indica leaves + twigs | PhytoBGS ® |
| aqueous extract | | |

All extracts used in this Example were provided by Natreon, Inc. (New Brunswick, N.J.).

Anti-Proliferation Assay Protocol: 30 ul of cells were plated at the density of 1250 to 10,000 cells/well. The plate was incubated in a CO₂ incubator for 0/N incubation for 24 hours. Next day, 10 ul of 40 uM GDC-0941 (pictilisib) or 10 ul of 40 uM doxorubicin were added to incubated cells in the specified control drug wells, and 10 ul of 4× extract solutions were added to incubated cells in 3 concentrations: 400 ug/ml (2 wells), 120 ug/ml (3 wells), 40 ug/ml (3 wells). The final assay concentration of control drug was 10 uM for DGC-0941 (pictilisib) and doxorubicin. Final assay concentrations of made from stock solutions of standardized aqueous extracts identified in Table 2 were 100 ug/ml, 30 ug/ml, and 10 ug/ml. Since 10 ul extract was added to 30 ul of cell suspension, it diluted 4 times, so to maintain the final assay concentrations, 4× concentrated extract solutions (400 ug/ml, 120 ug/ml, and 40 ug/mo) were prepared from main stock (1 mg/ml) and added to the plate. In all experiments, 10 ul of 4 times concentrated stock (4×) was added to wells containing 30 ul of cell suspension to achieve final assay concentrations at 1X. Other anti-cancer drugs used as controls throughout this Example include temozolomide, docetaxel, 5-fluorouracil (5-FU), ibrutinib, arsenic trioxide, and cytarabine, and were applied to PDCs generally as described.

Once extracts were added to incubated cells and mixed gently, the plate was given a brief spin and incubated in the CO₂ incubator for 72 hours. The plate was removed from the incubator and 10 ul of detection reagent (CellTiterGlo®; Promega Corporation, Wisconsin, USA) added to each well of the 384 well plate. The plate was incubated (10 min) and luminescence counts measured, with luminescence produced proportional to the number of viable (living, metabolically active) cells for each assay. The assay described above is a two-dimensional (2D) assay.

References to synergy below are not intended as limiting; other instances of synergy may be present in view of the data provided below.

### Results and Discussion

1. Terminalia chebula Fruit Extract (AyuFlex®)

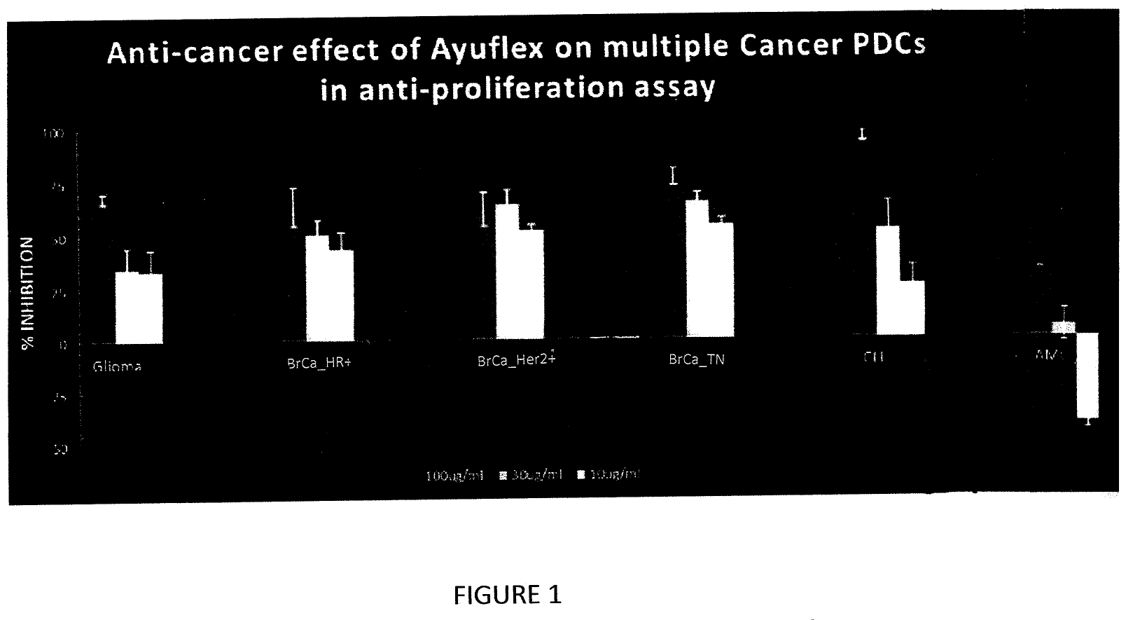

Table 3 shows the percentage of PDCs inhibited by AyuFlex® (±Standard Deviation) in the anti-proliferation assay described above. The results are pictured in FIG. 1. Bars shown from left to right for each cell type represent the application of 100 ug Ayuflex® extract/ml, 30 ug Ayuflex® extract/ml, and 10 ug Ayuflex® extract/ml to the cells.

TABLE 3
Inhibition of PDCs by AyuFlex ®

| | | Breast | Breast | Breast | | |
|---|---|---|---|---|---|---|
| % inhibition ±SD | Glioma | Cancer, HR+ | Cancer, Her2+ | Cancer, TN | CLL | AML |
| 100 ug/ml | 68 ± 2 | 64 ± 9 | 62 ± 8 | 77 ± 4 | 95 ± 2 | 33 ± 0 |
| 30 ug/ml | 35 ± 9 | 50 ± 7 | 64 ± 7 | 65 ± 4 | 52 ± 13 | 6 ± 8 |
| 10 ug/ml | 34 ± 10 | 43 ± 8 | 52 ± 3 | 54 ± 3 | 26 ± 9 | −41 ± 3 |

### Hematopoietic PDCs

AyuFlex® showed dose-dependent, high anti-cancer activity against CLL (Chronic lymphocytic leukemia) primary cells, with 50% of CLL cells inhibited by AyuFlex® extract at the 30 ug/ml concentration applied to the CLL cells, and 95% inhibition of CLL proliferation at the 100 ug/ml concentration. AyuFlex® inhibited about 33% of AML (acute myeloid leukemia) cells at the 100 ug/ml concentration.

### Solid Tumor PDCs

AyuFlex® showed potent anti-cancer activity against all solid cancer PDCs. The most potent anti-cancer activity was seen against Breast Cancer PDCs (Triple Negative (“TN”), and ER/PR− Her2+(“Her2+”), with AyuFlex® showing more than 50% inhibition of these cells at the concentration of 10 ug/ml. Anti-cancer activity against the other Breast Cancer PDCs (“HR+”) was also high, showing 50% inhibition at greater than/equal to the 30 ug/ml concentration. Anti-cancer activity against glioma cells were also high, with 35% inhibition at 30 ug/ml and 68% inhibition at the 100 ug/ml concentration.

2. Terminalia bellerica Fruit Extract (Ayuric®)

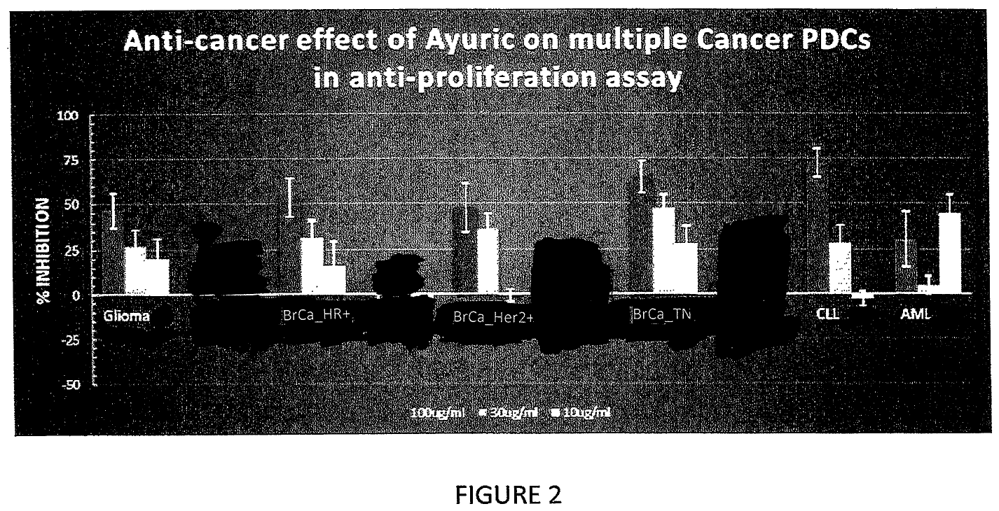

Table 4 shows the percentage of PDCs inhibited by Ayuric® (±Standard Deviation) in the anti-proliferation assay described above. The results are pictured in FIG. 2. Bars shown from left to right for each cell type represent the application of 100 ug Ayuric® extract/ml, 30 ug Ayuric® extract/ml, and 10 ug Ayuric® extract/ml to the cell wells.

TABLE 4
Inhibition of PDCs by Ayuric ®

| | | Breast | Breast | Breast | | |
|---|---|---|---|---|---|---|
| % inhibition ±SD | Glioma | Cancer, HR+ | Cancer, Her2+ | Cancer, TN | CLL | AML |
| 100 ug/ml | 47 ± 9 | 54 ± 11 | 48 ± 13 | 65 ± 9 | 73 ± 8 | 30 ± 15 |
| 30 ug/ml | 27 ± 9 | 32 ± 9 | 36 ± 8 | 47 ± 8 | 29 ± 10 | 5 ± 5 |
| 10 ug/ml | 20 ± 11 | 16 ± 13 | −1 ± 3 | 28 ± 10 | −3 ± 4 | 44 ± 11 |

### Hematopoietic Cancer PDCs

Ayuric® showed the highest anti-cancer activity against CLL primary cells, with dose-dependent inhibitory activity and 70% inhibition observed with the 100 ug/ml concentration applied to the CLL PDCs. In AML cells, Ayuric® exhibited moderate anti-cancer activity.

### Solid Tumor PDCs

Ayuric® showed good to moderate anti-cancer activity for all solid cancer PDCs. Ayuric® demonstrated dose-response inhibition and about 50% inhibition of breast cancer Triple Negative PDCs. The anti-cancer activity was found to be more moderate against HR+ breast cancer PDCs and glioma cells.

3. Phyllanthus emblica Fruit Extract (Capros®)

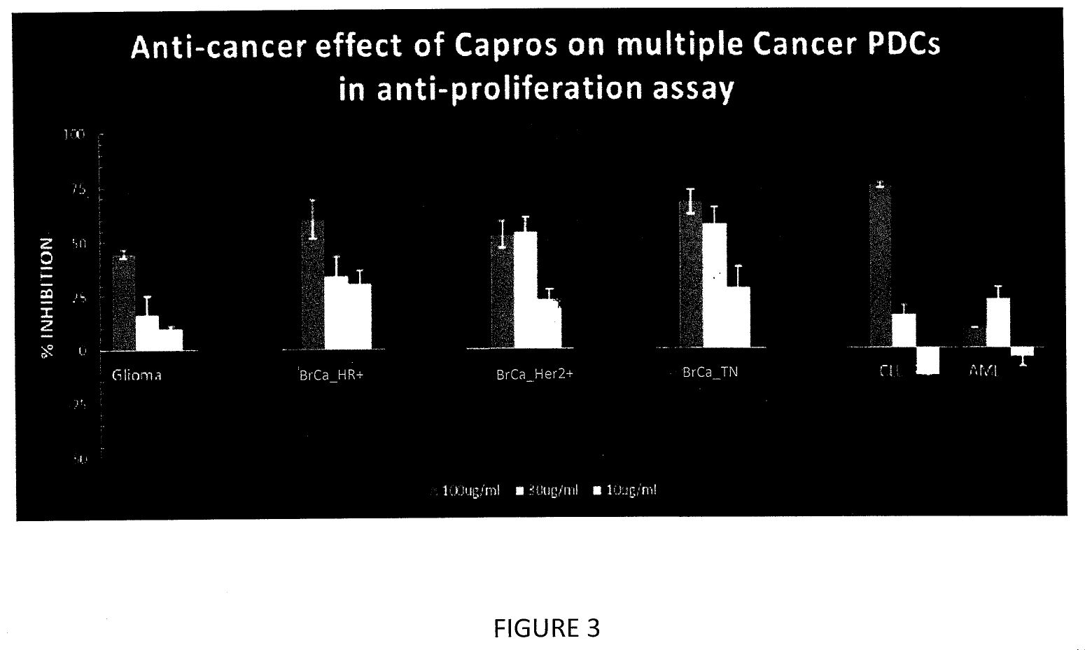

Table 5 shows the percentage of PDCs inhibited by Capros® (±Standard Deviation) in the anti-proliferation assay described above. The results are pictured in FIG. 3. Bars shown from left to right for each cell type represent the application of 100 ug Capros® extract/ml, 30 ug Capros® extract/ml, and 10 ug Capros® extract/ml to the cell wells.

TABLE 5
Inhibition of PDCs by Capros ®

| | | Breast | Breast | Breast | | |
|---|---|---|---|---|---|---|
| % inhibition ±SD | Glioma | Cancer, HR+ | Cancer, Her2+ | Cancer, TN | CLL | AML |
| 100 ug/ml | 45 ± 2 | 60 ± 9 | 53 ± 6 | 68 ± 6 | 75 ± 1 | 9 ± 0 |
| 30 ug/ml | 17 ± 8 | 34 ± 9 | 54 ± 7 | 58 ± 8 | 15 ± 4 | 23 ± 6 |
| 10 ug/ml | 10 ± 1 | 30 ± 6 | 23 ± 5 | 28 ± 10 | −13 ± 1 | −5 ± 4 |

### Hematopoietic PDCs

Capros® showed dose-response and highest anti-cancer activity against CLL primary cells, showing 75% inhibition at the 100 ug/ml concentration applied to the cells. In AML cells, Capros® inhibited about 25% of AML (acute myeloid leukemia) cells at the 30 ug/ml concentration.

### Solid Tumor PDCs

For all solid cancer PDCs, Capros® showed good to moderate anti-cancer activity. Capros® inhibited Breast Cancer Triple Negative and Her2+ cells by 50% at the 30 ug/ml concentration, and inhibited Glioma and Breast Cancer HR+ cells by 45% and 60% at the 100 ug/ml concentration.

4. Withania somnifera Roots+Leaves (Sensoril®), Aqueous Extract

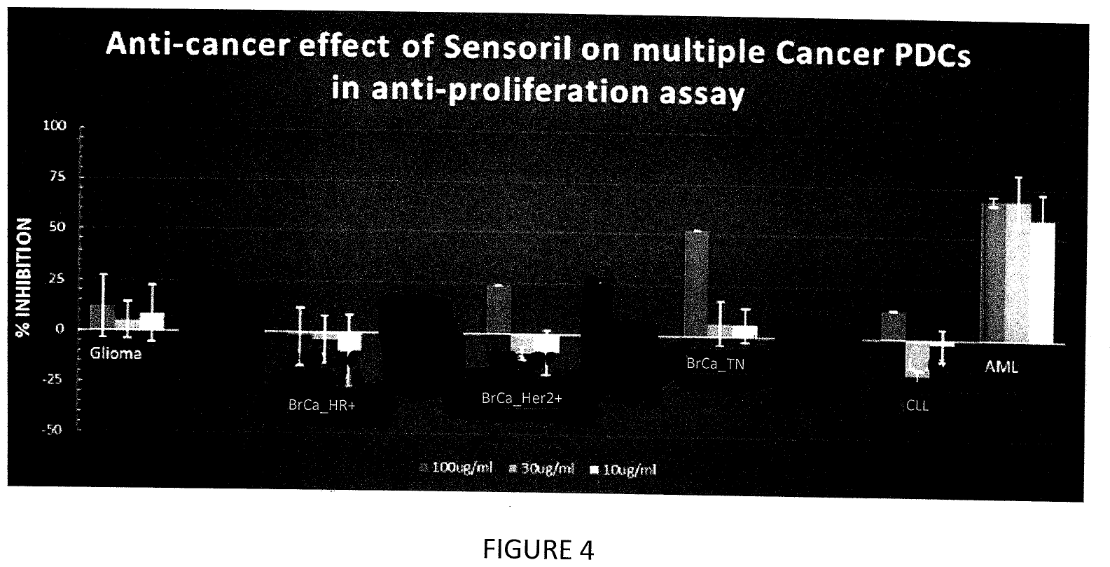

Table 6 shows the percentage of PDCs inhibited by Sensoril® (±Standard Deviation) in the anti-proliferation assay described above. The results are pictured in FIG. 4. Bars shown from left to right for each cell type represent the application of 100 ug Sensoril® extract/ml, 30 ug Sensoril® extract/ml, and 10 ug Sensoril® extract/ml to the cell wells.

TABLE 6
Inhibition of PDCs by Sensoril ®

| % inhibition ± | | Breast | Breast | Breast | | |
|---|---|---|---|---|---|---|
| SD | Glioma | Cancer HR+ | Cancer, Her2+ | Cancer TN | CLL | AML |
| 100 ug/ml | 12 ± 15 | −2 ± 14 | 24 ± 0 | 52 ± 0 | 14 ± 0 | 69 ± 2 |
| 30 ug/ml | 5 ± 9 | −4 ± 12 | −9 ± 3 | 7 ± 11 | −18 ± 3 | 69 ± 13 |
| 10 ug/ml | 8 ± 14 | −9 ± 18 | −9 ± 11 | 6 ± 8 | −3 ± 8 | 60 ± 13 |

### Hematopoietic PDCs

Sensoril® showed very potent activity against AML primary cells, inhibiting ≥50% at 10 ug/ml concentration. Sensoril® did not show much anti-cancer activity for CLL.

### Solid Tumor PDCs

Sensoril® inhibited the proliferation of breast cancer “TN” cells by 50% at the 100 ug/ml concentration. Sensoril® did not show substantial anti-cancer activity for glioma or the other breast cancer cells tested.

5. Shilajit (PrimaVie®)

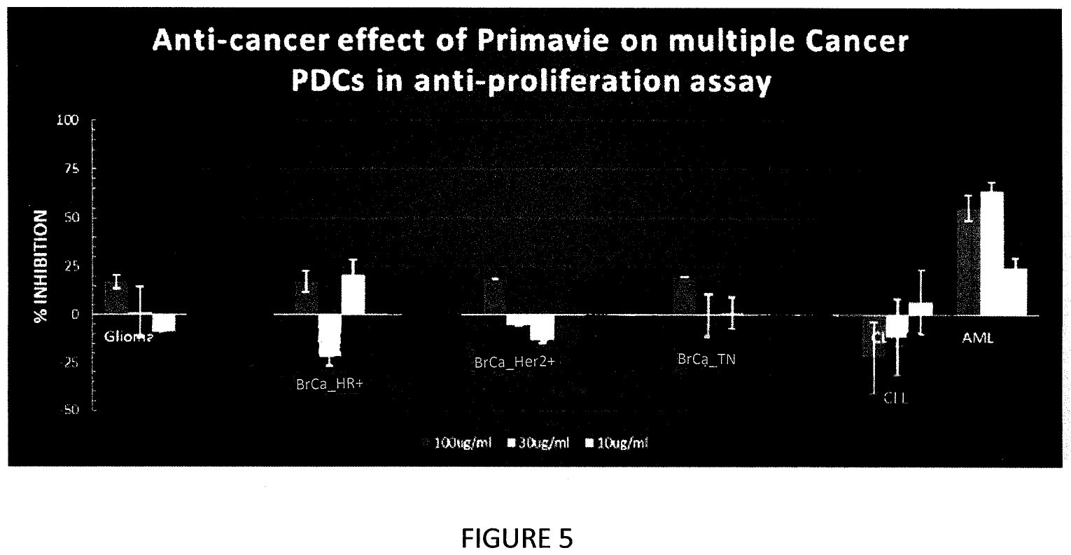

Table 7 shows the percentage of PDCs inhibited by PrimaVie® (±Standard Deviation) in the anti-proliferation assay described above. The results are pictured in FIG. 5. Bars shown from left to right for each cell type represent the application of 100 ug PrimaVie® extract/ml, 30 ug PrimaVie® extract/ml, and 10 ug PrimaVie® extract/ml to the cell wells.

TABLE 7
Inhibition of PDCs by PrimaVie ®

| | | Breast | Breast | Breast | | |
|---|---|---|---|---|---|---|
| % inhibition ±SD | Glioma | Cancer, HR+ | Cancer, Her2+ | Cancer, TN | CLL | AML |
| 100 ug/ml | 17 ± 4 | 17 ± 6 | 19 ± 0 | 20 ± 0 | −22 ± 19 | 56 ± 7 |
| 30 ug/ml | 1 ± 13 | −22 ± 4 | −5 ± 0 | 0 ± 11 | −12 ± 20 | 64 ± 4 |
| 10 ug/ml | −9 ± 5 | 21 ± 8 | −13 ± 2 | 1 ± 8 | 7 ± 17 | 25 ± 5 |

### Hematopoietic PDCs

PrimaVie® showed the highest anti-cancer activity against AML primary cells, inhibiting ≥50% at the 30 ug/ml concentration applied to the PDCs. PrimaVie® showed a lower anti-cancer activity for CLL PDCs.

### Solid Tumor PDCs

PrimaVie® showed a lower anti-cancer activity for all solid tumor PDCs.

6. Azadirachta indica Leaves+Twigs Extract (PhytoBGS®)

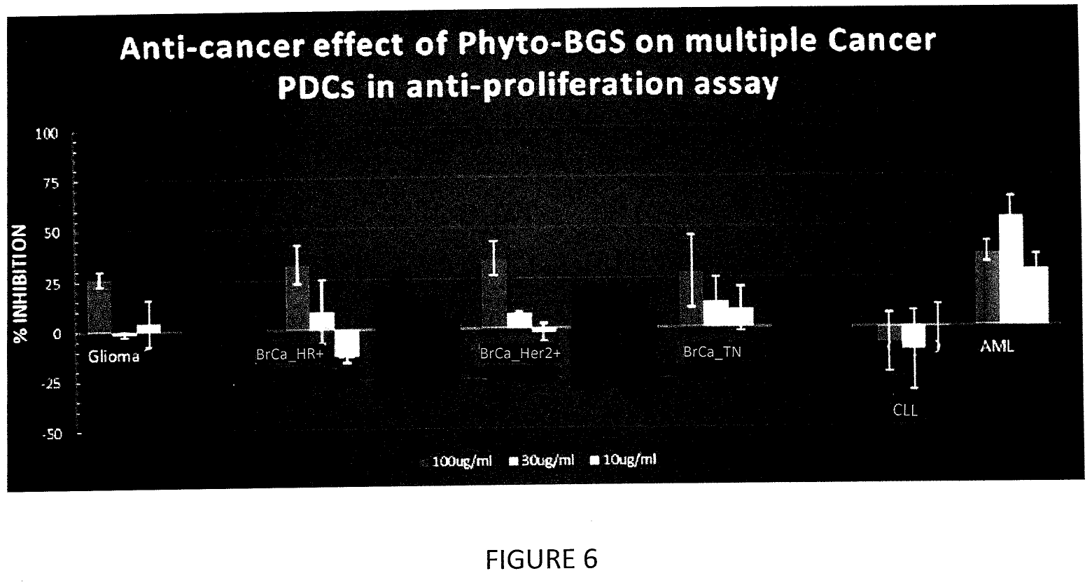

Table 8 shows the percentage of PDCs inhibited by Phyto-BGS® (±Standard Deviation) in the anti-proliferation assay described above. The results are pictured in FIG. 6. Bars shown from left to right for each cell type represent the application of 100 ug Phyto-BGS® extract/ml, 30 ug Phyto-BGS® extract/ml, and 10 ug Phyto-BGS® extract/ml to the cell wells.

TABLE 8
Inhibition of PDCs by Phyto-BGS ®

| | | Breast | Breast | Breast | | |
|---|---|---|---|---|---|---|
| % inhibition ±SD | Glioma | Cancer, HR+ | Cancer, Her2+ | Cancer, TN | CLL | AML |
| 100 ug/ml | 26 ± 4 | 32 ± 9 | 34 ± 8 | 27 ± 18 | −8 ± 15 | 37 ± 5 |
| 30 ug/ml | −1 ± 1 | 9 ± 16 | 7 ± 1 | 13 ± 12 | −12 ± 20 | 54 ± 10 |
| 10 ug/ml | 4 ± 12 | −13 ± 4 | −2 ± 4 | 9 ± 11 | −1 ± 11 | 28 ± 7 |

### Hematopoietic PDCs

PhytoBGS® showed the highest anti-cancer activity against AML primary cells, showing 50% inhibition at the 30 ug/ml concentration applied to the PDCs. PhytoBGS® showed a lower anti-cancer activity for CLL PDCs.

### Solid Tumor PDCs

PhytoBGS® showed a lower anti-cancer activity for all solid tumor PDCs.

### Further Results: Solid Cancer Cells, Hematopoietic Cancer Cells, Combination Therapies

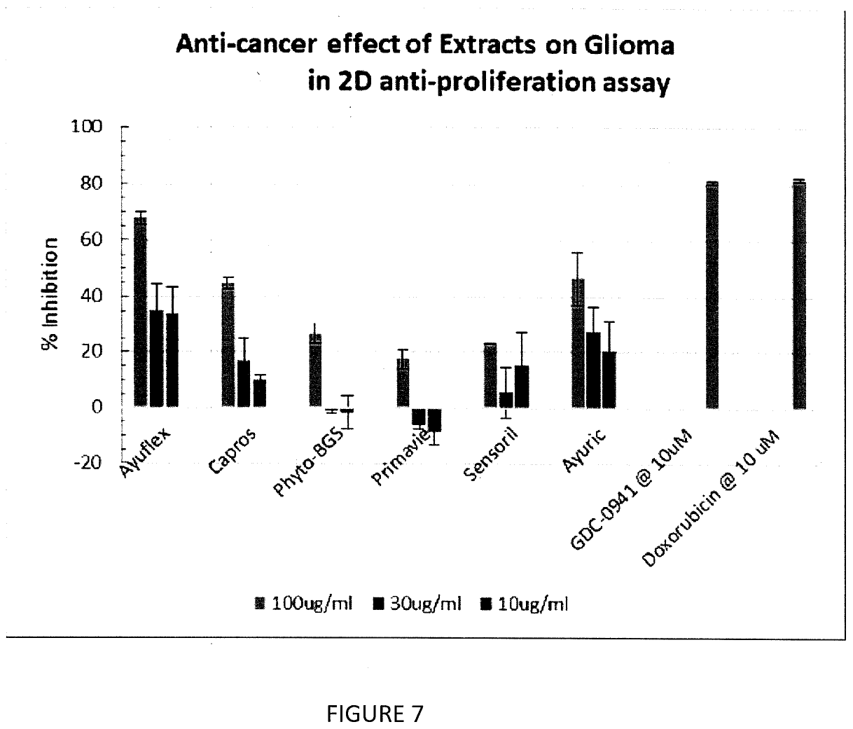

FIGS. 7, 9, 13, 16, 19, and 22 compare the anti-proliferative effect of the 6 different extracts tested on solid cancer cells and hematopoietic cancer cells. Bars shown from left to right for each extract represent the application of 100 ug extract/ml, 30 ug extract/ml, and 10 ug extract/ml to the cell wells.

FIG. 7 represents information provided in Tables 9-12B, showing a significant anti-cancer inhibition of proliferation of Glioma (Grade 4 Glioblastoma) cancer cells in the anti-proliferation assay. FIG. 7 compares the inhibition of proliferation of Glioma PDCs by the 6 different extracts in the assay, showing the highest inhibition by the application of known drugs 10 uM GDC-0941 (pictilisib) and 10 uM doxorubicin. Of the Extracts of the present invention, 100 ug/ml AyuFlex® showed 68% inhibition (±2, SD) of glioma PDC proliferation, as well as 35% (±9 SD) inhibition at the 30 ug/ml concentration and 34% inhibition (±10 SD) at the 10 ug/ml concentration. Capros® and Ayuric® at 100 ug/ml concentrations showed 47 and 45% inhibition of glioma PDC proliferation (±2 and 9, respectively). PhytoBGS®, PrimaVie®, and Sensoril® inhibited glioma PDCs proliferation at the highest concentration (100 ug/ml) at 26%±4, 17%±4, and 12%±15. 10 uM drug controls (GDC-0941 (pictilisib), doxorubicin) are shown at the far right of FIG. 7.

TABLE 9
Assay Controls

| | | | Assay | |
|---|---|---|---|---|
| | Avg | SD | fold | Z′ |
| 100% Proliferation (Cell Control) | 36996 | 5004 | 1298 | 0.59 |
| 0% Proliferation (Media only) | 29 | 22 | | |

TABLE 10
Assay Drug Control
GDC-0941 @ 10 uM

| Avg | | % Inhibition | | |
|---|---|---|---|---|
| n1 | n2 | n1 | n2 | Avg |
| 6290 | 7680 | 83 | 79 | 81 |

TABLE 11
Assay Drug Control
Doxorubicin @ 10 uM

| Avg | | % Inhibition | | |
|---|---|---|---|---|
| n1 | n2 | n1 | n2 | Avg |
| 6872 | 6345 | 81 | 83 | 82 |

TABLE 12A
Assay Results

| | % Inhibition | | | | | | | |
|---|---|---|---|---|---|---|---|---|
| | 100 ug/ml | | 30 ug/ml | | | 10 ug/ml | | |
| Ayuflex | 66 | 70 | 45 | 27 | 33 | 25 | 44 | 33 |
| Capros | 43 | 46 | 25 | 9 | 16 | 9 | 33* | 11 |
| Phyto-BGS | 24 | 29 | 22* | −2 | −1 | −7 | 16* | 2 |
| Primavie | 15 | 20 | 16* | −7 | −5 | −12 | 15* | −5 |
| Sensoril | 23 | 1* | 16 | 0 | 0 | −4 | 24 | 6 |
| Ayuric | 53 | 40 | 38 | 21 | 23 | 9 | 32 | 20 |

TABLE 12B
Assay Results

| | 100 ug/ml | | 30 ug/ml | | 10 ug/ml | |
|---|---|---|---|---|---|---|
| | Avg % | | Avg % | | Avg % | |
| | Inhibition | SD | Inhibition | SD | Inhibition | SD |
| AyuFlex ® | 68 | 2 | 35 | 9 | 34 | 10 |
| Capros ® | 45 | 2 | 17 | 8 | 10 | 1 |
| Phyto-BGS ® | 26 | 4 | −1 | 1 | −2 | 6 |
| PrimaVie ® | 17 | 4 | −6 | 2 | −9 | 5 |
| Sensoril ® | 23 | 0 | 5 | 9 | 15 | 12 |
| Ayuric ® | 47 | 9 | 27 | 9 | 20 | 11 |

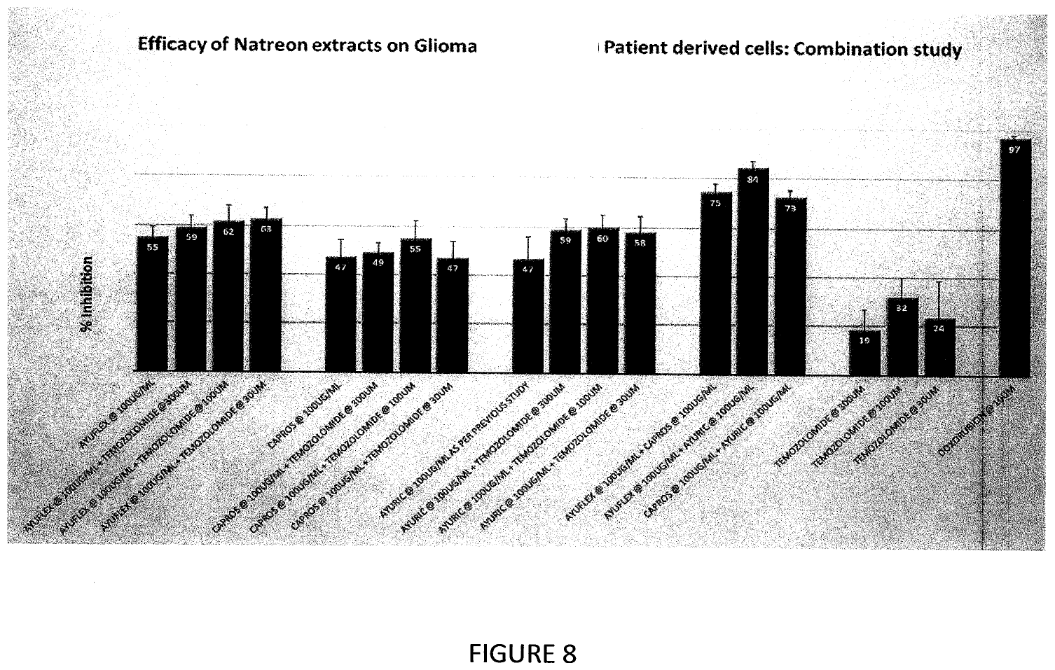

FIG. 8 represents information provided in Tables 13-16, showing a significant anti-cancer inhibition of glioma cell proliferation. Extracts Ayuric®, Capros®, and Ayuflex® inhibited proliferation of glioma (brain cancer) cells by about 50% or higher, alone, and also in combination with temozolomide, a current and popular drug used to treat glioma. As shown in FIG. 8, all three extracts—Ayuric®, Capros®, and Ayuflex®—showed better inhibition than temozolomide when tested alone or in combination. Temozolomide is only effective in about 20% of glioma patients. FIG. 8 shows that when combined with Ayuric®, Capros®, or Ayuflex®, the effectiveness of temozolomide is approximately doubled. The combination of temozolomide and Ayuric®, Capros®, or Ayuflex® appears to provide a synergistic inhibition of the proliferation of glioma cells.

Also, FIG. 8 shows the first application of Ayuflex® and Capros® combined; Ayuflex® and Ayuric® combined; and Capros® and Ayuric® combined on PDC glioma cells, showing 73% or higher inhibition of proliferation. The combinations of 100 ug/ml Ayuflex® and 100 ug/ml Capros® and of 100 ug/ml Capros® and 100 ug/ml Ayuric® resulted in about 75% and about 73% inhibition of glioma cell proliferation, respectively. The combination of 100 ug/ml Ayuflex® and 100 ug/ml Ayuric® resulted in about 84% inhibition of glioma cell proliferation. Doxorubicin is presented as a control drug at the far right of FIG. 8.

TABLE 13
PLATE MAP

| | | | |
|---|---|---|---|
| Cell | Ayuflex @ 100 ug/ml | Capros @ 100 ug/ml | Doxorubin @ 10 uM |
| Control | Ayuflex @ 100 ug/ml + | Ayuflex @ 100 ug/ml + | Ayuflex @ 100 ug/ml + |
| | Temozolomide @ 300 uM | Temozolomide @ 100 uM | Temozolomide @ 30 uM |
| | Capros @ 100 ug/ml + | Capros @ 100 ug/ml + | Capros @ 100 ug/ml + |
| | Temozolomide @ 300 uM | Temozolomide @ 100 uM | Temozolomide @ 30 uM |
| | Ayuric @ 100 ug/ml + | Ayuric @ 100 ug/ml + | Ayuric @ 100 ug/ml + |
| | Temozolomide @ 300 uM | Temozolomide @ 100 uM | Temozolomide @ 30 uM |
| Media | Ayuflex @ 100 ug/ml + | Ayuflex @ 100 ug/ml + | Capros @ 100 ug/ml + |
| | Capros @ 100 ug/ml | Ayuric @ 100 ug/ml | Ayuric @ 100 ug/ml |
| | Temozolomide @ 300 uM | Temozolomide @ 100 uM | Temozolomide @ 30 uM |

TABLE 14
PLATE QUALITY CONTROL
Analysis

| | | | Assay | |
|---|---|---|---|---|
| | Avg | SD | fold | Z′ |
| 100% Proliferation (Cell Control) | 62598 | 3313 | 4317 | 0.84 |
| 0% Proliferation (Media only) | 15 | 1 | | |

TABLE 15
DRUG CONTROL
Doxorubicin @ 10 uM

| | |
|---|---|
| Avg | % Inhibition |

| n1 | n2 | n3 | n1 | n2 | n3 | Avg |
|---|---|---|---|---|---|---|
| 1418 | 1584 | 2381 | 98 | 97 | 96 | 97 |

TABLE 16
RESULTS OF COMBINATION STUDY

| | Raw data | | | % Inhibition | | | | |
|---|---|---|---|---|---|---|---|---|
| Combination | n1 | n2 | n3 | n1 | n2 | n3 | Avg | SD |
| Ayuflex @ 100 ug/ml | 25994 | 31061 | 27155 | 58 | 50 | 57 | 55 | 4 |
| Ayuflex @ 100 ug/ml + | 23635 | 28706 | 24030 | 62 | 54 | 62 | 59 | 5 |
| Temozolomide @ 300 uM | | | | | | | | |
| Ayuflex @ 100 ug/ml + | 19216 | 26462 | 26112 | 69 | 58 | 58 | 62 | 7 |
| Temozolomide @ 100 uM | | | | | | | | |
| Ayuflex @ 100 ug/ml + | 20081 | 25499 | 23823 | 68 | 59 | 62 | 63 | 4 |
| Temozolomide @ 30 uM | | | | | | | | |
| Capros @ 100 ug/ml | 27974 | 34706 | 36188 | 55 | 45 | 42 | 47 | 7 |
| Capros @ 100 ug/ml + | 30426 | 34509 | 30327 | 51 | 45 | 52 | 49 | 4 |
| Temozolomide @ 300 uM | | | | | | | | |
| Capros @ 100 ug/ml + | 22936 | 30624 | 30757 | 63 | 51 | 51 | 55 | 7 |
| Temozolomide @ 100 uM | | | | | | | | |
| Capros @ 100 ug/ml + | 28445 | 34230 | 36564 | 55 | 45 | 42 | 47 | 7 |
| Temozolomide @ 30 uM | | | | | | | | |
| Ayuric @ 100 ug/ml + | 22662 | 27655 | 26418 | 64 | 56 | 58 | 59 | 4 |
| Temozolomide @ 300 uM | | | | | | | | |
| Ayuric @ 100 ug/ml + | 21383 | 26609 | 26896 | 66 | 58 | 57 | 60 | 5 |
| Temozolomide @ 100 uM | | | | | | | | |
| Ayuric @ 100 ug/ml + | 21535 | 28083 | 28708 | 66 | 55 | 54 | 58 | 6 |
| Temozolomide @ 30 uM | | | | | | | | |
| Ayuflex @ 100 ug/ml + | 13705 | 16899 | 16306 | 78 | 73 | 74 | 75 | 3 |
| Capros @ 100 ug/ml | | | | | | | | |
| Ayuflex @ 100 ug/ml + | 8003 | 11131 | 10051 | 87 | 82 | 84 | 84 | 3 |
| Ayuric @ 100 ug/ml | | | | | | | | |
| Capros @ 100 ug/ml + | 15730 | 18388 | 16458 | 75 | 71 | 74 | 73 | 2 |
| Ayuric @ 100 ug/ml | | | | | | | | |
| Temozolomide @ 300 uM | 45178 | 54144 | 53028 | 28 | 14 | 15 | 19 | 8 |
| Temozolomide @ 100 uM | 36970 | 45539 | 44666 | 41 | 27 | 29 | 32 | 8 |
| Temozolomide @ 30 uM | 37016 | 52703 | 53038 | 41 | 16 | 15 | 24 | 15 |
| Doxorubicin @ 10 uM | 1418 | 1584 | 2381 | 98 | 97 | 96 | 97 | 1 |

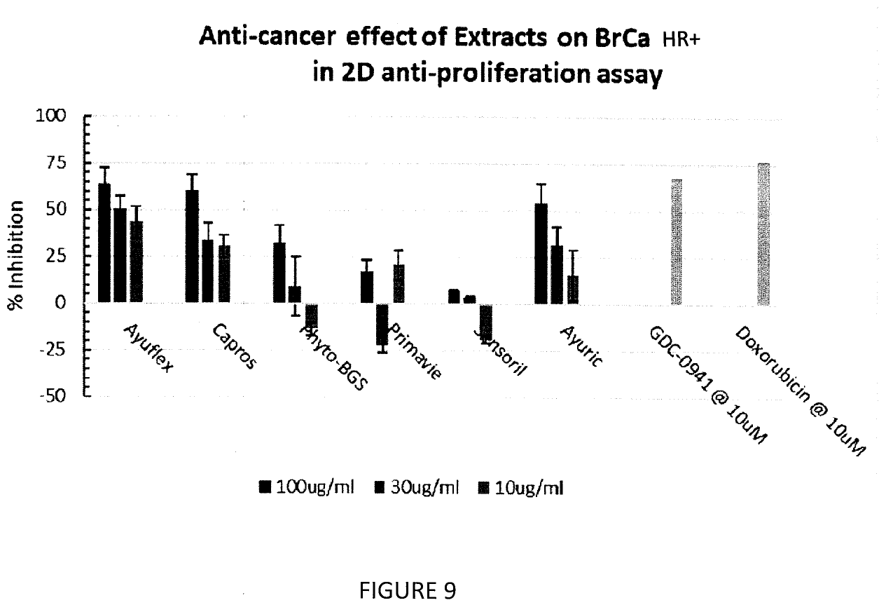

FIG. 9 represents information provided in Tables 17-20B, showing the inhibition of proliferation of breast cancer cells (“HR+”, ER/PR+ Her2 Equivocal) in the anti-proliferation assay. FIG. 9 compares the inhibition of proliferation of Breast cancer “HR+” PDCs by the 6 different extracts in the assay, showing the highest inhibition by the application of control drugs GDC-0941 (pictilisib, 10 uM) and doxorubicin (10 uM). AyuFlex® showed the most potent anti-proliferative effects on these cancer cells, comparable to GDC-0941 (pictilisib), closely followed by Capros®, and then Ayuric®. Effects by PhytoBGS®, PrimaVie®, and Sensoril® are also depicted in FIG. 9. Bars shown from left to right for each extract represent the application of 100 ug extract/ml, 30 ug extract/ml, and 10 ug extract/ml to the cell wells.

TABLE 17
Assay Controls

| | | | Assay | |
|---|---|---|---|---|
| | Avg | SD | fold | Z′ |
| 100% Proliferation (Cell Control) | 64398 | 5624 | 1966 | 0.74 |
| 0% Proliferation (Media only) | 33 | 20 | | |

TABLE 18
Assay Drug Controls
GDC-0941 @ 10 uM

| Avg | | % Inhibition | | |
|---|---|---|---|---|
| n1 | n2 | n1 | n2 | Avg |
| 19603 | 21420 | 70 | 67 | 68 |

TABLE 19
Assay Drug Controls
Doxorubicin @ 10 uM

| Avg | | % Inhibition | | |
|---|---|---|---|---|
| n1 | n2 | n1 | n2 | Avg |
| 16240 | 13292 | 75 | 79 | 77 |

TABLE 20A
Assay Results

| | % Inhibition | | | | | | | |
|---|---|---|---|---|---|---|---|---|
| | 100 ug/ml | | 30 ug/ml | | | 10 ug/ml | | |
| Ayuflex ® | 70 | 57 | 58 | 48 | 45 | 53 | 37 | 39 |
| Capros ® | 67 | 54 | 44 | 30 | 26 | 35 | 33 | 23 |
| Phyto-BGS ® | 39 | 26 | 22 | −9 | 14 | 8* | −16 | −11 |
| Primavie ® | 13 | 21 | 6* | −25 | −19 | 26 | 15 | −20 |
| Sensoril ® | 8 | −12 | 3 | 4 | −17 | 11 | −21 | −18 |
| Ayuric ® | 61 | 46 | 42 | 29 | 24 | 31 | 12 | 5 |

TABLE 20B
Assay Results

| | 100 ug/ml | | 30 ug/ml | | 10 ug/ml | |
|---|---|---|---|---|---|---|
| | Avg % | | Avg % | | Avg % | |
| | inhibition | SD | inhibition | SD | inhibition | SD |
| Ayuflex ® | 64 | 9 | 50 | 7 | 43 | 8 |
| Capros ® | 60 | 9 | 34 | 9 | 30 | 6 |
| Phyto-BGS ® | 32 | 9 | 9 | 16 | −13 | 4 |
| Primavie ® | 17 | 6 | −22 | 4 | 21 | 8 |
| Sensoril ® | −2 | 14 | −4 | 12 | −9 | 18 |
| Ayuric ® | 54 | 11 | 32 | 9 | 16 | 13 |

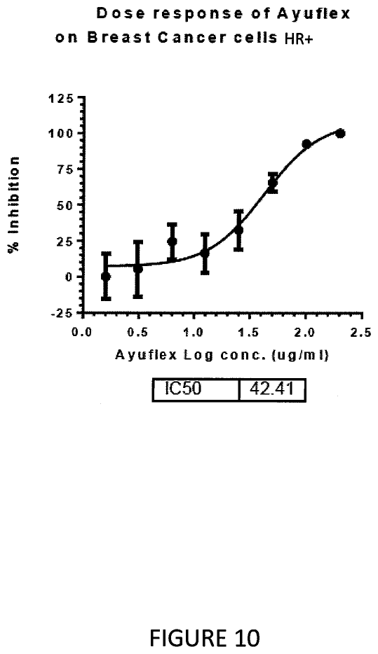

FIG. 10 shows a dose-response curve of Ayuflex® on HR+ breast cancer cells (that is, breast cancer cells that are ER/PR+ Her2 equivocal). As shown in FIG. 9 and Table 20B, AyuFlex® showed 50% inhibition of HR+ breast cancer cells at 30 ug/ml doses, tested at 3 graded doses. The S-shaped dose response curve in FIG. 10 reveals an IC₅₀ (Inhibitory Concentration 50) of about 42.41 ug Ayuflex®/ml. Table 21 shows data used to generate the dose-response curve.

TABLE 21
Dose-response curve of AyuFlex ® on HR+ breast cancer cells
Ayuflex DRC

| | | | Raw data | | % Inhibition | | |
|---|---|---|---|---|---|---|---|
| Doses | Conc. | | n1 | n2 | n1 | n2 | Avg |
| 1 | 200 | ug/ml | 81 | 80 | 100 | 100 | 100 |
| 2 | 100 | ug/ml | 6192 | 7135 | 93 | 92 | 93 |
| 3 | 50 | ug/ml | 27345 | 35402 | 70 | 61 | 65 |
| 4 | 25 | ug/ml | 51896 | 68951 | 42 | 23 | 33 |
| 5 | 12.5 | ug/ml | 66658 | 83762 | 26 | 7 | 16 |
| 6 | 6.3 | ug/ml | 59876 | 75427 | 33 | 16 | 25 |
| 7 | 3.1 | ug/ml | 72667 | 96931 | 19 | −8 | 5 |
| 8 | 1.6 | ug/ml | 79455 | 99926 | 11 | −11 | 0 |

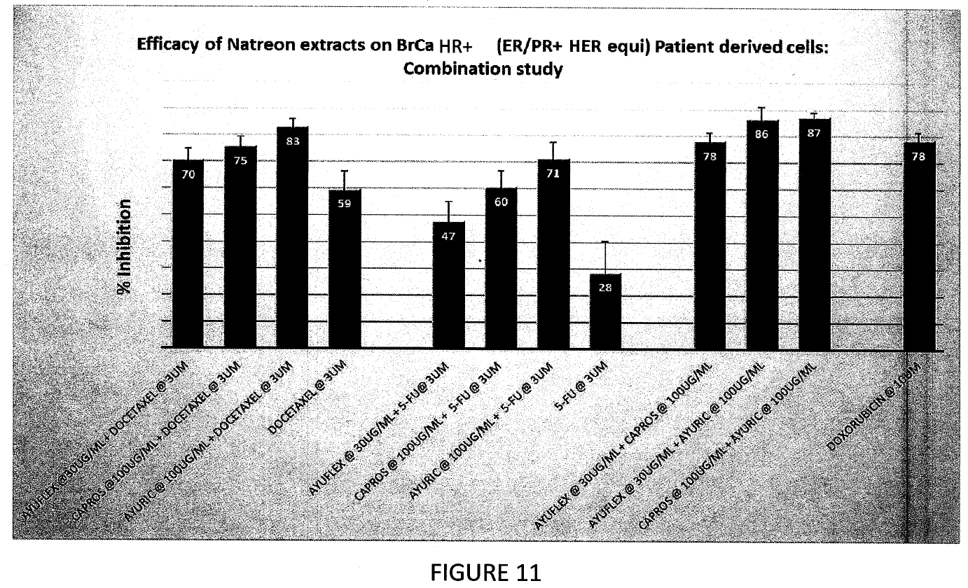

FIG. 11 represents information provided in Table 22, showing a significant anti-cancer inhibition of HR+(ER/PR+ Her2equivocal) proliferation in combination with docetaxel (an antimicrotubule agent that inhibits spindle assembly during mitosis) or 5-fluorouracil (5-FU).

TABLE 22
Inhibition of HR+ with AyuFlex ®, Capros ®, Ayuric ®

| | Raw data | | | % Inhibition | | | | |
|---|---|---|---|---|---|---|---|---|
| Combination | n1 | n2 | n3 | n1 | n2 | n3 | Avg | SD |
| Ayuflex @ 30 ug/ml + | 24851 | 31307 | 24077 | 72 | 65 | 73 | 70 | 4 |
| Docetaxel @ 3 uM | | | | | | | | |
| Capros @ 100 ug/ml + | 18138 | 25149 | 23083 | 80 | 72 | 74 | 75 | 4 |
| Docetaxel @ 3 uM | | | | | | | | |
| Ayuric @ 100 ug/ml + | 12153 | 16841 | 17627 | 86 | 81 | 80 | 83 | 3 |
| Docetaxel @ 3 uM | | | | | | | | |
| Docetaxel @ 3 uM | 29734 | 41946 | 37833 | 67 | 53 | 58 | 59 | 7 |
| Ayuflex @ 30 ug/ml + | 39229 | 51560 | 50871 | 56 | 43 | 43 | 47 | 8 |
| 5-FU @ 3 uM | | | | | | | | |
| Capros @ 100 ug/ml + | 29313 | 39767 | 37433 | 67 | 56 | 58 | 60 | 6 |
| 5-FU @ 3 uM | | | | | | | | |
| Ayuric @ 100 ug/ml + | 19508 | 30418 | 27860 | 78 | 66 | 69 | 71 | 6 |
| 5-FU @ 3 uM | | | | | | | | |
| 5-FU @ 3 uM | 51975 | 71897 | 69012 | 42 | 20 | 23 | 28 | 12 |
| Ayuflex @ 30 ug/ml + | 17047 | 23164 | 19543 | 81 | 74 | 78 | 78 | 3 |
| Capros @ 100 ug/ml | | | | | | | | |
| Ayuflex @ 30 ug/ml + | 7683 | 14692 | 14489 | 91 | 84 | 84 | 86 | 4 |
| Ayuric @ 100 ug/ml | | | | | | | | |
| Capros @ 100 ug/ml + | 9840 | 13176 | 12303 | 89 | 85 | 86 | 87 | 2 |
| Ayuric @ 100 ug/ml | | | | | | | | |
| Doxorubicin @ 10 uM | 17135 | 18734 | 22648 | 81 | 79 | 75 | 78 | 3 |

FIG. 11 shows that all three extracts—Ayuflex®, Capros®, and Ayuric®—in combination with docetaxel, showed increased, synergistic inhibition of HR+ breast cancer cell proliferation than when tested alone. 3 uM docetaxel applied to HR+ breast cancer cells in the antiproliferation assay described above inhibited HR+ breast cancer cells by about 59%. As shown in FIG. 9 and Table 20B above, Ayuflex® alone (30 ug/ml), Capros® alone (100 ug/ml), and Ayuric® alone (100 ug/ml) inhibited HR+breast cancer cells by about 50%, 60%, and 54%, respectively. FIG. 11 shows that extracts Ayuflex® (30 ug/ml), Capros® (100 ug/ml), and Ayuric® (100 ug/ml), combined with 3 uM docetaxel, inhibited proliferation of HR+ breast cancer cells by about 70%, 75%, and 83% respectively.

FIG. 11 also shows that all three extracts—Ayuflex®, Capros®, and Ayuric®—in combination with 5-fluorouracil (5-FU), showed increased, synergistic inhibition of HR+ breast cancer cell proliferation than when tested alone. 3 uM 5-FU applied to HR+ breast cancer cells in the antiproliferation assay described above inhibited HR+ breast cancer cells by about 28%. As shown in FIG. 9 and Table 20B above, Ayuflex® alone (30 ug/ml), Capros® alone (100 ug/ml), and Ayuric® alone (100 ug/ml) inhibited HR+ breast cancer cells by about 50%, 60%, and 54%, respectively. FIG. 11 shows that extracts Ayuflex® (30 ug/ml), Capros® (100 ug/ml), and Ayuric® (100 ug/ml), combined with 3 uM 5-FU, inhibited proliferation of HR+ breast cancer cells by about 47%, 60%, and 71% respectively.

Also, FIG. 11 shows that Ayuflex® and Capros® combined; Ayuflex® and Ayuric® combined; and Capros® and Ayuric® combined substantially increased inhibition of HR+ breast cancer cell proliferation over Ayuflex® (50%), Capros® (60%), and Ayuric® (54%) alone. The combination of 30 ug/ml Ayuflex® and 100 ug/ml Capros® resulted in about 78% inhibition of HR+ breast cancer cell proliferation, while the combination of 30 ug/ml Ayuflex® and 100 ug/ml Ayuric®, and of 100 ug/ml Capros® and 100 ug/ml Ayuric® inhibited HR+ breast cancer cell inhibition by about 86% and 87%, respectively. Control drug doxorubicin (10 uM) is shown as inhibiting HR+ breast cancer cell proliferation by 78%, at the far right of FIG. 11.

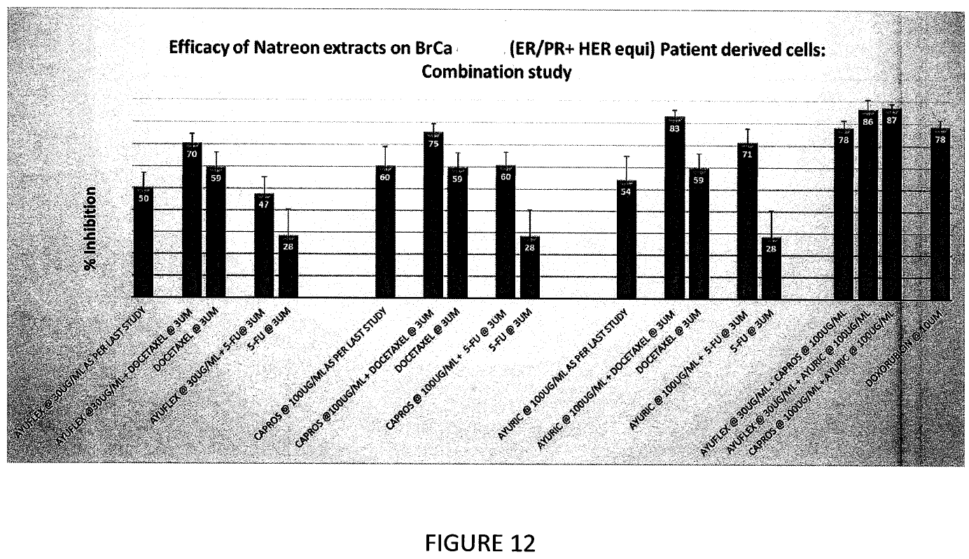

FIG. 12 compiles results shown in FIG. 11 and Table 22 according to the extract studied (that is, Ayuflex®, Capros®, Ayuric®). Ayuflex® (30 ug/ml) combined with 3 uM docetaxel inhibited proliferation of HR+ breast cancer cells by 70%, compared with 59% inhibition by docetaxel alone, 50% inhibition by Ayuflex® alone, and 47% inhibition when combined with 3 uM 5-FU. 3 uM 5-FU alone inhibited cell proliferation by 29%.

Capros® (100 ug/ml) combined with 3 uM docetaxel inhibited proliferation of HR+ breast cancer cells by 75%, compared with 59% inhibition by docetaxel alone, 60% inhibition by Capros® alone, and 60% inhibition when combined with 3 uM 5-FU. 3 uM 5-FU alone inhibited cell proliferation by 29%.

Ayuric® (100 ug/ml) combined with 3 uM docetaxel inhibited proliferation of HR+ breast cancer cells by 83%, compared with 59% inhibition by docetaxel alone, 54% inhibition by Ayuric® alone, and 71% inhibition when combined with 3 uM 5-FU. 3 uM 5-FU alone inhibited cell proliferation by 29%.

Each of the 3 extracts studied—Ayuflex®, Capros®, Ayuric®—showed better inhibition when used in combination with marketed drugs docetaxel and 5-FU than compared to the marketed drugs alone. Also, when these extracts were tested in the antiproliferative assay in combination with each other, for any combination of 2 of the extracts (Ayuflex® 30 ug/ml, Capros® 100 ug/ml, Ayuric® 100 ug/ml), a very good inhibition of HR+ breast cancer cell proliferation was observed, at greater than 78%. See FIG. 12, showing the combination of Ayuflex® and Capros® as inhibiting cell proliferation by 78%, Ayuflex® and Ayuric® at 86%, and Capros® and Ayuric® at 87%. For comparison, 10 uM doxorubicin is shown at the far right of FIG. 12 as inhibiting proliferation of these cells by 78%.

As shown in FIG. 9, when tested at 3 different doses, Ayuflex® showed the highest anti-cancer activity for HR+ breast cancer cells, followed by Capros® and then Ayuric®. Accordingly, the order of anti-cancer activity for the extracts alone is Ayuflex®>Capros®>Ayuric®. However, in FIGS. 11 and 12, when these extracts were tested on the same HR+ breast cancer PDCs in combination with docetaxel and 5-FU, surprisingly, Ayuric® showed the best additive activity, followed by Capros®, and then Ayuflex®. Accordingly, the order of anti-cancer activity for the combination of these extracts with docetaxel or 5-FU is Ayuric®+Drug X>Capros®+Drug X>Ayuflex®+Drug X.

Overall, all the three extracts—Ayuflex®, Capros®, and Ayuric®—showed strong anti-cancer activity against HR+ breast cancer PDCs. The cells are highly aggressive in culture; the finding and confirmation that each extract can substantially inhibit the PDCs proliferation in significant percentages in 72 hours, alone or in combination, was surprising and unexpected.

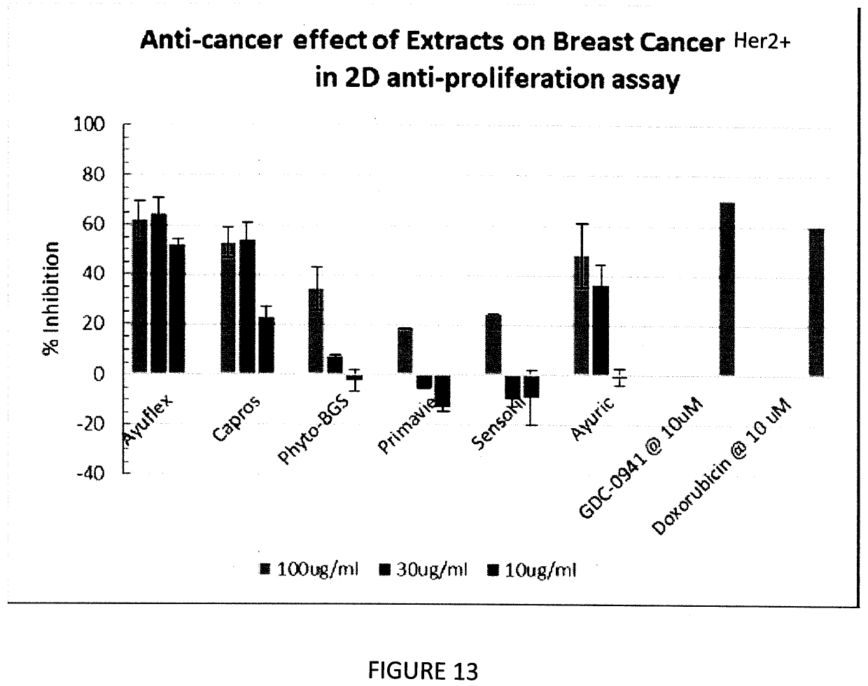

FIG. 13 represents information provided in Tables 23-26B, showing the inhibition of proliferation of breast cancer cells (“Her2+”, ER/PR− Her2+) in the anti-proliferation assay described above. FIG. 13 compares the inhibition of proliferation of Breast cancer “Her2+” PDCs by the 6 different extracts in the assay. The result of application of 10 uM GDC-0941 (pictilisib) and 10 uM doxorubicin as anti-cancer drug controls are shown to the right of the Figure. AyuFlex® showed the most potent anti-proliferative effects of the 6 extracts tested on these cancer cells, comparable to doxorubicin, closely followed by Capros®, and then Ayuric®. Effects by PhytoBGS®, PrimaVie®, and Sensoril® are also depicted in FIG. 13.

TABLE 23
Assay Controls

| | | | Assay | |
|---|---|---|---|---|
| | Avg | SD | fold | Z′ |
| 100% Proliferation (Cell Control) | 75052 | 7174 | 1464 | 0.71 |
| 0% Proliferation (Media only) | 51 | 22 | | |

TABLE 24
Assay Drug Control
GDC-0941 @ 10 uM

| Avg | | % Inhibition | | |
|---|---|---|---|---|
| n1 | n2 | n1 | n2 | Avg |
| 21823 | 23075 | 71 | 69 | 70 |

TABLE 25
Assay Drug Control
Doxorubicin @ 10 uM

| Avg | | % Inhibition | | |
|---|---|---|---|---|
| n1 | n2 | n1 | n2 | Avg |
| 28714 | 31853 | 62 | 58 | 60 |

TABLE 26A
Assay Results

| | % Inhibition | | | | | | | |
|---|---|---|---|---|---|---|---|---|
| | 100 ug/ml | | 30 ug/ml | | | 10 ug/ml | | |
| Ayuflex ® | 67 | 56 | 72 | 60 | 60 | 53 | 49 | 54 |
| Capros ® | 57 | 48 | 62 | 51 | 49 | 26 | 18 | 24 |
| Phyto-BGS ® | 29 | 40 | 35* | 8 | 7 | 3 | −5 | −4 |
| Primavie ® | 19 | −1 | 11* | −5 | −5 | 13* | −12 | −14 |
| Sensoril ® | 24 | −6 | 11* | −12 | −7 | 3 | −14 | −17 |
| Ayuric ® | 57 | 39 | 44 | 28 | 37 | 13* | −3 | 2 |

TABLE 26B
Assay Results

| | 100 ug/ml | | 30 ug/ml | | 10 ug/ml | |
|---|---|---|---|---|---|---|
| | Avg % | | Avg % | | Avg % | |
| | inhibition | SD | inhibition | SD | inhibition | SD |
| Ayuflex ® | 62 | 8 | 64 | 7 | 52 | 3 |
| Capros ® | 53 | 6 | 54 | 7 | 23 | 5 |
| Phyto-BGS ® | 34 | 8 | 7 | 1 | −2 | 4 |
| Primavie ® | 19 | 0 | −5 | 0 | −13 | 2 |
| Sensoril ® | 24 | 0 | −9 | 3 | −9 | 11 |
| Ayuric ® | 48 | 13 | 36 | 8 | −1 | 3 |

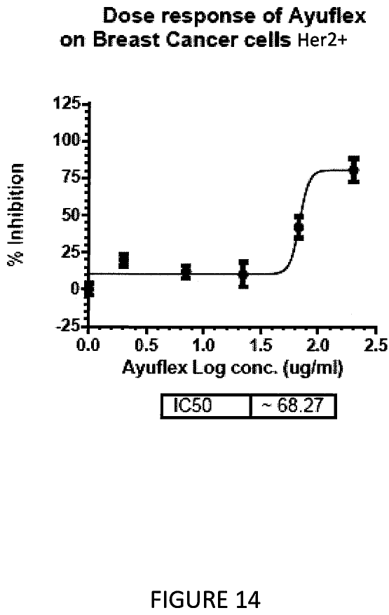

As Ayuflex® showed the highest anti-cancer, anti-proliferative activity of the extracts tested in FIG. 13, testing with the aim of revealing a dose-response curve and the IC₅₀ of Ayuflex® for Her2+ breast cancer cells was performed. FIG. 14 shows a dose-response curve of Ayuflex® on Her2+ breast cancer cells. As shown in FIG. 13, AyuFlex® showed 50% inhibition of TN breast cancer cells at a 10 ug/ml dose, and was tested at 3 graded doses. The dose response curve in FIG. 14 reveals an IC₅₀ of about 68.27 ug Ayuflex®/ml. Table 27 shows data used to generate the dose-response curve.

TABLE 27
Ayuflex Dose-Response Curve
Ayuflex DRC

| | | |
|---|---|---|
| Conc. | Raw data | % Inhibition |

| Doses | (ug/ml) | n1 | n2 | n1 | n2 | Avg |
|---|---|---|---|---|---|---|
| 1 | 200 | 4617 | 8293 | 86 | 75 | 81 |
| 2 | 67 | 17613 | 20848 | 47 | 37 | 42 |
| 3 | 22 | 27891 | 32137 | 16 | 4 | 10 |
| 4 | 7 | 28432 | 30269 | 15 | 9 | 12 |
| 5 | 2 | 25593 | 27659 | 23 | 17 | 20 |
| 6 | 1 | 34443 | 32209 | −3 | 3 | 0 |
| 7 | 0 | 30800 | 33752 | 8 | −1 | 3 |
| 8 | 0 | 27215 | 30136 | 18 | 10 | 14 |

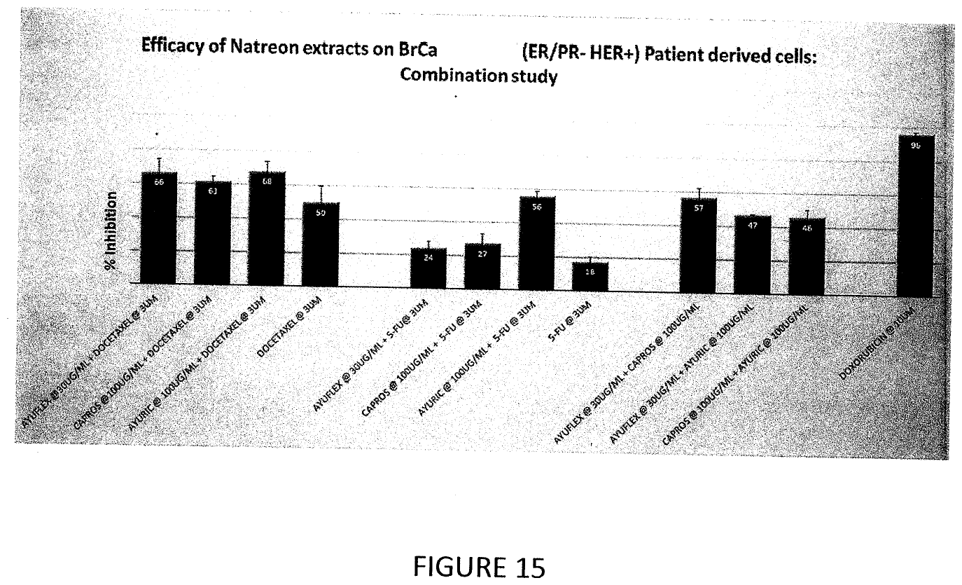

FIG. 15 represents information provided in Table 28, showing a significant anti-cancer inhibition of Her2+ breast cancer cell proliferation by extracts in combination with 3 uM docetaxel or 3 uM 5-fluorouracil (5-FU). Ayuflex® and docetaxel together inhibited Her2+ cell proliferation by 66% and Ayuric® and docetaxel by 68%, with Capros® and docetaxel inhibiting Her2+ by about 61%. As shown in FIG. 15, docetaxel alone inhibited proliferation of the Her2+ cells by about 50%.

In contrast, 5-FU in combination with Ayuflex® (30 ug/ml) and Capros® (100 ug/ml), inhibited proliferation by about 24% and 27% respectively, as compared with 18% by 5-FU alone. FIG. 13 shows that Ayuric® inhibited proliferation of Her2+ cells by about 34%, however, Ayuric® and 5-FU together appeared to work synergistically, inhibiting proliferation by about 56%.

Finally, combinations of the three extracts share similarities in their inhibition of Her2+ cells (57%, 47%, 46% respectively for Ayuflex®/Capros®, Ayuflex®/Ayuric®, and Capros®/Ayuric®). With the exception of the combination of Ayuric® and 5-FU, the extracts share similarities in their inhibition of Her2+ cells when used in combination with docetaxel and 5-FU. It is noted that the PDCs used in this experiment were slow-growing and differed in morphology. Also, a new stock of Ayuflex® was prepared for this study, which showed a little less potency than stock used in some previous experiments.

TABLE 28
Inhibition of Her2+ breast cancer cell proliferation

| | Raw data | | | % inhibition | | | | |
|---|---|---|---|---|---|---|---|---|
| Combination | n1 | n2 | n3 | n1 | n2 | n3 | Avg | SD |
| Ayuflex @30 ug/ml + | 8318 | 13508 | 11954 | 75 | 60 | 64 | 66 | 8 |
| Docetaxel @ 3 uM | | | | | | | | |
| Capros @100 ug/ml + | 11793 | 13674 | 13069 | 65 | 59 | 61 | 61 | 3 |
| Docetaxel @ 3 uM | | | | | | | | |
| Ayuric @ 100 ug/ml + | 9029 | 12954 | 10392 | 73 | 61 | 69 | 68 | 6 |
| Docetaxel @ 3 uM | | | | | | | | |
| Docetaxel @ 3 uM | 23284 | 18884 | 14463 | 30* | 43 | 57 | 50 | 9 |
| Ayuflex @ 30 ug/ml + | 24373 | 26114 | 34166 | 27 | 22 | −3 | 24 | 4 |
| 5-FU@ 3 uM | | | | | | | | |
| Capros @ 100 ug/ml + | 22889 | 25557 | 17709 | 31 | 23 | 47* | 27 | 6 |
| 5-FU @ 3 uM | | | | | | | | |
| Ayuric @ 100 ug/ml + | 15454 | 20125 | 14086 | 54 | 40* | 58 | 56 | 3 |
| 5-FU @ 3 uM | | | | | | | | |
| 5-FU @ 3 uM | 19643 | 28153 | 26851 | 41* | 16 | 19 | 18 | 3 |
| Ayuflex @ 30 ug/ml + | 13209 | 19318 | 15708 | 60 | 42* | 53 | 56 | 5 |
| Capros @ 100 ug/ml | | | | | | | | |
| Ayuflex @ 30 ug/ml + | 12903 | 17628 | 17705 | 61* | 47 | 47 | 47 | 0 |
| Ayuric @ 100 ug/ml | | | | | | | | |
| Capros @ 100 ug/ml + | 11840 | 17003 | 18991 | 65* | 49 | 43 | 46 | 4 |
| Ayuric @ 100 ug/ml | | | | | | | | |
| Doxorubicin @ 10 uM | 1530 | 1667 | 996 | 95 | 95 | 97 | 96 | 1 |

*Outliers

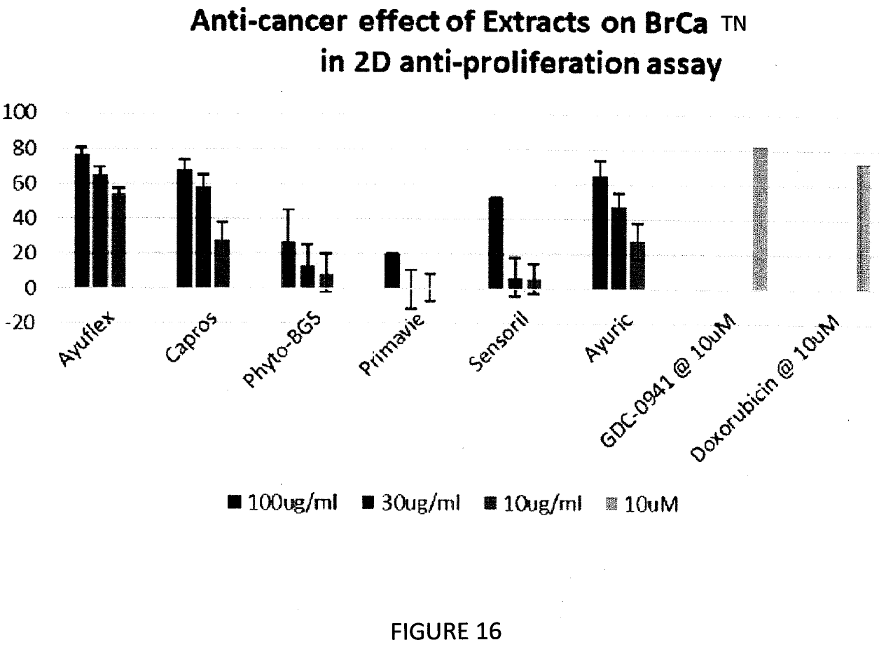

FIG. 16 represents information provided in Tables 29-32B, showing the inhibition of proliferation of breast cancer cells (“TN”, Triple Negative) in the anti-proliferation assay. FIG. 16 compares the inhibition of proliferation of Breast cancer “Triple Negative” PDCs by the 6 different extracts in the assay, with GDC-0941 (pictilisib) and doxorubicin shown at the right side of FIG. 16 as drug controls known to inhibit the proliferation of cancer cells. AyuFlex® showed the most potent anti-proliferative effects on these cancer cells, comparable to GDC-0941 and doxorubicin, closely followed by Capros®, Ayuric®, and then Sensoril®, showing anti-proliferative activity in the 100 ug/ml concentration applied to cells. Effects by PhytoBGS® and PrimaVie® are also depicted in FIG. 16.

TABLE 29
Assay Controls

| | | | Assay | |
|---|---|---|---|---|
| | Avg | SD | fold | Z′ |
| 100% Proliferation (Cell Control) | 71278 | 6637 | 1262 | 0.72 |
| 0% Proliferation (Media only) | 57 | 18 | | |

TABLE 30
Assay Drug Control
GDC-0941 @ 10 uM

| Avg | | % Inhibition | | |
|---|---|---|---|---|
| n1 | n2 | n1 | n2 | Avg |
| 11517 | 12897 | 84 | 82 | 83 |

TABLE 31
Assay Drug Control
Doxorubicin @ 10 uM

| Avg | | % Inhibition | | |
|---|---|---|---|---|
| n1 | n2 | n1 | n2 | Avg |
| 19364 | 19643 | 73 | 72 | 73 |

TABLE 32A
Assay Results

| | % Inhibition | | | | | | | |
|---|---|---|---|---|---|---|---|---|
| | 100 ug/ml | | 30 ug/ml | | | 10 ug/ml | | |
| Ayuflex ® | 79 | 74 | 70 | 63 | 63 | 58 | 52 | 53 |
| Capros ® | 72 | 64 | 66 | 55 | 52 | 39 | 22 | 24 |
| Phyto-BGS ® | 40 | 15 | 26 | 2 | 10 | 22 | 3 | 3 |
| Primavie ® | 84 | 20 | 9 | −12 | 2 | 10 | −4 | −3 |
| Sensoril ® | 52 | 19 | 19 | −3 | 4 | 16 | 0 | 2 |
| Ayuric ® | 71 | 58 | 56 | 41 | 45 | 38 | 18 | 27 |

TABLE 32B
Assay Results

| | 100 ug/ml | | 30 ug/ml | | 10 ug/ml | |
|---|---|---|---|---|---|---|
| | Avg % | | Avg % | | Avg % | |
| | inhibition | SD | inhibition | SD | inhibition | SD |
| Ayuflex ® | 77 | 4 | 65 | 4 | 54 | 3 |
| Capros ® | 68 | 6 | 58 | 8 | 28 | 10 |
| Phyto-BGS ® | 27 | 18 | 13 | 12 | 9 | 11 |
| Primavie ® | 20 | 0 | 0 | 11 | 1 | 8 |
| Sensoril ® | 35 | 24 | 7 | 11 | 6 | 8 |
| Ayuric ® | 65 | 9 | 47 | 8 | 28 | 10 |

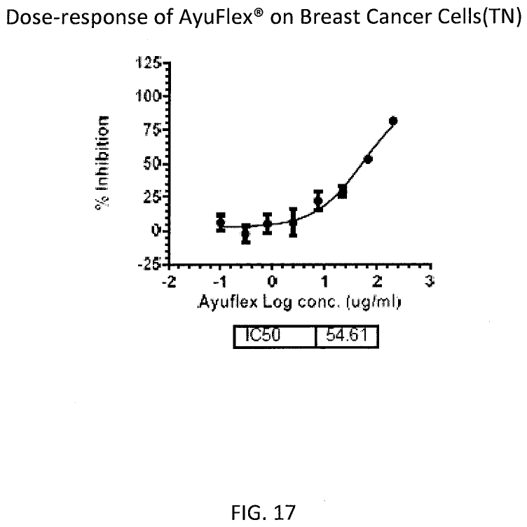

As Ayuflex® showed the highest anti-cancer, anti-proliferative activity of the extracts tested in FIG. 16, testing with the aim of revealing a dose-response curve and the IC₅₀ of Ayuflex® for TN breast cancer cells was performed. FIG. 17 shows a dose-response curve of Ayuflex® on TN (Triple Negative) breast cancer cells. AyuFlex® showed 50% inhibition of TN breast cancer cells at a 10 ug/ml dose, when tested at 3 graded doses. The dose response curve in FIG. 17 reveals an IC₅₀ of about 54.61 ug Ayuflex®/ml. Table 33 shows data used to generate the dose-response curve.

TABLE 33
Ayuflex ® Dose-Response Curve
Ayuflex DRC

| | | |
|---|---|---|
| Conc. | Raw data | % Inhibition |

| Doses | (ug/ml) | n1 | n2 | n1 | n2 | Avg |
|---|---|---|---|---|---|---|
| 1 | 200 | 12870 | 15012 | 83 | 80 | 82 |
| 2 | 66.7\| | 34601 | 36098 | 54 | 52 | 53 |
| 3 | 22.2 | 51275 | 55608 | 32 | 26 | 29 |
| 4 | 7.4 | 55229 | 62697 | 27 | 17 | 22 |
| 5 | 2.5 | 65914 | 76238 | 13 | −1 | 6 |
| 6 | 0.8 | 67950 | 75290 | 10 | 0 | 5 |
| 7 | 0.3 | 74107 | 80541 | 2 | −7 | −2 |
| 8 | 0.1 | 67946 | 74190 | 10 | 2 | 6 |

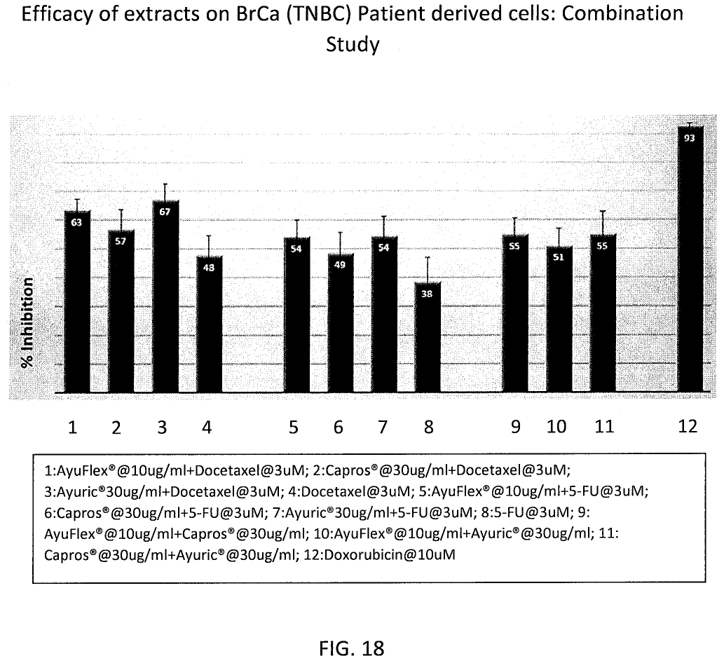

FIG. 18 represents information provided in Table 34, showing a significant anti-cancer inhibition of TN breast cancer cell proliferation in combination with 3 uM docetaxel or 3 uM 5-fluorouracil (5-FU). Ayuflex® (10 ug/ml) and docetaxel together inhibited TN cell proliferation by 63% and Ayuric® (30 ug/ml) and docetaxel by 67%, with Capros® (30 ug/ml) and docetaxel inhibiting TN cell proliferation by about 57%. Docetaxel alone inhibited proliferation of the TN cells by about 48%. In a previous study, Ayuflex® (10 ug/ml) inhibited TN cell proliferation by 54%; Capros® inhibited TN cell proliferation by 58%; and Ayuric® inhibited TN cell proliferation by 47%. The inhibition of proliferation by docetaxel and Ayuric® in particular shows synergistic results, as alone, each substance inhibited TN cell proliferation by 47-48%, but taken together, inhibition of TN cell proliferation increased nearly 20% to 67%.

FIG. 18 also shows that 5-FU (3 uM) in combination with Ayuflex® (10 ug/ml) or with Ayuric® (30 ug/ml), inhibited TN cell proliferation by 54%; and in combination with Capros®, inhibited TN cell proliferation by 49%, as compared with 38% by 5-FU alone. In addition, FIG. 18 shows that combinations of the three extracts inhibited TN cell proliferation by 55%, 51%, 55% respectively for Ayuflex® (10 ug/ml)/Capros® (30 ug/ml), Ayuflex® (10 ug/ml)/Ayuric® (30 ug/ml), and Capros® (30 ug/ml)/Ayuric® (30 ug/ml). Overall, the 3 extracts share similarities in their inhibition of TN cells, when tested alone, in combination with docetaxel and 5-FU, or in combination with each other.

TABLE 34
Inhibition of TN breast cancer cell proliferation

| | Raw data | | | % Inhibition | | | | |
|---|---|---|---|---|---|---|---|---|
| Combination | n1 | n2 | n3 | n1 | n2 | n3 | Avg | SD |
| Ayuflex @ 10 ug/ml + | 24205 | 29046 | 29650 | 68 | 62 | 61 | 63 | 4 |
| Docetaxel @ 3 uM | | | | | | | | |
| Capros @ 30 ug/ml + | 26716 | 35847 | 35282 | 65 | 53 | 53 | 57 | 7 |
| Docetaxel @ 3 uM | | | | | | | | |
| Ayuric @ 30 ug/ml + | 19960 | 26958 | 27891 | 74 | 64 | 63 | 67 | 6 |
| Docetaxel @ 3 uM | | | | | | | | |
| Docetaxel @ 3 uM | 33410 | 42476 | 42703 | 56 | 44 | 43 | 48 | 7 |
| Ayuflex @ 10 ug/ml + | 29616 | 38064 | 36091 | 61 | 50 | 52 | 54 | 6 |
| 5-FU @ 3 uM | | | | | | | | |
| Capros @ 30 ug/ml + | 32709 | 43186 | 40709 | 57 | 43 | 46 | 49 | 7 |
| 5-FU @ 3 uM | | | | | | | | |
| Ayuric @ 30 ug/ml + | 28675 | 36109 | 38462 | 62 | 52 | 49 | 54 | 7 |
| 5-FU @ 3 uM | | | | | | | | |
| 5-FU @ 3 uM | 39077 | 50221 | 50264 | 48 | 33 | 33 | 38 | 9 |
| Ayuflex @ 10 ug/ml + | 29096 | 36274 | 36747 | 61 | 52 | 51 | 55 | 6 |
| Capros @ 30 ug/ml | | | | | | | | |
| Ayuflex @ 10 ug/ml + | 32247 | 41305 | 37502 | 57 | 45 | 50 | 51 | 6 |
| Ayuric @ 30 ug/ml | | | | | | | | |
| Capros @ 30 ug/ml + | 27434 | 38949 | 35603 | 64 | 48 | 53 | 55 | 8 |
| Ayuric @ 30 ug/ml | | | | | | | | |
| Doxorubicin @ 10 uM | 4420 | 6305 | 5874 | 91 | 92 | 92 | 93 | 1 |

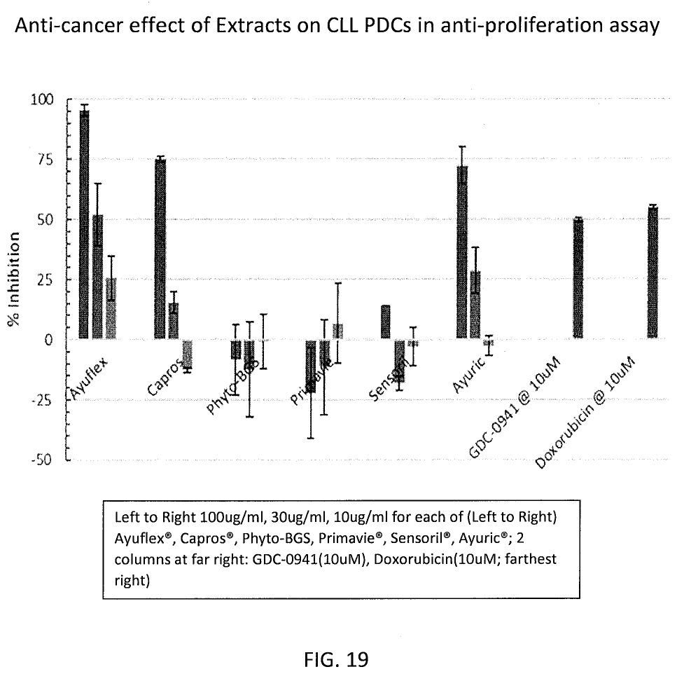

FIG. 19 represents information provided in Tables 35-38B, showing the inhibition of proliferation of chronic lymphocytic leukemia (CLL) PDCs in the anti-proliferation assay. FIG. 19 compares the inhibition of proliferation of CLL PDCs by the 6 different extracts in the anti-proliferation assay, showing nearly 100% inhibition of AML cell proliferation with the 100 ug/ml AyuFlex® extract concentration, and anti-proliferative activity of AyuFlex® comparable to drug standards GDC-0941 (pictilisib) and doxorubicin at the 30 ug/ml concentration. Capros® and Ayuric® outperformed the drug standards at their 100 ug/ml concentrations, showing about 75% anti-proliferation activity. Effects by PhytoBGS®, PrimaVie®, and Sensoril® are also depicted in FIG. 19.

TABLE 35
Assay Controls

| | | | Assay | |
|---|---|---|---|---|
| | Avg | SD | fold | Z′ |
| 100% Proliferation (Cell Control) | 5930 | 532 | 84 | 0.71 |
| 0% Proliferation (Media only) | 70 | 28 | | |

TABLE 36
Assay Drug Control
Doxorubicin @ 10 uM

| Avg | | % Inhibition | | |
|---|---|---|---|---|
| n1 | n2 | n1 | n2 | Avg |
| 3002 | 2367 | 50 | 61 | 55 |

TABLE 37
Assay Drug Control
GDC-0941 @ 10 uM

| Avg | | % Inhibition | | |
|---|---|---|---|---|
| n1 | n2 | n1 | n2 | Avg |
| 2562 | 3477 | 57 | 42 | 50 |

TABLE 38A
Assay Results

| | % Inhibition | | | | | | | |
|---|---|---|---|---|---|---|---|---|
| | 100 ug/ml | | 30 ug/ml | | | 10 ug/ml | | |
| Ayuflex ® | 97 | 94 | 66 | 41 | 49 | 36 | 24 | 18 |
| Capros ® | 76 | 74 | 38* | 12 | 19 | 19* | −13 | −12 |
| Phyto-BGS ® | 2 | −19 | 10 | −20 | −27 | 12 | −6 | −8 |
| Primavie ® | −9 | −36 | 9 | −12 | −31 | 26 | −3 | −3 |
| Sensoril ® | 14 | −16 | 4 | −16 | −20 | 3 | −43 | −8 |
| Ayuric ® | 78 | 67 | 39 | 25 | 21 | 18* | −6 | 0 |

TABLE 38B
Assay Results

| | 100 ug/ml | | 30 ug/ml | | 10 ug/ml | |
|---|---|---|---|---|---|---|
| | Avg % | | Avg % | | Avg % | |
| | inhibition | SD | inhibition | SD | inhibition | SD |
| Ayuflex | 95 | 2 | 52 | 13 | 26 | 9 |
| Capros | 75 | 1 | 15 | 4 | −13 | 1 |
| Phyto-BGS | −8 | 15 | −12 | 20 | −1 | 11 |
| Primavie | −22 | 19 | −12 | 20 | 7 | 17 |
| Sensoril | −1 | 21 | −11 | 13 | −16 | 24 |
| Ayuric | 73 | 8 | 29 | 10 | −3 | 4 |

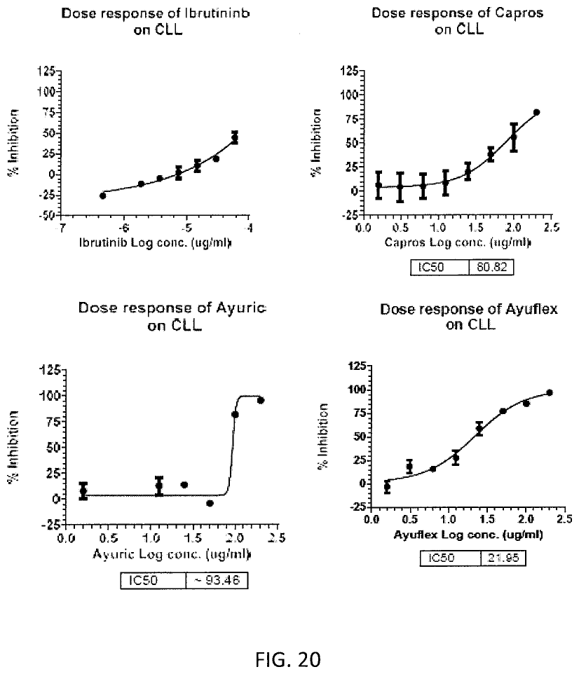

As previous testing showed Ayuflex®, Capros®, and Ayuric® shared good activity for inhibiting CLL PDC proliferation in the anti-proliferative assay discussed above, testing with the aim of revealing a dose-response curve and the IC₅₀ of Ayuflex®, Capros®, and Ayuric® for CLL PDCs was performed. FIG. 20 shows a dose-response curve of Ayuflex® on CLL cells, and an IC₅₀ of 21.95 ug/ml. The 22 ug/ml IC₅₀ for Ayuflex® was in line with about 50% inhibition observed with Ayuflex® previously. Also, an IC₅₀ of 80.82 ug/ml was identified for Capros® and for Ayuric®, an IC₅₀ of about 93.46 ug/ml. Tables 40-42 show data used to generate the dose-response curves for the extracts. A dose-response curve of the inhibition of CLL cells by ibrutinib is also shown in FIG. 20, and data to generate the curve shown in Table 39.

TABLE 39
Dose-response curve
Ibrutinib DRC

| | | Raw data | | % Inhibition | | |
|---|---|---|---|---|---|---|
| Doses | Conc. | n1 | n2 | n1 | n2 | Avg |
| 1 | 6.00E−05 | 20364 | 23884 | 49 | 40 | 44 |
| 2 | 3.00E−05 | 31715 | 32913 | 20 | 17 | 18 |
| 3 | 1.50E−05 | 33454 | 37044 | 15 | 6 | 11 |
| 4 | 7.50E−06 | 36853 | 40636 | 7 | −3 | 2 |
| 5 | 3.75E−06 | 41353 | 41370 | −5 | −5 | −5 |
| 6 | 1.88E−06 | 43486 | 44692 | −10 | −13 | −12 |
| 7 | 9.38E−07 | 157228 | 77748 | −299* | −97* | |
| 8 | 4.69E−07 | 78043 | 49566 | −98* | −26 | −26 |

*Outliers

TABLE 40
Dose-response curve
Ayuflex DRC

| | | | Raw data | | % Inhibition | | |
|---|---|---|---|---|---|---|---|
| Doses | Conc. | | n1 | n2 | n1 | n2 | Avg |
| 1 | 200 | ug/ml | 1274 | 1720 | 97 | 96 | 96 |
| 2 | 100 | ug/ml | 5815 | 132833 | 85 | −237* | 85 |
| 3 | 50 | ug/ml | 9035 | 45631 | 77 | −16* | 77 |
| 4 | 25 | ug/ml | 14360 | 18110 | 64 | 54 | 59 |
| 5 | 12.5 | ug/m | 26570 | 30474 | 33 | 23 | 28 |
| 6 | 6.3 | ug/ml | 32559 | 34113 | 18 | 14 | 16 |
| 7 | 3.1 | ug/ml | 30136 | 33881 | 24 | 14 | 19 |
| 8 | 1.6 | ug/ml | 38674 | 42196 | 2 | −7 | −2 |

*Outliers

TABLE 41
Dose-response curve
Ayuric DRC

| | | | Raw data | | % Inhibition | | |
|---|---|---|---|---|---|---|---|
| Doses | Conc. | | n1 | n2 | n1 | n2 | Avg |
| 1 | 200 | ug/ml | 1510 | 1974 | 96 | 95 | 96 |
| 2 | 100 | ug/ml | 6546 | 8141 | 84 | 80 | 82 |
| 3 | 50 | ug/ml | 118831 | 41081 | −202* | −4 | −4 |
| 4 | 25 | ug/ml | 53443 | 33759 | −36* | 14 | 14 |
| 5 | 12.5 | ug/ml | 32062 | 36771 | 19 | 7 | 13 |
| 6 | 6.3 | ug/ml | 174800 | 69322 | −344* | −76* | |
| 7 | 3.1 | ug/ml | 80265 | 49847 | −104* | −26 | −26 |
| 8 | 1.6 | ug/ml | 34358 | 38432 | 13 | 3 | 8 |

*Outliers

TABLE 42
Capros ® Dose-Response Curve
Capros ® DRC

| | | | Raw data | | % Inhibition | | |
|---|---|---|---|---|---|---|---|
| Doses | Conc. | | n1 | n2 | n1 | n2 | Avg |
| 1 | 200 | ug/ml | 6929 | 7493 | 83 | 81 | 82 |
| 2 | 100 | ug/ml | 13583 | 21326 | 66 | 46 | 56 |
| 3 | 50 | ug/ml | 22529 | 26420 | 43 | 33 | 38 |
| 4 | 25 | ug/ml | 29369 | 33909 | 26 | 14 | 20 |
| 5 | 12.5 | ug/ml | 32722 | 39366 | 17 | 0 | 9 |
| 6 | 6.3 | ug/ml | 34111 | 41002 | 14 | −4 | 5 |
| 7 | 3.1 | ug/ml | 33371 | 41891 | 15 | −6 | 5 |
| 8 | 1.6 | ug/ml | 33010 | 40669 | 16 | −3 | 7 |

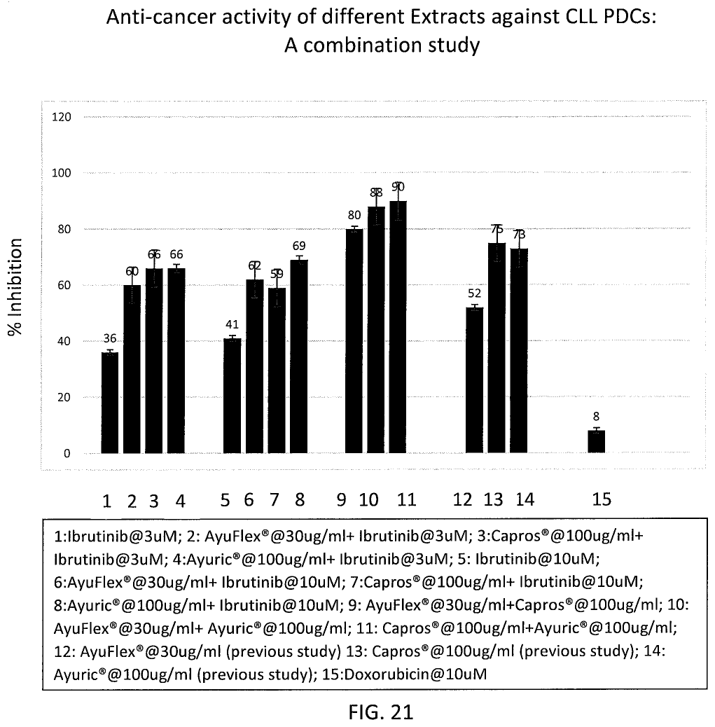

FIG. 21 shows, overall, that extracts of the present invention (Ayuflex®, Capros®, and Ayuric®) in combinations with each other showed better anti-cancer effects than extracts alone. Also, Ibrutinib showed a better inhibition profile for CLL cancer cells when tested in combination with these extracts than ibrutinib alone. Ibrutinib's effect can be enhanced with combined doses of extracts into subjects, according to the present invention. At the left side of FIG. 21, 3 uM ibrutinib alone inhibited CLL PDC proliferation by about 36%, however, in combination with Ayuflex® (30 ug/ml), Capros® (100 ug/ml) or Ayuric® (100 ug/ml), inhibition of proliferation increased to about 60% (Ayuflex®) or 66% (Capros®, Ayuric®). The inhibition of CLL cell proliferation by Ayuflex®, Capros®, or Ayuric® was previously measured at about 50% (Ayuflex®) and about 73-75% (Capros®, Ayuric®) (See right side of FIG. 21.)

Increasing the ibrutinib concentration to 10 uM increased the inhibition of CLL cell proliferation by ibrutinib alone by about 11%, to 41% inhibition of proliferation. In combination with Ayuflex® (30 ug/ml), Capros® (100 ug/ml) or Ayuric® (100 ug/ml), inhibition of proliferation of CLL cells changed to about 62% (Ayuflex®), 59% (Capros®), and 69% (Ayuric®). The highest level of inhibitory activity toward CLL cells shown in FIG. 21 is shown by combinations of extracts of the present invention (Ayuflex® (30 ug/ml), Capros® (100 ug/ml) or Ayuric® (100 ug/ml)). The Ayuflex®/Capros® combination shown in FIG. 21 inhibited proliferation of CLL cancer cells by 80%, while the Ayuflex®/Ayuric® combination and the Capros®/Ayuric® combination inhibited proliferation of the CLL PDCs by 88% and 90%, respectively. In contrast, as shown at the far right of FIG. 21, Ayuflex® (30 ug/ml) alone in a previous study inhibited CLL cell proliferation by 52%; Capros® (100 ug/ml), by 75%; and Ayuric® (100 ug/ml), by 73%. 10 uM doxorubicin showed relatively low inhibitory activity for CLL cells, as shown at the far right of FIG. 21. Table 43 shows data represented by FIG. 21.

TABLE 43
Inhibition of CLL PDC proliferation

| | | | | % Inhibition | | | | | | |
|---|---|---|---|---|---|---|---|---|---|---|
| | | | | Avg | SD | n1 | n2 | n3 | Avg | SD |
| Ibrutinib @ 3 uM | 20689 | 27455 | 28014 | 25386 | 4077 | 48 | 31 | 29 | 36 | 10 |
| Ayuflex @ 30 ug/ml + | 13369 | 18253 | 15428 | 15683 | 2452 | 66 | 54 | 61 | 60 | 6 |
| Ibrutinib @ 3 uM | | | | | | | | | | |
| Capros @ 100 ug/ml + | 11439 | 14698 | 14572 | 13570 | 1846 | 71 | 63 | 63 | 66 | 5 |
| Ibrutinib @ 3 uM | | | | | | | | | | |
| Ayuric @ 100 ug/ml + | 11783 | 12997 | 15371 | 13384 | 1825 | 70 | 67 | 61 | 66 | 5 |
| Ibrutinib @ 3 uM | | | | | | | | | | |
| Ibrutinib @ 10 uM | 19340 | 27230 | 173394* | 23285 | 5579 | 51 | 31 | * | 41 | 14 |
| Ayuflex @ 30 ug/ml + | 12542 | 17995 | 14819 | 15119 | 2739 | 68 | 55 | 63 | 62 | 7 |
| Ibrutinib @ 10 uM | | | | | | | | | | |
| Capros @ 100 ug/ml + | 18454 | 16505 | 13407 | 16122 | 2545 | 53 | 58 | 66 | 59 | 6 |
| Ibrutinib @ 10 uM | | | | | | | | | | |
| Ayuric @ 100 ug/ml + | 14344 | 12561 | 9754 | 12220 | 2314 | 64 | 68 | 75 | 69 | 6 |
| Ibrutinib @ 10 uM | | | | | | | | | | |
| Ayuflex @ 30 ug/ml + | 6094 | 9211 | 8212 | 7839 | 1592 | 85 | 77 | 79 | 80 | 4 |
| Capros @ 100 ug/ml | | | | | | | | | | |
| Ayuflex @ 30 ug/ml + | 5095 | 5394 | 4257 | 4915 | 589 | 87 | 87 | 89 | 88 | 1 |
| Ayuric @ 100 ug/ml | | | | | | | | | | |
| Capros @ 100 ug/ml + | 4269 | 4344 | 4088 | 4234 | 132 | 89 | 89 | 90 | 90 | 0 |
| Ayuric @ 100 ug/ml | | | | | | | | | | |
| Ayuflex @ 30 ug/ml | | | | | | | | | 52 | 13 |
| {previous study} | | | | | | | | | | |
| Capros @ 100 ug/ml | | | | | | | | | 75 | 1 |
| {previous study} | | | | | | | | | | |
| Ayuric @ 100 ug/ml | | | | | | | | | 73 | 8 |
| {previous study} | | | | | | | | | | |
| Doxorubicin @ 10 uM | 33348 | 43129 | 39159 | 38545 | 4919 | 16 | −9 | 1 | 2 | 13 |

*Outliers

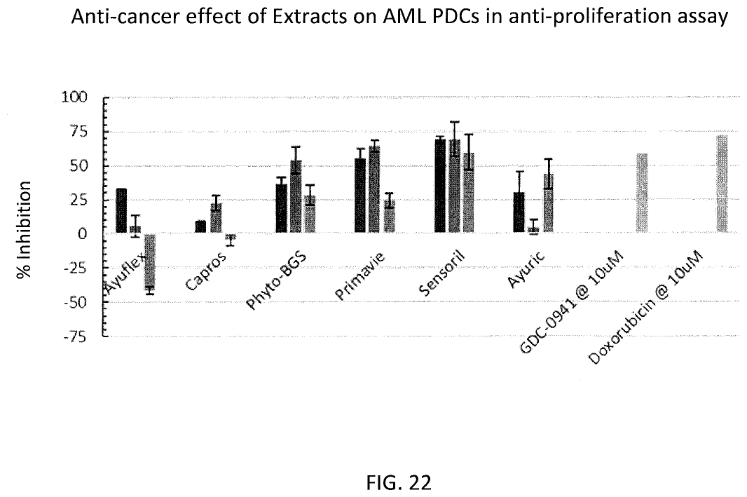

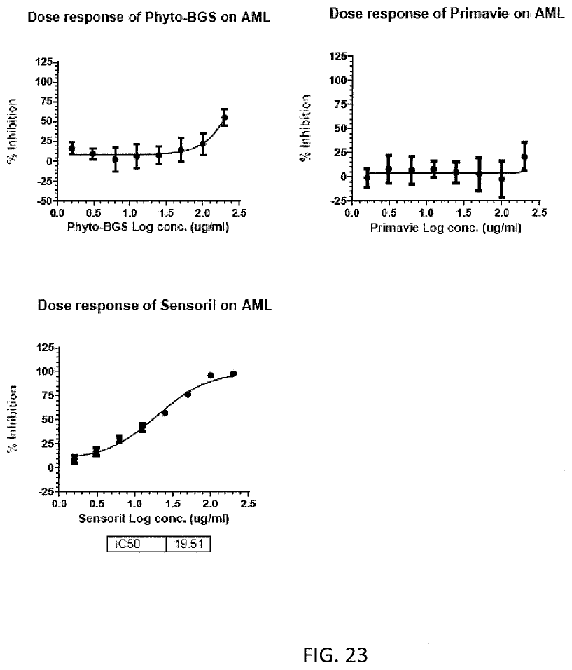

FIG. 22 represents information provided in Tables 44-47B, showing the inhibition of proliferation of acute myeloid leukemia (AML) PDCs in the anti-proliferation assay. FIG. 22 compares the inhibition of proliferation of AML PDCs by the 6 different extracts in the anti-proliferation assay, with Sensoril® matching or outperforming anti-proliferative activity by drug standards GDC-0941 (pictilisib) and doxorubicin at all concentrations tested (100 ug/ml, 30 ug/ml, 10 ug/ml). PrimaVie® showed comparable activity to Sensoril® in its 100 ug/ml and 30 ug/ml concentrations. The PhytoBGS® 30 ug/ml concentration and the Ayuric® 10 ug/ml concentration approached the anti-proliferation effects shown by drug standard GDC-0941. Effects by AyuFlex® and Capros® are also depicted in FIG. 22. GDC-0941 (pictilisib) and doxorubicin are presented as standard drug controls at the far right of FIG. 23.

TABLE 44
Assay Controls

| | | | Assay | |
|---|---|---|---|---|
| | Avg | SD | fold | Z′ |
| 100% Proliferation (Cell Control) | 15453 | 1102 | 533 | 0.78 |
| 0% Proliferation (Media only) | 29 | 10 | | |

TABLE 45
Assay Drug Control
GDC-0941 @ 10 uM

| Avg | | % Inhibition | | |
|---|---|---|---|---|
| n1 | n2 | n1 | n2 | Avg |
| 6828 | 5808 | 56 | 63 | 59 |

TABLE 46
Assay Drug Control
Doxorubicin @ 10 uM

| Avg | | % Inhibition | | |
|---|---|---|---|---|
| n1 | n2 | n1 | n2 | Avg |
| 4686 | 4147 | 70 | 73 | 72 |

TABLE 47A
Assay Results

| | % Inhibition | | | | | | | |
|---|---|---|---|---|---|---|---|---|
| | 100 ug/ml | | 30 ug/ml | | | 10 ug/ml | | |
| Ayuflex ® | 33 | −11 | 11 | 0 | −54 | −66 | −43 | −39 |
| Capros ® | 9 | −19 | 46* | 19 | 27 | 25* | −7 | −2 |
| Phyto-BGS ® | 33 | 40 | 74* | 61 | 47 | 32 | 33 | 20 |
| Primavie ® | 60 | 51 | 61 | 67 | 35* | 73* | 21 | 28 |
| Sensoril ® | 71 | 67 | 82 | 68 | 57 | 69 | 51 | 17* |
| Ayuric ® | 41 | 20 | 41* | 8 | 1 | 52 | 60 | 36 |

TABLE 47B
Assay Results

| | 100 ug/ml | | 30 ug/ml | | 10 ug/ml | |
|---|---|---|---|---|---|---|
| | Avg % | | Avg % | | Avg % | |
| | inhibition | SD | inhibition | SD | inhibition | SD |
| Ayuflex ® | 33 | 0 | 6 | 8 | −41 | 3 |
| Capros ® | 9 | 0 | 23 | 6 | −5 | 4 |
| Phyto-BGS ® | 37 | 5 | 54 | 10 | 28 | 7 |
| Primavie ® | 56 | 7 | 64 | 4 | 25 | 5 |
| Sensoril ® | 69 | 2 | 69 | 13 | 60 | 13 |
| Ayuric ® | 30 | 15 | 5 | 5 | 44 | 11 |

As Phyto-BGS®, PrimaVie®, and Sensoril® showed the highest anti-cancer, anti-proliferative activity for AML PDCs of the extracts tested in FIG. 22, testing with the aim of revealing a dose-response curve and IC₅₀ for AML cancer cells was performed. FIG. 23 shows dose-response curves for Phyto-BGS®, PrimaVie®, and Sensoril® on AML cancer cells. An IC₅₀ of about 19.51 ug Sensoril®/ml was measured. Sensoril® showed the best anti-cancer activity of the 3 extracts tested. In a previous study, Sensoril® showed 60% inhibition at a 10 ug/ml dose, when tested at 3 graded doses. Tables 48-50 show data used to generate the dose-response curves.

TABLE 48
Dose-response curve
Phyto-BGS DRC

| | | | Raw data | | % Inhibition | | |
|---|---|---|---|---|---|---|---|
| Doses | Conc. | | n1 | n2 | n1 | n2 | Avg |
| 1 | 200 | ug/ml | 6031 | 8522 | 63 | 48 | 55 |
| 2 | 100 | ug/ml | 11057 | 14285 | 32 | 12 | 22 |
| 3 | 50 | ug/ml | 12265 | 15660 | 25 | 4 | 14 |
| 4 | 25 | ug/ml | 13697 | 16313 | 16 | 0 | 8 |
| 5 | 12.5 | ug/ml | 13577 | 16924 | 17 | −4 | 6 |
| 6 | 6.3 | ug/ml | 14206 | 17655 | 13 | −8 | 2 |
| 7 | 3.1 | ug/ml | 14013 | 15451 | 14 | 5 | 10 |
| 8 | 1.6 | ug/ml | 12746 | 14484 | 22 | 11 | 16 |

TABLE 49
Dose-response curve
Primavie DRC

| | | | Raw data | | % Inhibition | | |
|---|---|---|---|---|---|---|---|
| Doses | Conc. | | n1 | n2 | n1 | n2 | Avg |
| 1 | 200 | ug/ml | 11209 | 14738 | 31 | 10 | 20 |
| 2 | 100 | ug/ml | 14520 | 18950 | 11 | −16 | −3 |
| 3 | 50 | ug/ml | 13778 | 17755 | 15 | −9 | 3 |
| 4 | 25 | ug/ml | 14414 | 16829 | 12 | −3 | 4 |
| 5 | 12.5 | ug/ml | 14095 | 15965 | 14 | 2 | 8 |
| 6 | 6.3 | ug/ml | 13534 | 16727 | 17 | −3 | 7 |
| 7 | 3.1 | ug/ml | 13415 | 16638 | 18 | −2 | 8 |
| 8 | 1.6 | ug/ml | 15363 | 17617 | 6 | −8 | −1 |

TABLE 50
Dose-response curve
Sensoril DRC

| | | | Raw data | | % Inhibition | | |
|---|---|---|---|---|---|---|---|
| Doses | Conc. | | n1 | n2 | n1 | n2 | Avg |
| 1 | 200 | ug/ml | 238 | 344 | 99 | 98 | 98 |
| 2 | 100 | ug/ml | 474 | 740 | 97 | 96 | 96 |
| 3 | 50 | ug/ml | 3667 | 3860 | 78 | 76 | 77 |
| 4 | 25 | ug/ml | 6173 | 7312 | 62 | 55 | 59 |
| 5 | 12.5 | ug/ml | 8103 | 9989 | 50 | 39 | 45 |
| 6 | 6.3 | ug/ml | 10181 | 11777 | 38 | 28 | 33 |
| 7 | 3.1 | ug/ml | 12124 | 14101 | 26 | 14 | 20 |
| 8 | 1.6 | ug/ml | 13339 | 15317 | 18 | 6 | 12 |

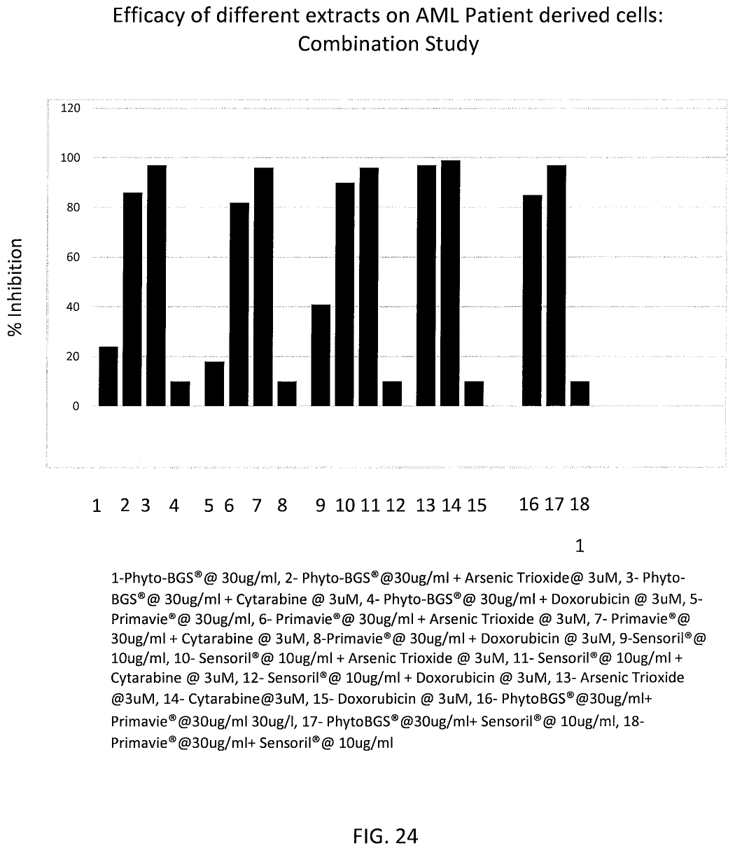

FIG. 24 represents information provided in Table 51. Phyto-BGS® (30 ug/ml) in combination with Arsenic Trioxide (3 uM), Cytarabine (3 uM), or Doxorubicin (3 uM), inhibited AML proliferation by 86%, 97%, and 100%, respectively. PrimaVie® (30 ug/ml) in combination with Arsenic Trioxide (3 uM), Cytarabine (3 uM), or Doxorubicin (3 uM), inhibited AML proliferation by 82%, 96%, and 100%, respectively. Sensoril® (10 ug/ml) in combination with Arsenic Trioxide (3 uM), Cytarabine (3 uM), or Doxorubicin (3 uM), inhibited AML proliferation by 90%, 96%, and 100%, respectively. The known anti-cancer drugs tested—Arsenic Trioxide, Cytarabine, and Doxorubicin, are shown as inhibiting AML proliferation by 97%, 99%, and 100%. Surprisingly, in combination with each other, the extracts tested performed well when compared the known anti-cancer drugs used as controls. The combination of Phyto-BGS (30 ug/ml) and PrimaVie® (30 ug/ml) inhibited AML cell proliferation by 85%; the Phyto-BGS (30 ug/ml) and Sensoril® (10 ug/ml) combination inhibited AML cell proliferation by 97%; and the PrimaVie® (30 ug/ml) and Sensoril® combination inhibited AML cell proliferation by 100%. In a previous study Phyto-BGS® (100 ug/ml) inhibited AML cell proliferation by 37%; PrimaVie® (100 ug/ml) inhibited AML cell proliferation by 56%; and Sensoril® (10 ug/ml) inhibited AML cell proliferation by 60%. Overall, in FIG. 24, extracts in combinations showed better anti-cancer effects than extracts alone. Known AML drugs cytarabine, doxorubicin, and arsenic trioxide (each at 3 uM) alone showed greater than 95% inhibition (97-100%), whereas combinations of extracts of the present invention showed AML cell proliferation inhibition at or greater than 85% (85-100%).

TABLE 51
Inhibition of AML proliferation

| | Raw data | | | % Inhibition | | | | |
|---|---|---|---|---|---|---|---|---|
| | n1 | n2 | n3 | n1 | n2 | n3 | Avg | SD |
| Phyto-BGS ®@ 30 ug/ml | 11231 | 13737 | 12743 | 35** | 21 | 27 | 24 | 4.0 |
| Phyto-BGS ®@30 ug/ml + | 1993 | 2545 | 2608 | 89 | 85 | 85 | 86 | 1.9 |
| Arsenic Trioxide@ 3 uM | | | | | | | | |
| Phyto-BGS ®@ 30 ug/ml + | 489 | 635 | 586 | 97 | 96 | 97 | 97 | 0.4 |
| Cytarabine @ 3 uM | | | | | | | | |
| Phyto-BGS ®@ 30 ug/ml + | 56 | 39 | 49 | 100 | 100 | 100 | 100 | 0.0 |
| Doxorubicin @ 3 uM | | | | | | | | |
| Primavie ®@ 30 ug/ml | 10627 | 13790 | 14651 | 39* | 21 | 16 | 18 | 3.5 |
| Primavie ®@ 30 ug/ml + | 2766 | 3463 | 3273 | 84 | 80 | 81 | 82 | 2.1 |
| Arsenic Trioxide @ 3 uM | | | | | | | | |
| Primavie ®@ 30 ug/ml + | 686 | 690 | 731 | 96 | 96 | 96 | 96 | 0.1 |
| Cytarabine @ 3 uM | | | | | | | | |
| Primavie ®@ 30 ug/ml + | 41 | 66 | 45 | 100 | 100 | 100 | 100 | 0.1 |
| Doxorubicin @ 3 uM | | | | | | | | |
| Sensoril ®@ 10 ug/ml | 7146 | 9782 | 10670 | 59* | 44 | 39 | 41 | 3.6 |
| Sensoril ®@ 10 ug/ml + | 1470 | 2007 | 1796 | 92 | 89 | 90 | 90 | 1.6 |
| Arsenic Trioxide @ 3 uM | | | | | | | | |
| Sensoril ®@ 10 ug/ml + | 588 | 731 | 835 | 97 | 96 | 95 | 96 | 0.7 |
| Cytarabine @ 3 uM | | | | | | | | |
| Sensoril ®@ 10 ug/ml + | 34 | 33 | 42 | 100 | 100 | 100 | 100 | 0.0 |
| Doxorubicin @ 3 uM | | | | | | | | |
| Arsenic Trioxide@ 3 uM | 551 | 599 | 484 | 97 | 97 | 97 | 97 | 0.3 |
| Cytarabine@ 3 uM | 203 | 192 | 140 | 99 | 99 | 99 | 99 | 0.2 |
| Doxorubicin@ 3 uM | 25 | 45 | 32 | 100 | 100 | 100 | 100 | 0.1 |
| PhytoBGS ®@30 ug/ml + | 2545 | 2857 | 2390 | 85 | 84 | 86 | 85 | 1.4 |
| Primavie ®@30 ug/ml 30 ug/l | | | | | | | | |
| PhytoBGS ®@30 ug/ml + | 557 | 583 | 534 | 97 | 97 | 97 | 97 | 0.1 |
| Sensoril ®@ 10 ug/ml | | | | | | | | |
| Primavie ®@30 ug/ml + | 36 | 37 | 39 | 100 | 100 | 100 | 100 | 0.0 |
| Sensoril ®@ 10 ug/ml | | | | | | | | |

Bolded text = Outliers

### Example II

### Inhibition of Acute Myeloid Leukemia Cell Proliferation by Sensoril®

AML (Acute myeloid leukemia) is one of the most common types of leukemia in adults. AML is an aggressive disease in which numerous myeloblasts are found in the bone marrow and blood of a subject. AML can spread to other parts of the body including the lymph nodes, liver, spleen, central nervous system, and testicles.

Several drugs have been approved to treat AML by the US Food and Drug Administration and are available to subjects in need, however, these drugs may have less than desired efficacy and may produce undesirable side, adverse, or toxic effects. Examples of drugs currently available to treat AML include arsenic trioxide (As₂O₃), cytarabine, and doxorubicin. Current therapeutic targets for treating AML include inhibitors of FLT3, IDH, histone deacetylase (HDAC), and BCL-2 polo-like kinase (Plk).

Anti-cancer luminescence assays on AML primary cells included incubation with extract for 72 hours, as in Example I. Doxorubicin was used as an assay control. In this Example, AML drugs were used as disease specific controls.

As discussed above in Example I, 6 extracts (Ayuflex®, Capros®, Phyto-BGS®, PrimaVie®, Sensoril®, and Ayuric®) were tested on AML PDC-SB cells (33866, M-4 type, 76 year old subject) at 3 concentrations: 100 ug/ml, 30 ug/ml, and 10 ug/ml. Three extracts (Phyto-BGS®, PrimaVie®, and Sensoril®) showed desirable activity in those tests and were further tested on the same PDCs (patient-derived cells) for determination of their IC₅₀ values and in combination with marketed AML drugs such as cytarabine, doxorubicin, or arsenic trioxide or in combination with other extracts. Sensoril® showed the best and most remarkable anti-cancer anti-proliferative activity alone with 60% inhibition at 10 ug/ml, tested at 3 graded doses, and with an IC₅₀ of about 20 ug/ml (See FIG. 23, showing an IC₅₀ of 19.51 ug/ml). The plate passed quality control criteria with a Z′ of 0.63. Extracts in combinations showed synergistic anti-cancer effects compared with extracts alone. AML drugs cytarabine, doxorubicin, and arsenic trioxide alone at 3 uM concentrations showed greater than 95% inhibition. See for instance FIGS. 1-6 and 22-24 and associated Tables and related discussion above. FIG. 22 shows anti-cancer effects of Phyto-BGS®), PrimaVie®, and Sensori)®; FIG. 23 shows dose-response curves for these 3 extracts on the AML PDCs, and FIG. 24 shows efficacy of the extracts and combinations as discussed above. These studied showed in part 37% inhibition by Phyto-BGS® (100 ug/ml), 56% inhibition by PrimaVie® (100 ug/ml), and 60% inhibition by Sensoril® (10 ug/ml).

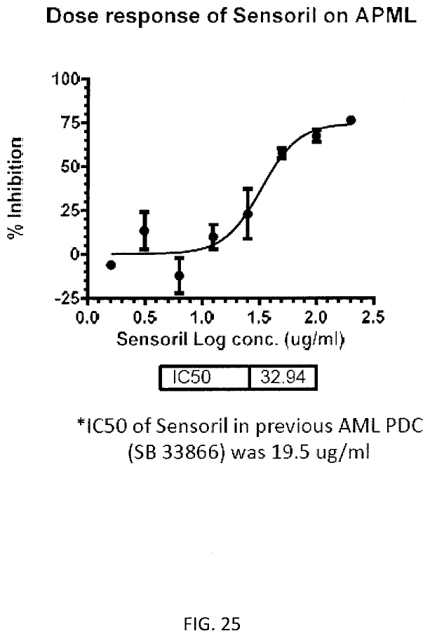

The data in Tables 52-59 and FIGS. 25-28 is from experiments using different AML PDCs than those discussed above to show Sensoril's effects on other PDCs of a different AML subtype. The AML PDCs evaluated below were an Acute Promyelocytic Leukemia (APL, APML) subtype, APL-SB 46120, from a 15-year-old human subject.

Sensoril® was evaluated at 3 different concentrations (IC₇₅, IC₅₀, and IC₂₅, derived from information such as shown in FIG. 23 and related information above), and also evaluated with IC₅₀ and IC₂₅ concentrations of marketed drugs, in part to show potential combinations that provide improved efficacy when administered to subjects. As the concentration of cytarabine, doxorubicin, and arsenic trioxide showed greater than 95% inhibition at 3 uM concentrations in Example I, lowering the concentrations of these drugs enabled a clearer showing of the synergistic effect of Sensoril®. In one experiment, the IC₅₀ and IC₂₅ of cytarabine, doxorubicin, and arsenic trioxide on the M4 APL cells discussed above and for instance in FIGS. 22-24 was determined. IC₂₅ values were calculated using IC₅₀ and hill slope. The IC₅₀ and IC₂₅ values of all three AML drugs on the M4 cells are described in Table 52. The drugs were used in these concentrations on the new APL subtype cells as described further below. Also, a dose-response curve for Sensoril® was generated in the new APL subtype PDCs, as shown in FIG. 25, and an IC₅₀ of 32.94 ug/ml was determined for the APL subtype PDCs. As a comparison and as noted above, the IC₅₀ for the M4 cells in previous studies was 19.5 ug/ml. Tables 53-57 present quality control and data analysis information relating to these experiments.

TABLE 52
Inhibitory Concentrations of Sensoril ® and AML drugs on AML cells

| Inhibitory | | | | |
|---|---|---|---|---|
| Concentration | | | | |
| (IC) | Sensoril ® | Cytarabine | As₂O₃ | Doxorubicin |
| IC₂₅ | 8.2 ug/ml | 23 nM | 1.38 uM | 40.7 nM |
| IC₅₀ | 19.5 ug/ml | 104 nM | 2.99 uM | 102.8 nM |
| IC₇₅ | 46.25 ug/ml | 470 nM | 6.45 uM | 260 nM |

TABLE 53
Plate 1 Quality Control
Analysis

| | | Standard | | |
|---|---|---|---|---|
| | Average | Deviation | Assay fold | Z′ |
| 100% Proliferation | 11591 | 1061 | 169 | 0.72 |
| (Cell Control) | | | | |
| 0% Proliferation | 69 | 5 | | |
| (Media only) | | | | |

TABLE 54
Plate 1 Quality Control (Doxorubicin)
Doxorubicin @ 10 uM

| | |
|---|---|
| Average | % Inhibition |

| n1 | n2 | n3 | n1 | n2 | n3 | Average |
|---|---|---|---|---|---|---|
| 465 | 486 | 627 | 97 | 96 | 95 | 96 |

TABLE 55
Plate 2 Quality Control
Analysis

| | | Standard | | |
|---|---|---|---|---|
| | Average | Deviation | Assay fold | Z′ |
| 100% Proliferation | 16734 | 1146 | 167 | 0.79 |
| (Cell Control) | | | | |
| 0% Proliferation | 101 | 35 | | |
| (Media only) | | | | |

TABLE 56
Plate 2 Quality Control (Doxorubicin)
Doxorubicin @ 10 uM

| | |
|---|---|
| Average | % Inhibition |

| n1 | n2 | n3 | n1 | n2 | n3 | Average |
|---|---|---|---|---|---|---|
| 418 | 668 | 621 | 98 | 97 | 97 | 97 |

TABLE 57
Plate 1 - Sensoril ® Dose-Response Curve
Sensoril ® Dose-Response Curve (DRC) (IC₅₀ = 32.94 ug/ml)

| | Concentration | Raw data | | Analysis | | | |
|---|---|---|---|---|---|---|---|
| Sample | ug/ml | n1 | n2 | n1 | n2 | Average | SD |
| 1 | 200 | 2617 | 2926 | 78 | 75 | 77 | 2 |
| 2 | 100 | 3579 | 4065 | 70 | 65 | 67 | 3 |
| 3 | 50 | 4661 | 5182 | 60 | 56 | 58 | 3 |
| 4 | 25 | 7733 | 10052 | 33 | 13 | 23 | 14 |
| 5 | 12.5 | 9878 | 11050 | 15 | 5 | 10 | 7 |
| 6 | 6.25 | 12217 | 13830 | −5 | −19 | −12 | 10 |
| 7 | 3.13 | 9193 | 10849 | 21 | 6 | 14 | 10 |
| 8 | 1.56 | 12252 | 14753 | −6 | −27 | −17 | 15 |

TABLE 58
Plate 1

| Combination | Avg | SD |
|---|---|---|
| Sensoril IC75 | 37 | 7 |
| Sensoril IC50 | −13 | 12 |
| Sensoril IC25 | −7 | 20 |
| Sensoril IC75 + Cytarabine IC50 | 36 | 4 |
| Sensoril IC50 + Cytarabine IC50 | −26 | 15 |
| Sensoril IC25 + Cytarabine IC50 | −20 | 27 |
| Sensoril IC75 + Cytarabine IC25 | 46 | 13 |
| Sensoril IC50 + Cytarabine IC25 | −19 | 15 |
| Sensoril IC25 + Cytarabine IC25 | −62 | 7 |
| Cytarabine IC50 | 20 | 16 |
| Cytarabine IC25 | −51 | 15 |
| Doxorubin @ 10 uM | 96 | 1 |

TABLE 59
Plate 2
Plate 2

| Combination | Avg | SD |
|---|---|---|
| Sensoril IC75 + As203 IC50 | 90 | 2 |
| Sensoril IC50 + As203 IC50 | 94 | 1 |
| Sensoril IC25 + As203 IC50 | 93 | 2 |
| Sensoril IC75 + As203 IC25 | 93 | 1 |
| Sensoril IC50 + As203 IC25 | 94 | 3 |
| Sensoril IC25 + As203 IC25 | 94 | 1 |
| Sensoril IC75 + Doxorubin IC50 | 93 | 3 |
| Sensoril IC50 + Doxorubin IC50 | 98 | 2 |
| Sensoril IC25 + Doxorubin IC50 | 98 | 1 |
| Sensoril IC75 + Doxorubin IC25 | 97 | 2 |
| Sensoril IC50 + Doxorubin IC25 | 93 | 1 |
| Sensoril IC25 + Doxorubin IC25 | 94 | 2 |
| AS203 IC50 | 60 | 9 |
| AS203 IC25 | 16 | 14 |
| Doxorubin IC50 | 43 | 18 |
| Doxorubin IC25 | −41 | 5 |
| Doxorubin 10 uM | 97 | 1 |

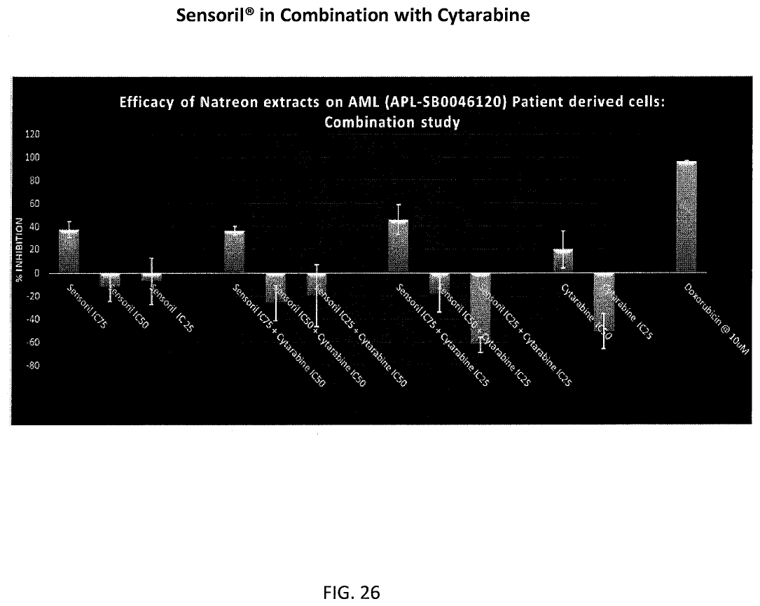

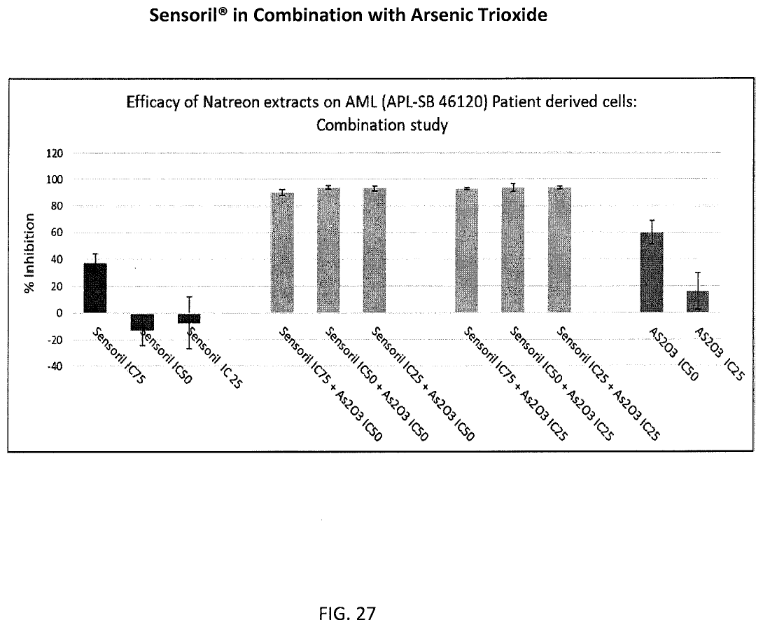

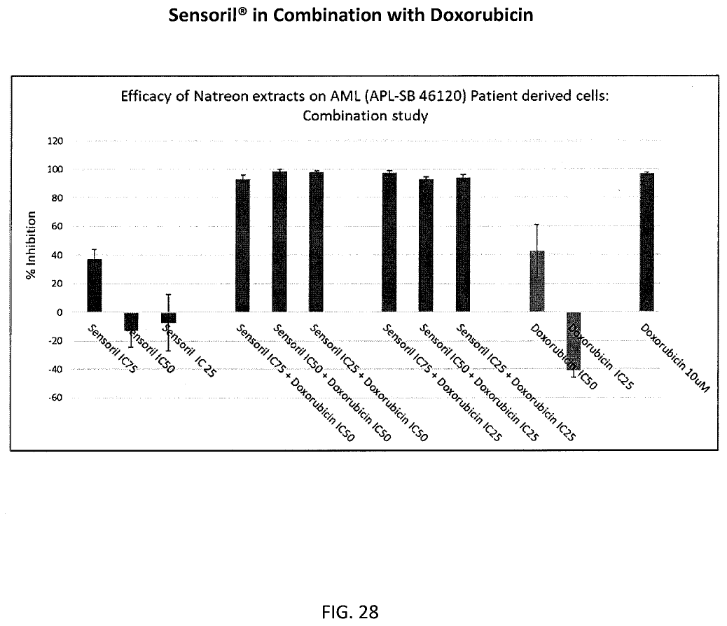

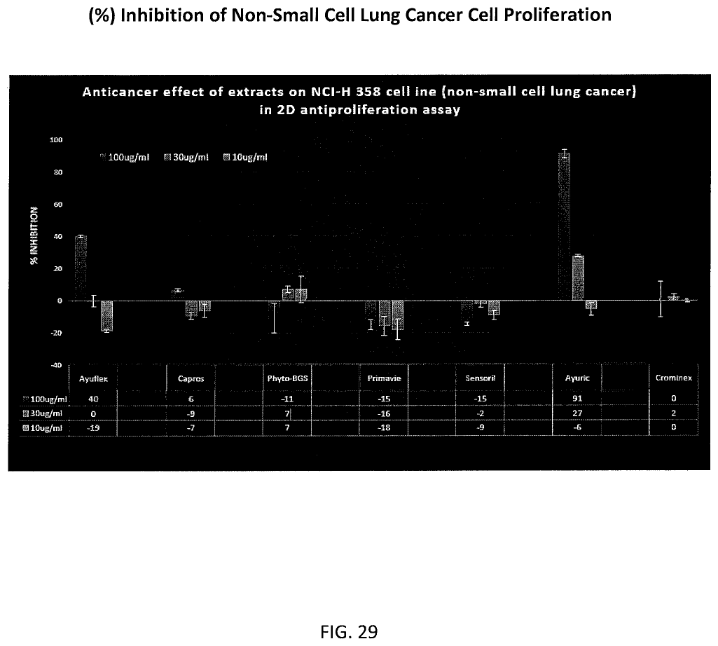

In FIGS. 26-28, Sensoril® was evaluated at 3 different concentrations alone (IC₇₅, IC₅₀, IC₂₅; Table 52), and combined with IC₅₀ and IC₂₅ concentrations of AML drugs (see also Tables 58-59). Sensoril® showed additive and synergistic anti-cancer anti-proliferative effects when combined with As₂O₃ (FIG. 27) and Doxorubicin (FIG. 28). Sensoril® showed similar IC₅₀ values for the AML subtype APL PDCs (approximately 31 ug/ml) and the AML (M4) PDCs of Example 1 (approximately 20 ug/ml). Arsenic trioxide and doxorubicin alone showed good efficacy compared with cytarabine alone. These results tally with clinical treatment, as arsenic trioxide and doxorubicin are preferred drugs for APL patients. In this study, Sensoril® in combination with cytarabine showed some but no significant inhibition of the subtype APL PDCs (FIG. 26).

Sensoril® shows potent anti-cancer anti-proliferative activity against different subtypes of AML, alone and in combination with drugs relevant to AML and APL treatment. Sensoril® may be administered with standard AML drugs, including at a low dose of the AML drugs, to achieve optimal efficacy and lower side effects of the drugs.

### Example III

### Inhibition of Non-Small Cell Lung Cancer Cell and Colon Cancer Cell Proliferation with Terminalia chebula (AyuFlex®) and Terminalia bellerica (Ayuric®)

Anti-proliferative and anti-cancer activity of extracts of this invention on non-small cell lung cancer cells and colon cancer cells were assessed generally as described in Example I and are discussed below. Terminalia chebula (AyuFlex®) and Terminalia bellerica (Ayuric®) showed significant activity. As noted throughout this application, the below data is intended to describe the present invention but not to be limiting.

Non-small cell lung cancer cells (cell line NCI-H-358) and colon cancer cells (cell line HT-29) were plated in a 384-well plate in amounts of about 1.25 k cells/well. AyuFlex®, Capros®, Phyto-BGS®, Primavie®, Sensoril®, Ayuric®, and Crominex® were added to the cells in concentrations of 100 ug/ml, 30 ug/ml, and 10 ug/ml, in triplicate, and incubated for 72 hours, and CellTiterGlo (Promega Corporation, Wisconsin, USA) used as a detection reagent, as described in Example I. Drugs used to treat non-small cell lung cancer (Standard of Care (SOC) drugs) Docetaxel (10 uM) and Doxorubicin (10 uM) were used as controls, as were drugs used to treat colon cancer (Standard of Care (SOC) drugs) 5-FU (5-fluoro-uracil, 10 uM) and Doxorubicin (10 uM). The IC₅₀ of docetaxel for non-small cell lung cancer H-358 cells has been published as 367 nM (Intl. J. Oncol. 31:241-252 (2007)). The IC₅₀ of 5-FU for colon cancer H-29 cells has been published as 13 uM (J. Surg. Res. 111(1):63-69 (2003)). Wells with untreated cells and with plain media were also included as controls. Plates passed quality control parameters.

### Anti-Cancer Anti-Proliferative Inhibition of Non-Small Cell Lung Cancer Cells

Tables 60-62 show data for controls, and FIG. 29 shows data relating to the 7 extracts tested (AyuFlex®, Capros®, Phyto-BGS®, Primavie®, Sensoril®, Ayuric®, and Crominex®), with bars reading left to right and the associated table reading top to bottom: 100 ug/ml, 30 ug/ml, and 10 ug/ml. Docetaxel exhibited 58% inhibition of non-small cell lung cancer cell proliferation at a 10 uM concentration. Assay control doxorubicin at 10 uM showed 78% inhibition of non-small cell lung cancer cell proliferation at a 10 uM concentration.

Of the 7 extracts tested and shown in FIG. 29 (left to right: Ayuflex®, Capros®, Phyto-BGS®, Primavie®, Sensoril®, Ayuric®, Crominex®, at 100, 30, and 10 ug/ml concentrations), Ayuric® showed the most potent anti-cancer and anti-proliferative effect, with 91% inhibition of proliferation at a 100 ug/ml concentration and 27% inhibition at 30 ug/ml. Ayuflex® also showed an anti-cancer anti-proliferative effect, inhibiting proliferation of non-small cell lung cancer cells by 40% at a 100 ug/ml concentration. The other extracts tested showed a lesser or no effect on cells of this lung cancer cell line.

TABLE 60
Plate Controls
Analysis

| | | Standard | | |
|---|---|---|---|---|
| | Average | Deviation | Assay fold | Z′ |
| 100% Proliferation | 62761 | 5395 | 1111 | 0.74 |
| (Cell Control) | | | | |
| 0% Proliferation | 57 | 18 | | |
| (Media only) | | | | |

TABLE 61
Plate Controls - Docetaxel
Docetaxel @ 10 uM

% Inhibition

| Average | | | | | | Average |
|---|---|---|---|---|---|---|
| n1 | n2 | n3 | n1 | n2 | n3 | (SD) |
| 23464 | 28364 | 26690 | 63 | 55 | 58 | 58(4) |

TABLE 62
Plate Controls - Doxorubicin
Doxorubicin @ 10 uM

% Inhibition

| Average | | | | | | Average |
|---|---|---|---|---|---|---|
| n1 | n2 | n3 | n1 | n2 | n3 | (SD) |
| 13222 | 14367 | 14575 | 79 | 77 | 77 | 78(1) |

### Anti-Cancer Anti-Proliferative Inhibition of Colon Cancer Cells

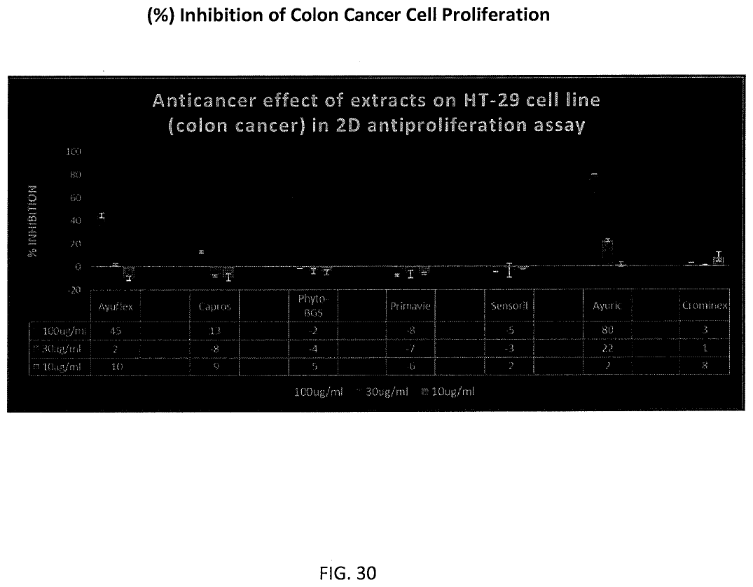

Tables 63-65 show data for controls and that the data passed quality control parameters. FIG. 30 shows data relating to the 7 extracts tested (AyuFlex®, Capros®, Phyto-BGS®, Primavie®, Sensoril®, Ayuric®, and Crominex®), with bars reading left to right and the associated table reading top to bottom: 100 ug/ml, 30 ug/ml, and 10 ug/ml. 5-FU provided only 12% inhibition of colon cancer cell proliferation at a 10 uM concentration. Assay control doxorubicin showed inhibition of colon cancer cell proliferation by 94%.

Of the 7 extracts tested, Ayuric® showed the most potent anti-cancer anti-proliferative activity, providing 80% inhibition of colon cancer cell proliferation at a concentration of 100 ug/ml and 22% inhibition at 30 ug/ml. Ayuflex® inhibited colon cancer cell proliferation by 45% at a 100 ug/ml concentration. The remaining 5 extracts showed little to no effect on this colon cancer cell line.

TABLE 63
Plate Controls
Analysis

| | | Standard | | |
|---|---|---|---|---|
| | Average | Deviation | Assay fold | Z′ |
| 100% Proliferation | 92463 | 2167 | 1988 | 0.93 |
| (Cell Control) | | | | |
| 0% Proliferation | 47 | 4 | | |
| (Media only) | | | | |

TABLE 64
Plate Controls - 5-FU
5-FU @ 10 uM

% Inhibition

| Average | | | | | | Average |
|---|---|---|---|---|---|---|
| n1 | n2 | n3 | n1 | n2 | n3 | (SD) |
| 74375 | 86123 | 83116 | 20 | 7 | 10 | 12(7) |

TABLE 65
Plate Controls - Doxorubicin
Doxorubicin @ 10 uM

% Inhibition

| Average | | | | | | Average |
|---|---|---|---|---|---|---|
| n1 | n2 | n3 | n1 | n2 | n3 | (SD) |
| 5430 | 6159 | 5802 | 94 | 93 | 94 | 94(0) |

### Example IV

### Inhibition of Glioma Cells with AyuFlex®, Capros®, and Ayuric®

As discussed with regard to FIG. 7 in Example I above, AyuFlex®, Capros®, and Ayuric® showed significant dose-dependent inhibition of patient derived primary glioma cells (described in Table 1). Also, as shown in FIG. 8, AyuFlex®, Capros®, and Ayuric® exhibited better inhibition than a standard-of-care drug, temozolomide, when tested alone or in combination. Combinations of these extracts (AyuFlex®+Capros®, AyuFlex®+Ayuric®, Capros®+Ayuric®) exhibited potent anti-cancer activity in the glioma PDCs, with >73% inhibition.

The anti-cancer activity of AyuFlex®, Capros®, and Ayuric® was further tested on two other glioma PDCs taken from 2-3 different subjects (SB 6129, SB 32833 (55 year old male)). These glioma PDCs had a different mix of cell types (neuronal, astrocytic, dendroglioma). Also, the anti-cancer activity of AyuFlex®, Capros®, and Ayuric® was further tested on existing glioblastoma cell line U87 MG, to allow comparisons with other compositions known in the art. AyuFlex®, Capros®, and/or Ayuric® were incubated with the new PDC lines and the U87 MG cell line for 72 hours, as described in Example I above.

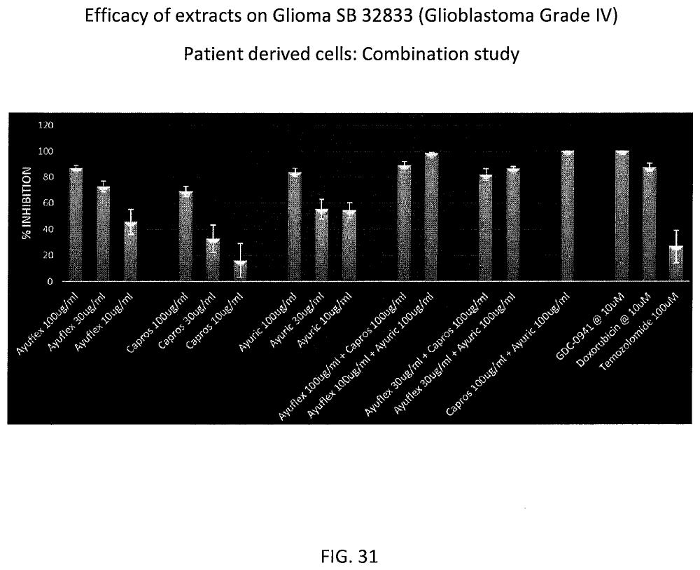

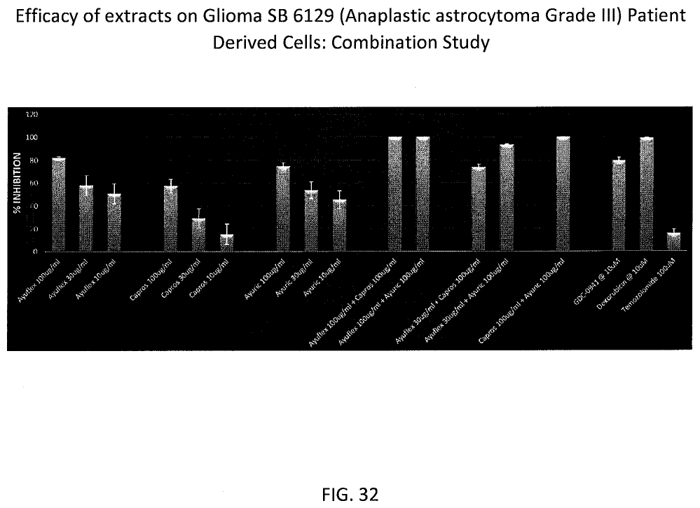

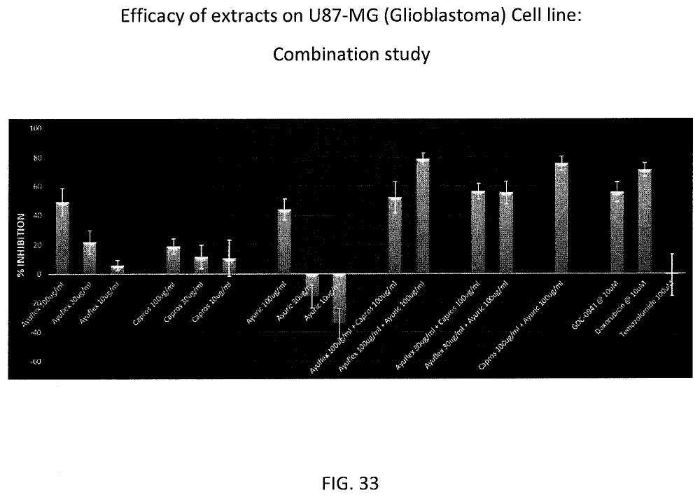

Tables 66-67 show data for controls and that the data passed quality control parameters. Table 68 relates to FIG. 31, a graph showing the efficacy of AyuFlex®, Capros®, and/or Ayuric® on Glioma SB 32833 Glioblastoma Grade IV patient derived cells, in a combination study. The graph shows, left to right, % inhibition of SB 32833 PDCs by AyuFlex® (100, 30, 10 ug/ml); Capros® (100, 30, 10 ug/ml); and Ayuric® (100, 30, 10 ug/ml); as well as combinations AyuFlex® (100 ug/ml)+Capros® (100 ug/ml), AyuFlex® (100 ug/ml)+Ayuric® (100 ug/ml), AyuFlex® (30 ug/ml)+Capros® (100 ug/ml), AyuFlex® (30 ug/ml)+Ayuric® (100 ug/ml), and Capros® (100 ug/ml)+Ayuric (100 ug/ml). (GDC-0941@10 uM, Doxorubicin@10 uM, and Temozolomide@100 uM shown at far right). FIG. 32 also shows the efficacy of the above extract doses (same as for FIG. 31) and combinations on glioma SB 6129 (anaplastic astrocytoma Grade III PDCs. FIG. 33 shows the efficacy of AyuFlex®, Capros®, and Ayuric® (same extract doses as FIG. 31) after a 72 hour incubation on known cell line U87-MG (glioblastoma) A dose-dependent effect was observed for all 3 extracts, with the anti-cancer effect on SB 32833 PDCs comparable to that described in Example 1 including FIGS. 7-8 above.

TABLE 66
Control data
Analysis

| | | Standard | | |
|---|---|---|---|---|
| | Average | Deviation | Assay fold | Z′ |
| 100% Proliferation | 39636 | 4762 | 855 | 0.64 |
| (Cell Control) | | | | |
| 0% Proliferation | 46 | 25 | | |
| (Media only) | | | | |

TABLE 67
Doxorubicin control
Doxorubicin @ 10 uM

% Inhibition

| Average | | | | | | Average |
|---|---|---|---|---|---|---|
| n1 | n2 | n3 | n1 | n2 | n3 | (SD) |
| 3578 | 5759 | 5708 | 91 | 86 | 86 | 87 |

TABLE 68
Inhibition of Glioma Cells

| | Raw data | | | % Inhibition | | | | |
|---|---|---|---|---|---|---|---|---|
| Combination | n1 | n2 | n3 | n1 | n2 | n3 | Avg | SD |
| Ayuflex 100 ug/ml | 4306 | 5649 | 5809 | 89 | 86 | 85 | 87 | 2 |
| Ayuflex 30 ug/ml | 8975 | 11126 | 12150 | 77 | 72 | 69 | 73 | 4 |
| Ayuflex 10 ug/ml | 17236 | 23956 | 23625 | 57 | 40 | 40 | 46 | 10 |
| Capros 100 ug/ml | 10643 | 13866 | 12564 | 73 | 65 | 68 | 69 | 4 |
| Capros 30 ug/ml | 21842 | 29142 | 29043 | 45 | 27 | 27 | 33 | 11 |
| Capros 10 ug/ml | 27354 | 36276 | 35965 | 31 | 8 | 9 | 16 | 13 |
| Ayuric 100 ug/ml | 5066 | 7275 | 7389 | 87 | 82 | 81 | 84 | 3 |
| Ayuric 30 ug/ml | 14419 | 20278 | 18373 | 64 | 49 | 54 | 55 | 8 |
| Ayuric 10 ug/ml | 15383 | 19439 | 19324 | 61 | 51 | 51 | 55 | 6 |
| Ayuflex 100 ug/ml + | 3083 | 4733 | 5300 | 92 | 88 | 87 | 89 | 3 |
| Capros 100 ug/ml | | | | | | | | |
| Ayuflex 100 ug/ml + | 474 | 1034 | 589 | 99 | 98 | 99 | 98 | 1 |
| Ayuric 100 ug/ml | | | | | | | | |
| Ayuflex 30 ug/ml + | 5109 | 8377 | 8192 | 87 | 79 | 79 | 82 | 5 |
| Capros 100 ug/ml | | | | | | | | |
| Ayuflex 30 ug/ml + | 4701 | 5847 | 5574 | 88 | 85 | 86 | 87 | 2 |
| Ayuric 100 ug/ml | | | | | | | | |
| Capros 100 ug/ml + | 15 | 15 | 20 | 100 | 100 | 100 | 100 | 0 |
| Ayuric 100 ug/ml | | | | | | | | |
| GDC-0941 @ 10 uM | 20 | 16 | 16 | 100 | 100 | 100 | 100 | 0 |
| Doxorubicin @ 10 uM | 3578 | 5759 | 5708 | 91 | 86 | 86 | 87 | 3 |
| Temozolomide @ 100 uM | 23511 | 31320 | 32454 | 41 | 21 | 18 | 27 | 12 |

In the three independent studies using 3 different gliomas (1 grade III and 2 grade IV gliomas), AyuFlex®, Capros®, and/or Ayuric® alone and in combination exhibited significant anti-cancer effects. Glioblastoma multiforme (GBM) is one of the most challenging brain tumor to treat, as patients generally do not live more than 1-2 years. Temozolomide is one of the only approved treatments, or the only treatment, for gliomas, however, 50-70% of patients treated with temozolomide have been reported as non-responders to temozolomide. Accordingly, the present invention includes treatment with AyuFlex®, Capros®, and/or Ayuric®, or other compositions of the present invention.

### Example V

### Inhibition of AML Cells with Sensoril®, Phyto-BGS, and PrimaVie®

As shown in FIGS. 22, 24, 26, 27, and 28, Sensoril®, Phyto-BGS® and PrimaVie® compositions of this invention inhibited 2 different types of AML PDCs. As shown in this Example, Sensoril®, Phyto-BGS®, and PrimaVie® compositions, and also a Crominex® composition, inhibited cancer cell proliferation in an AML cell line: HL60.

Inhibition assays were generally run as described for other AML assays above, with 5000 cells per well in 384 well plates. Standard-of-care drugs cytarabine (3 uM), arsenic trioxide (As₂O₃) (60 uM) and doxorubicin (10 uM) were assayed as controls. Phyto-GBS®, PrimaVie®, and Crominex® were assayed at 100 ug/ml, 30 ug/ml, and 10 ug/ml concentrations, and Sensoril® tested with a 9-point, 2-fold dose response curve (DRC), starting from 200 ug/ml. CellTiterGlo detection reagent was used, as discussed for instance in Example I above, and the 4 compositions were incubated with the cells for 72 hours. Tables 69-70 show data for controls and that the data passed quality control parameters.

TABLE 69
Assay controls—Plate 1
Analysis

| | | Standard | | |
|---|---|---|---|---|
| | Average | Deviation | Assay fold | Z′ |
| 100% Proliferation | 43535 | 2633 | 2561 | 0.82 |
| (Cell Control) | | | | |
| 0% Proliferation | 17 | 1 | | |
| (Media only) | | | | |

TABLE 70
Assay controls—Plate 2
Analysis

| | | Standard | | |
|---|---|---|---|---|
| | Average | Deviation | Assay fold | Z′ |
| 100% Proliferation | 87067 | 5601 | 189 | 0.81 |
| (Cell Control) | | | | |
| 0% Proliferation | 461 | 18 | | |
| (Media only) | | | | |

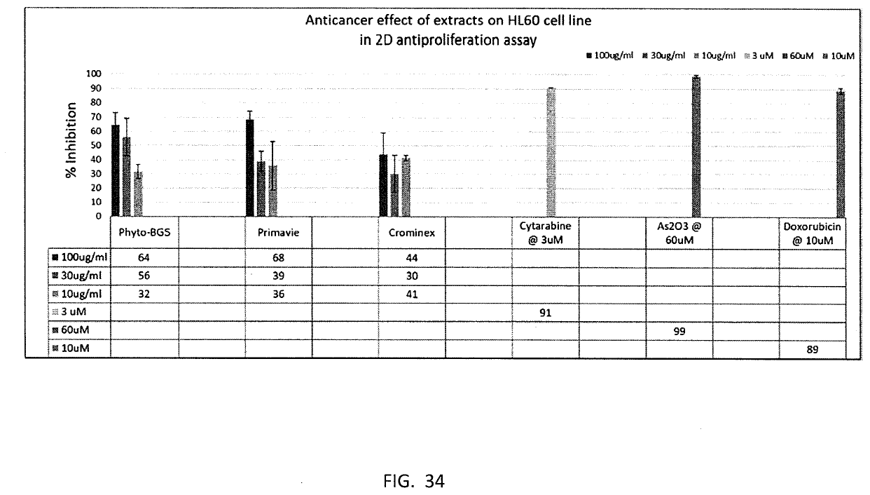

As shown in FIG. 34, Phyto-BGS® and PrimaVie® showed dose-dependent anti-cancer effects on HL60 cells, with greater than 60% inhibition at a dose of 100 ug/ml. Crominex® demonstrated 30-40% inhibition at all doses (100, 30, 10 ug/ml). Control standard-of-care drugs cytarabine (3 uM), As₂O₃ (60 uM), and doxorubicin (3 uM) exhibited 90% or higher inhibition of the HL60 AML cells.

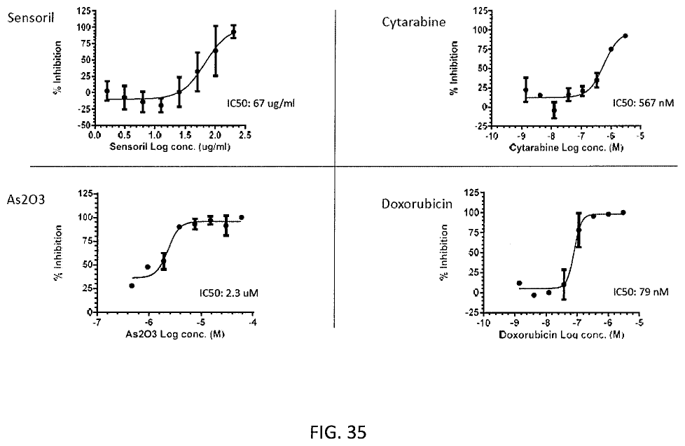

FIG. 35 shows dose-response curves of Sensoril®, cytarabine, AS2O3, and doxorubicin on the HL60 lines, with the dose-response curve for Sensoril® showing an IC₅₀ for inhibition of HL60 cells of 67 ug/ml; the curve for cytarabine showing an IC₅₀ of 567 nM; the curve for As₂O₃ showing an IC₅₀ of 2.3 uM; and the curve for doxorubicin showing an IC₅₀ of 79 nM. Combinations of Sensoril® with cytarabine, As2O3, and doxorubicin are expected to inhibit HL60 AML cells, for instance as shown in Table 71.

TABLE 71
Potential dosing for further experiments on HL60 AML cells

| | Sensoril ® | Cytarabine | As2O3 | Doxorubicin |
|---|---|---|---|---|
| | (ug/ml) | (uM) | (uM) | (uM) |
| IC75 | 109.65 | 1.08 | 3.01 | 0.106 |
| IC50 | 67.13 | 0.567 | 2.43 | 0.079 |
| IC25 | 41.10 | 0.298 | 1.72 | 0.059 |

### Example VI

### Inhibition of Triple Negative (TN) Breast Cancer PDCs (SB 30750) and TNBC Cell Line MDAMB-231 with AyuFlex®, Capros®, and Ayuric®

As shown for instance in FIGS. 16-18, AyuFlex®, Capros®, and Ayuric® showed exceptional anti-cancer effects against SB 36344 cells. AyuFlex® (10 ug/ml) was previously shown to inhibit TN breast cancer PDCs by 54%; Capros® (30 ug/ml), by 58%; and Ayuric® (30 ug/ml) by 47%.

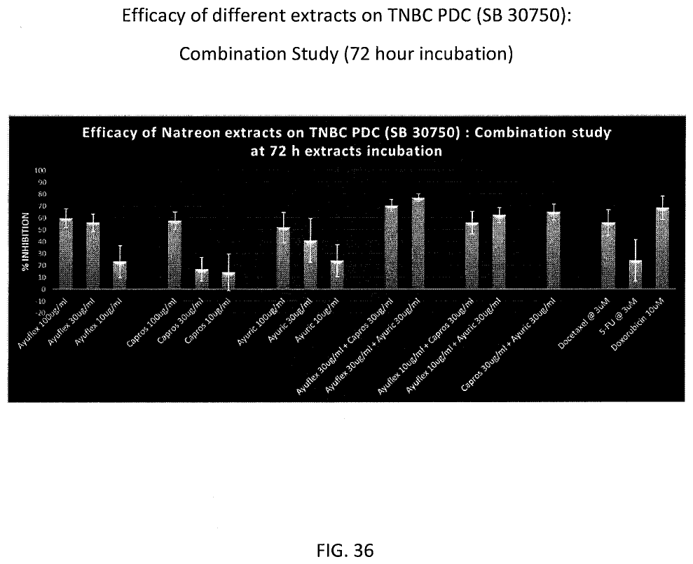

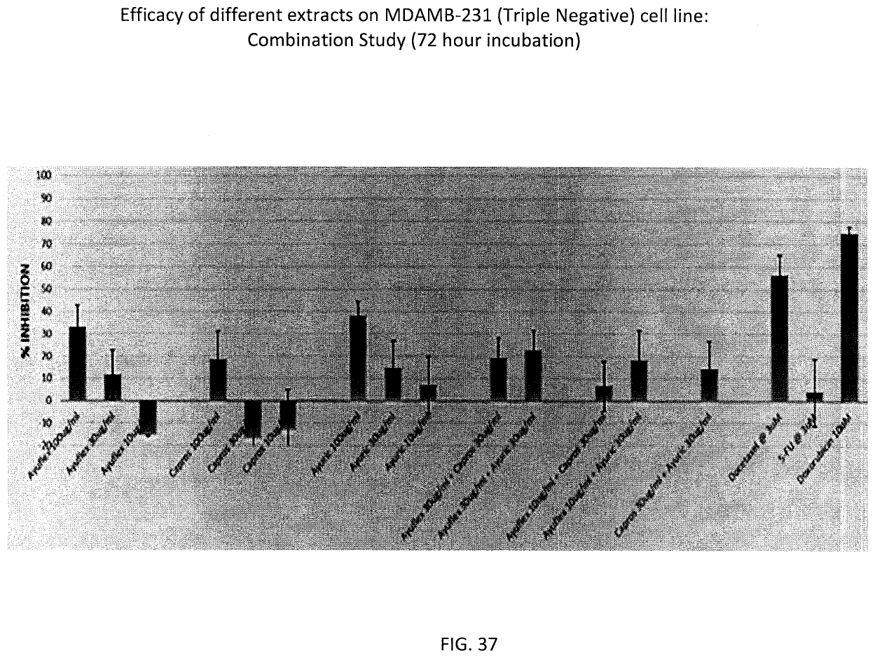

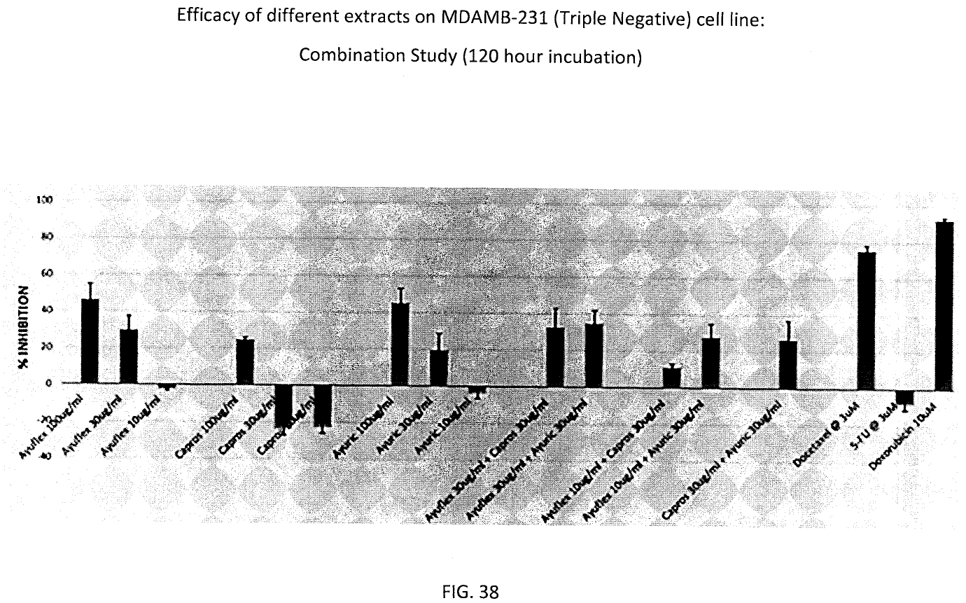

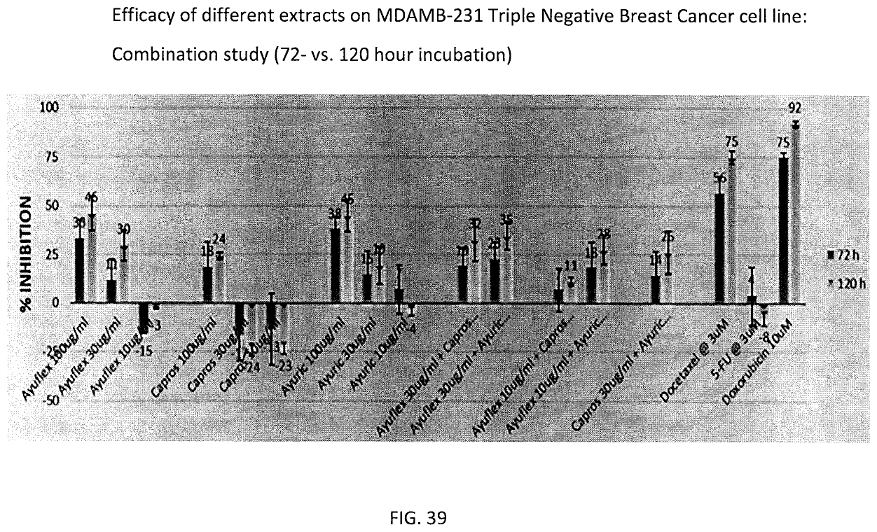

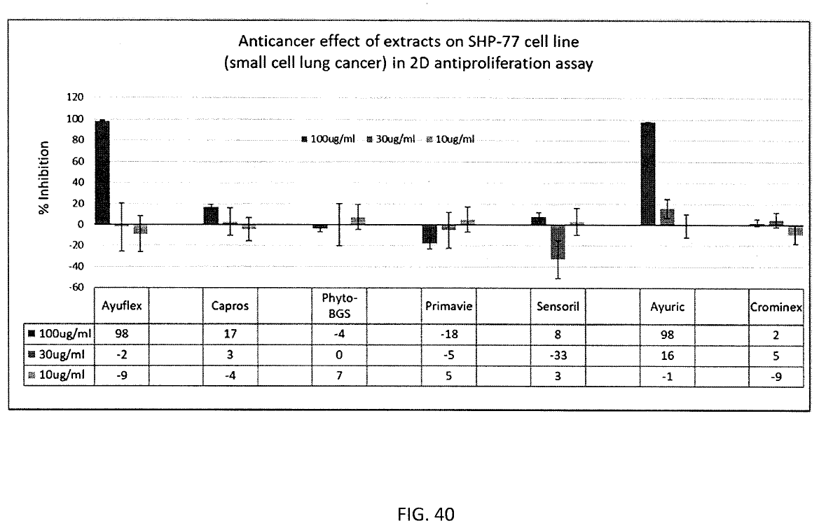

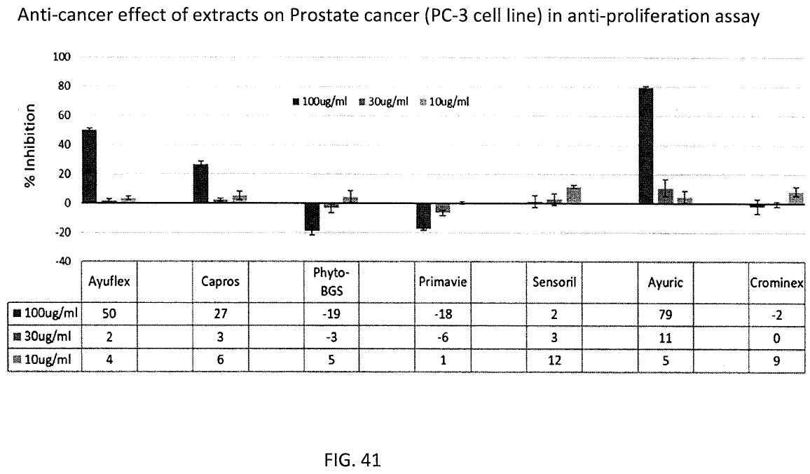

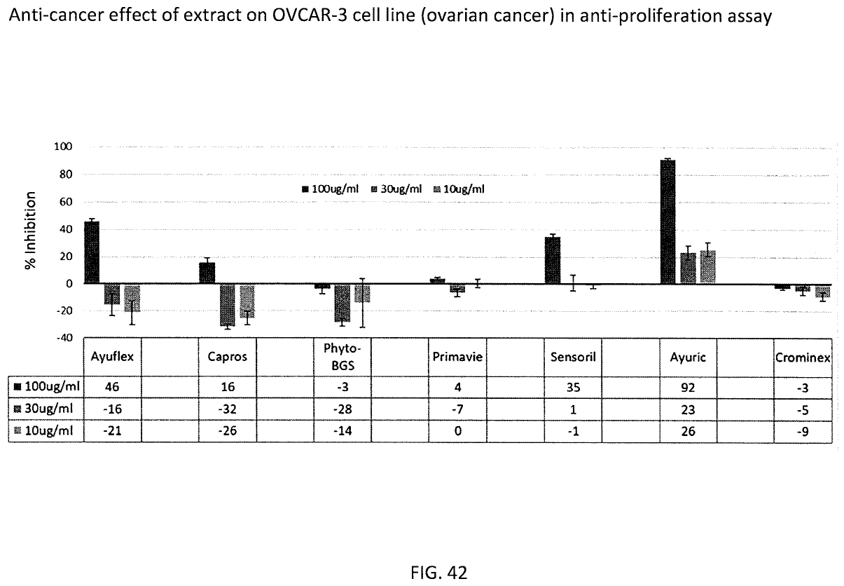

In this Example, AyuFlex®, Capros®, and Ayuric® were each applied to different PDCs than previous tests, TN breast cancer PDCs SB 30750, and tested via the anti-proliferation assay described above. As shown in FIG. 36, each of AyuFlex®, Capros®, and Ayuric® was applied to the SB 30750 PDCs alone in concentrations of 100, 30, and 10 ug/ml, and combined as follows: AyuFlex® (30 ug/ml)+Capros® (30 ug/ml), AyuFlex® (30 ug/ml)+Ayuric® (30 ug/ml), AyuFlex® (10 ug/ml)+Capros® (30 ug/ml), AyuFlex® (10 ug/ml)+Ayuric® (30 ug/ml), and Capros® (30 ug/ml)+Ayuric® (30 ug/ml). Docetaxel (3 uM), 5-FU (3 uM), and doxorubicin (10 uM) controls are shown at the far right of the Figure. Incubation times were 72 hours. AyuFlex®, Capros®, and Ayuric® each showed a dose-dependent effect in the SB 30750 PDCs, and showed similar anti-cancer, inhibitory effects to the inhibition seen in the PDC cells of a different subject (SB 36344) tested in FIGS. 16 and 18.

Also, AyuFlex®, Capros®, and Ayuric® were tested in the anti-proliferative assay discussed above in the TN Breast Cancer cell line MDAMB-231. AyuFlex®, Capros®, and Ayuric® were incubated for 72 hours and 120 hours. The dose-response efficacy of AyuFlex®, Capros®, and Ayuric® after a 72-hour period of incubation was similar to that observed after a 120-hour period of incubation with the MDAMB-231 cells. Without being bound by theory, possibly this was due to saturation of cell growth at the 120-hour time-point.

Surprisingly, AyuFlex®, Capros®, and Ayuric® extracts showed better activity in both PDCs than in the MDAMB-231 cell line. The MDAMB-231 cell line was established from pleural effusion of a Caucasian female with a metastatic mammary adenocarcinoma 1, and has a high stem cell percentage. The TNBC PDCs used in earlier studies (shown in FIGS. 16-18) were from Grade II and TNM-T2N1aMx tumor, and the PDCs of the present Example (SB 30750) are from a Grade I and TNM-T2N1aMx tumor, both likely lower grade and non-metastatic tumors compared with MDAMB-231 cells. Without being bound by theory, it may be that the extracts are more active in earlier stage tumors than metastatic ones.

In summary, with the results of this Example VI and previous studies described herein, AyuFlex®, Capros®, and Ayuric® extracts demonstrated reproducible anti-cancer effects in 3 independent experiments using two different TNVC cancer patient derived cells.

TABLE 72
Plate Controls
Analysis

| | | Standard | | |
|---|---|---|---|---|
| | Average | Deviation | Assay fold | Z′ |
| 100% Proliferation | 74408 | 9061 | 1413 | 0.63 |
| (Cell Control) | | | | |
| 0% Proliferation | 53 | 6 | | |
| (Media only) | | | | |

TABLE 73
Doxorubicin control
Doxorubicin @ 10 uM

% Inhibition

| Average | | | | | | Average |
|---|---|---|---|---|---|---|
| n1 | n2 | n3 | n1 | n2 | n3 | (SD) |
| 16406 | 23949 | 31038 | 78 | 68 | 58 | 68 |

TABLE 74
Inhibition of TNBC PDCs (SB 30750) by AyuFlex ®, Capros ®, and Ayuric ®

| | Raw data | | | % Inhibition | | | | |
|---|---|---|---|---|---|---|---|---|
| Combination | n1 | n2 | n3 | n1 | n2 | n3 | Avg | SD |
| Ayuflex 100 ug/ml | 23242 | 33738 | 33268 | 69 | 55 | 55 | 60 | 8 |
| Ayuflex 30 ug/ml | 27053 | 33894 | 37482 | 64 | 54 | 50 | 56 | 7 |
| Ayuflex 10 ug/ml | 45510 | 63323 | 62574 | 39 | 15 | 16 | 23 | 14 |
| Capros 100 ug/ml | 25381 | 34302 | 35137 | 66 | 54 | 53 | 58 | 7 |
| Capros 30 ug/ml | 54237 | 63005 | 68764 | 27 | 15 | 8 | 17 | 10 |
| Capros 10 ug/ml | 50700 | 70923 | 69595 | 32 | 5 | 6 | 14 | 15 |
| Ayuric 100 ug/ml | 25631 | 37388 | 44444 | 66 | 50 | 40 | 52 | 13 |
| Ayuric 30 ug/ml | 30833 | 42730 | 58052 | 59 | 43 | 22 | 41 | 18 |
| Ayuric 10 ug/ml | 45673 | 59207 | 64851 | 39 | 20 | 13 | 24 | 13 |
| Ayuflex 30 ug/ml + | 18056 | 22928 | 25595 | 76 | 69 | 66 | 70 | 5 |
| Capros 30 ug/ml | | | | | | | | |
| Ayuflex 30 ug/ml + | 14857 | 17538 | 19683 | 80 | 76 | 74 | 77 | 3 |
| Ayuric 30 ug/ml | | | | | | | | |
| Ayuflex 10 ug/ml + | 25519 | 33410 | 39663 | 66 | 55 | 47 | 56 | 10 |
| Capros 30 ug/ml | | | | | | | | |
| Ayuflex 10 ug/ml + | 22904 | 30026 | 31629 | 69 | 60 | 58 | 62 | 6 |
| Ayuric 30 ug/ml | | | | | | | | |
| Capros 30 ug/ml + | 20621 | 29043 | 29474 | 72 | 61 | 60 | 65 | 7 |
| Ayuric 30 ug/ml | | | | | | | | |
| Docetaxel @ 3 uM | 24001 | 35688 | 39462 | 68 | 52 | 47 | 56 | 11 |
| 5-FU @ 3 uM | 41465 | 63866 | 64261 | 44 | 14 | 14 | 24 | 18 |
| Doxorubicin @ 10 uM | 16406 | 23949 | 31038 | 78 | 68 | 58 | 68 | 10 |

TABLE 75
Inhibition of MDAMB-231 cell line by AyuFlex ®, Capros ®, and
Ayuric ® (72 hour incubation)

| | Raw data | | | % Inhibition | | | | |
|---|---|---|---|---|---|---|---|---|
| Combination | n1 | n2 | n3 | n1 | n2 | n3 | Avg | SD |
| Ayuflex 100 ug/ml | 58945 | 77704 | 76140 | 44 | 27 | 28 | 33 | 10 |
| Ayuflex 30 ug/ml | 80393 | 98268 | 102334 | 24 | 7 | 3 | 11 | 11 |
| Ayuflex 10 ug/ml | 92286 | 122115 | 122215 | 13* | −15 | −16 | −15 | 0 |
| Capros 100 ug/ml | 70641 | 92822 | 95695 | 33 | 12 | 10 | 18 | 13 |
| Capros 30 ug/ml | 108078 | 130370 | 132500 | −2 | −23 | −25 | −17 | 13 |
| Capros 10 ug/ml | 97875 | 128124 | 134081 | 7 | −21 | −27 | −13 | 18 |
| Ayuric 100 ug/ml | 57505 | 70158 | 69844 | 46 | 34 | 34 | 38 | 7 |
| Ayuric 30 ug/ml | 75642 | 98513 | 97248 | 29 | 7 | 8 | 15 | 12 |
| Ayuric 10 ug/ml | 83078 | 106876 | 105518 | 21 | −1 | 0 | 7 | 13 |
| Ayuflex 30 ug/ml + | 74411 | 92848 | 90342 | 30 | 12 | 15 | 19 | 9 |
| Capros 30 ug/ml | | | | | | | | |
| Ayuflex 30 ug/ml + | 70949 | 87917 | 86978 | 33 | 17 | 18 | 23 | 9 |
| Ayuric 30 ug/ml | | | | | | | | |
| Ayuflex 10 ug/ml + | 85852 | 108852 | 101693 | 19 | −3 | 4 | 7 | 11 |
| Capros 30 ug/ml | | | | | | | | |
| Ayuflex 10 ug/ml + | 70288 | 95788 | 94340 | 34 | 9 | 11 | 18 | 14 |
| Ayuric 30 ug/ml | | | | | | | | |
| Capros 30 ug/ml + | 75562 | 97654 | 99317 | 29 | 8 | 6 | 14 | 13 |
| Ayuric 30 ug/ml | | | | | | | | |
| Docetaxel @ 3 uM | 35229 | 52837 | 50750 | 67 | 50 | 52 | 56 | 9 |
| 5-FU @ 3 uM | 83364 | 111466 | 110358 | 21 | −5 | −4 | 4 | 15 |
| Doxorubicin 10 uM | 23120 | 28746 | 28430 | 78 | 73 | 73 | 75 | 3 |

TABLE 76
Inhibition of MDAMB-231 cell line by AyuFlex ®, Capros ®, and
Ayuric ® (120 hour incubation)

| | Raw data | | | % Inhibition | | | | |
|---|---|---|---|---|---|---|---|---|
| Combination | n1 | n2 | n3 | n1 | n2 | n3 | Avg | SD |
| Ayuflex 100 ug/ml | 56389 | 77349 | 72974 | 56 | 39 | 43 | 46 | 9 |
| Ayuflex 30 ug/ml | 78537 | 95588 | 95641 | 39 | 25 | 25 | 30 | 8 |
| Ayuflex 10 ug/ml | 95138 | 130502 | 131567 | 25* | −2 | −3 | −3 | 1 |
| Capros 100 ug/ml | 70583 | 95001 | 98086 | 45* | 26 | 23 | 24 | 2 |
| Capros 30 ug/ml | 125560 | 155502 | 161033 | 2* | −22 | −26 | −24 | 3 |
| Capros 10 ug/ml | 122241 | 159839 | 154609 | 4* | −25 | −21 | −23 | 3 |
| Ayuric 100 ug/ml | 58558 | 77747 | 74476 | 54 | 39 | 42 | 45 | 8 |
| Ayuric 30 ug/ml | 89163 | 111142 | 108375 | 30 | 13 | 15 | 19 | 9 |
| Ayuric 10 ug/ml | 102684 | 130666 | 135088 | 20* | −2 | −6 | −4 | 2 |
| Ayuflex 30 ug/ml + | 71269 | 97666 | 90166 | 44 | 24 | 29 | 32 | 11 |
| Capros 30 ug/ml | | | | | | | | |
| Ayuflex 30 ug/ml + | 72855 | 89938 | 87457 | 43 | 30 | 32 | 35 | 7 |
| Ayuric 30 ug/ml | | | | | | | | |
| Ayuflex 10 ug/ml + | 88342 | 111648 | 115247 | 31* | 13 | 10 | 11 | 2 |
| Capros 30 ug/ml | | | | | | | | |
| Ayuflex 10 ug/ml + | 81695 | 99895 | 96079 | 36 | 22 | 25 | 28 | 8 |
| Ayuric 30 ug/ml | | | | | | | | |
| Capros 30 ug/ml + | 78312 | 100528 | 103458 | 39 | 21 | 19 | 26 | 11 |
| Ayuric 30 ug/ml | | | | | | | | |
| Docetaxel @ 3 uM | 28378 | 36725 | 30693 | 78 | 71 | 76 | 75 | 3 |
| 5-FU @ 3 uM | 102508 | 140779 | 134513 | 20* | −10 | −5 | −8 | 3 |
| Doxorubicin 10 uM | 7939 | 11844 | 10217 | 94 | 91 | 92 | 92 | 2 |

### Example VII

### Inhibition of Small Cell Lung Cancer Cells with Different Extracts

SHP-77 (small cell lung cancer) cells were incubated for 72 hours with 100 ug/ml, 30 ug/ml, and 10 ug/ml each of AyuFlex®, Capros® Phyto-BGS®, PrimaVie®, Sensoril®, Ayuric®, and Crominex®, and tested according to the anti-proliferative assay described above. Cells were plated at 1.25 k/well in 384 well plates. Standard of care drug docetaxel was also tested at a 10 uM concentration as a control. Anti-proliferative assays were generally performed as discussed above, in triplicate, with detection reagent CellTiterGlo® as discussed for instance in Example I. Tables 77-79 show data for controls and that the data passed quality control parameters.

TABLE 77
Plate Controls
Analysis

| | | Standard | | |
|---|---|---|---|---|
| | Average | Deviation | Assay fold | Z′ |
| 100% Proliferation | 49047 | 3203 | 30 | 0.79 |
| (Cell Control) | | | | |
| 0% Proliferation | 1613 | 79 | | |
| (Media only) | | | | |

TABLE 78
Docetaxel control
Docetaxel @ 10 uM

% Inhibition

| Average | | | | | | Average |
|---|---|---|---|---|---|---|
| n1 | n2 | n3 | n1 | n2 | n3 | (SD) |
| 6365 | 5908 | 6786 | 90 | 91 | 89 | 90(1) |

TABLE 79
Doxorubicin control
Doxorubicin @ 10 uM

% Inhibition

| Average | | | | | | Average |
|---|---|---|---|---|---|---|
| n1 | n2 | n3 | n1 | n2 | n3 | (SD) |
| 4634 | 5086 | 5168 | 94 | 93 | 93 | 93(1) |

Docetaxel, a drug currently used for the treatment of small cell lung cancer, exhibited 90% inhibition of cell proliferation at the 10 uM concentration tested. The assay control, doxorubicin, exhibited 93% inhibition at the 10 uM concentration tested.

Of the extracts tested, AyuFlex® and Ayuric® exhibited the most potent anti-cancer effect on the SHP-77 small cell lung cancer cell line, exhibiting 98% inhibition at 100 ug/ml. The anti-cancer activity of the extracts on SHP-77 cells is similar to that of H-358 cells (non-small cell cancer, NSCLC, shown in Example III), with Ayuric® and AyuFlex® also showing the best anti-cancer effect in those cells.

### Example VIII

Prostate cancer (PC-3) cells and Ovarian cancer (OVCAR-3) cells were incubated for 72 hours with 100 ug/ml, 30 ug/ml, and 10 ug/ml each of AyuFlex®, Capros® Phyto-BGS®, PrimaVie®, Sensoril®, Ayuric®, and Crominex®, and tested according to the anti-proliferative assay described above. Cells were plated at 1.25 k/well in 384 well plates. Standard of care drug docetaxel and control doxorubicin were each also tested at a 10 uM concentration. Anti-proliferative assays were generally performed as discussed in Example I above, in triplicate, with detection reagent CellTiterGlo. Tables 80-82 show data for controls for assays on prostate cancer (PC-3 cells), and that the data passed quality control parameters. Tables 83-85 show data for controls for assays on ovarian cancer (OVCAR-3 cells), and that the data passed quality control parameters.

TABLE 80
Plate Controls
Analysis

| | | Standard | | |
|---|---|---|---|---|
| | Average | Deviation | Assay fold | Z′ |
| 100% Proliferation | 54196 | 3632 | 415 | 0.80 |
| (Cell Control) | | | | |
| 0% Proliferation | 131 | 37 | | |
| (Media only) | | | | |

TABLE 81
Docetaxel control
Docetaxel @ 10 uM

% Inhibition

| Average | | | | | | Average |
|---|---|---|---|---|---|---|
| n1 | n2 | n3 | n1 | n2 | n3 | (SD) |
| 25757 | 18168 | 24121 | 53 | 67 | 56 | 58(7) |

TABLE 82
Doxorubicin control
Doxorubicin @ 10 uM

% Inhibition

| Average | | | | | | Average |
|---|---|---|---|---|---|---|
| n1 | n2 | n3 | n1 | n2 | n3 | (SD) |
| 16210 | 16521 | 15937 | 70 | 70 | 71 | 70(1) |

TABLE 83
Plate Controls
Analysis

| | | Standard | | |
|---|---|---|---|---|
| | Average | Deviation | Assay fold | Z′ |
| 100% Proliferation | 45784 | 1777 | 595 | 0.88 |
| (Cell Control) | | | | |
| 0% Proliferation | 77 | 6 | | |
| (Media only) | | | | |

TABLE 84
Docetaxel control
Docetaxel @ 10 uM

% Inhibition

| Average | | | | | | Average |
|---|---|---|---|---|---|---|
| n1 | n2 | n3 | n1 | n2 | n3 | (SD) |
| 16072 | 14601 | 15965 | 65 | 68 | 65 | 66(2) |

TABLE 85
Doxorubicin control
Doxorubicin @ 10 uM

% Inhibition

| Average | | | | | | Average |
|---|---|---|---|---|---|---|
| n1 | n2 | n3 | n1 | n2 | n3 | (SD) |
| 12071 | 11517 | 9891 | 74 | 75 | 79 | 76(2) |

With regard to prostate cancer (PC-3) cells, docetaxel, a drug currently used for the treatment of prostate cancer, exhibited 58% inhibition of cell proliferation at the 10 uM concentration tested. The assay control, doxorubicin, exhibited 70% inhibition at the 10 uM concentration tested.

Of the extracts tested, Ayuric® exhibited the most potent anti-cancer effect on the prostate cancer (PC-3) cell line, exhibiting 79% inhibition at 100 ug/ml; followed by AyuFlex®, which exhibited 50% inhibition in the assay at 100 ug/ml.

With regard to ovarian cancer (OVCAR-3) cells, docetaxel, a drug currently used for the treatment of ovarian cancer, exhibited 66% inhibition of cell proliferation at the 10 uM concentration tested. The assay control, doxorubicin, exhibited 76% inhibition at the 10 uM concentration tested.

Of the extracts tested, Ayuric® exhibited the most potent anti-cancer effect on the ovarian cancer cell line, exhibiting 92% inhibition of cell proliferation at 100 ug/ml, and 23% inhibition at 30 ug/ml. The next effective extracts against the proliferation of the OVCAR-3 ovarian cancer cells were AyuFlex® and Sensoril®, exhibiting 46% and 35% inhibition respectively at 100 ug/ml.

### Example IX

### Inhibition of AML (HL60 Cell Line) Cancer Cells with Hydroethanolic Extracts of Withania somnifera (Sensoril®-AWE)

Hydroethanolic extracts of Withania somnifera (Sensoril®-AWE) showed significant anti-cancer effects in AML cancer cells of the HL60 cell line. 2500 cells were plated per well in 384 well plates. Standard of care drug doxorubicin was included as a control at 10 uM. 6 different samples of Sensoril®-AWE, prepared with hydroethanolic extraction were tested in the anti-proliferative assay as generally described in Example I above. Extracts were prepared at 2 mg/ml concentrations in view of increased solubility and tested at 400 ug/ml, 2-fold serial dilutions, to prepare an 8-point Dose-Response Curve. CellTiter Glo was used as a detection reagent. Extracts were incubated with the AML HL60 cells for 72 hours. Table 86 provides information on hydroethanolic extract quantification and markers according to this invention. Full analysis of some Samples described below are as follows: Lot #WS09120: 19.34% w/w Withanolide glycosides, 7.52% w/w Withanolide Aglycones (as Withaferin A), 34.42% w/w Oligosaccharides; Lot #WS09820: 13.16% w/w Withanolide glycosides, 2.60% w/w Withanolide Aglycones (as Withaferin A), 26.52% w/w Oligosaccharides; Lot #WS09920: 12.68% w/w Withanolide glycosides, 2.71% w/w Withanolide Aglycones (as Withaferin A), 23.55% w/w Oligosaccharides; Lot #WS10020: 12.20% w/w Withanolide glycosides, 2.39% w/w Withanolide Aglycones (as Withaferin A), 24.64% w/w Oligosaccharides. Tables 87-90 show data for controls and that the data passed quality control parameters.

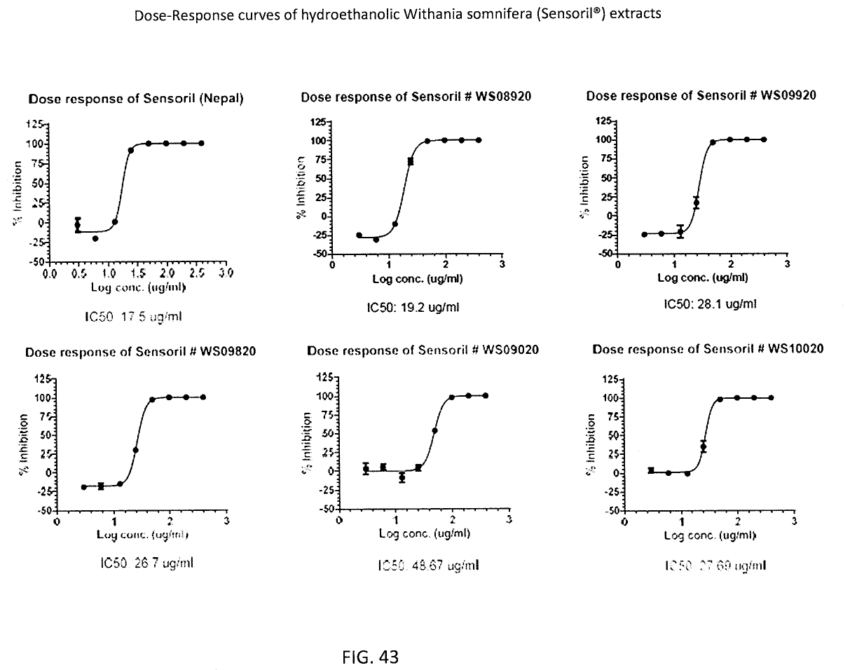

All hydroethanolic samples of Withania somnifera (Sensoril®-AWE) showed a sigmoidal saturated IC₅₀ dose-response curve with significant inhibition of AML HL60 cell proliferation. The most potent sample was the “Nepal” sample (Sample 1 of Table 91 below, Lot #WS09120), which showed an IC₅₀ of 17.5 ug/ml, with Sample 2, based solely on Withania somnifera leaves, following closely with an IC₅₀ of 19.2 ug/ml. Sample 3, 4, and 6 showed higher IC₅₀ values, ranging from 26-28 ug/ml, and Sample 5 showed lowest activity with an IC₅₀ of 48.7 ug/ml. See Table 91 for further information on Sample preparation and a listing of IC₅₀ s found for each Sample, Tables 92-97 for experimental data relating to the Samples, and FIG. 43 for relevant dose-response curves.

TABLE 86
Withania somnifera (Ashwagandha) hydroethanolic extracts (Sensoril ®-AWE) of the present invention

| | | | Quantification using | | | |
|---|---|---|---|---|---|---|
| | | | Withanoside-IV and | | Quantification using | |
| | | | Withanolide-A as Markers | | individual markers | |
| | | | | Total | | Total |
| Sample name | Class of | | Content | Content | Content | Content |
| (Lot#) | analytes | Analytes | (% w/w) | (% w/w) | (% w/w) | (% w/w) |
| WS09420_Leaf | Withanolide | Withaferin A | 0.88 | 1.45 | 0.76 | 1.57 |
| (70% ethanol) | aglycones | Withastromonolide | | | 0.10 | |
| | | Withanolide A | | | 0.05 | |
| | | Withanolide B | | | 0.02 | |
| | | 27-Hydroxy Withanone | | | 0.08 | |
| | | Withanone | | | 0.00 | |
| | Withanolide | Withanoside IV | 0.57 | | 0.40 | |
| | glycosides | Withanoside V | | | 0.17 | |
| WS09320_Root | Withanolide | Withaferin A | 1.03 | 1.58 | 0.99 | 1.85 |
| (70% ethanol) | aglycones | Withastromonolide | | | 0.10 | |
| | | Withanolide A | | | 0.07 | |
| | | Withanolide B | | | 0.02 | |
| | | 27-Hydroxy Withanone | | | 0.07 | |
| | | Withanone | | | 0.00 | |
| | Withanolide | Withanoside IV | 0.55 | | 0.40 | |
| | glycosides | Withanoside V | | | 0.19 | |
| WS09220_Leaf:Root | Withanolide | Withaferin A | 0.71 | 1.21 | 0.56 | 1.45 |
| (70% ethanol) | aglycones | Withastromonolide | | | 0.12 | |
| | | Withanolide A | | | 0.08 | |
| | | Withanolide B | | | 0.02 | |
| | | 27-Hydroxy Withanone | | | 0.05 | |
| | | Withanone | | | 0.00 | |
| | Withanolide | Withanoside IV | 0.49 | | 0.43 | |
| | glycosides | Withanoside V | | | 0.19 | |
| WS08920_Leaf | Withanolide | Withaferin A | 0.94 | 1.45 | 1.36 | 2.11 |
| (100% ethanol) | aglycones | Withastromonolide | | | 0.07 | |
| Followed by water | | Withanolide A | | | 0.02 | |
| | | Withanolide B | | | 0.00 | |
| | | 27-Hydroxy Withanone | | | 0.12 | |
| | | Withanone | | | 0.00 | |
| | Withanolide | Withanoside IV | 0.51 | | 0.39 | |
| | glycosides | Withanoside V | | | 0.16 | |
| WS09020_Root | Withanolide | Withaferin A | 0.64 | 0.99 | 0.60 | 1.09 |
| (100% ethanol) | aglycones | Withastromonolide | | | 0.05 | |
| Followed by water | | Withanolide A | | | 0.05 | |
| | | Withanolide B | | | 0.01 | |
| | | 27-Hydroxy Withanone | | | 0.05 | |
| | | Withanone | | | 0.00 | |
| | Withanolide | Withanoside IV | 0.34 | | 0.23 | |
| | glycosides | Withanoside V | | | 0.10 | |
| WS09120_Leaf:Root | Withanolide | Withaferin A | 1.75 | 2.46 | 1.64 | 2.81 |
| (100% ethanol) | aglycones | Withastromonolide | | | 0.15 | |
| | | Withanolide A | | | 0.14 | |
| | | Withanolide B | | | 0.03 | |
| | | 27-Hydroxy Withanone | | | 0.10 | |
| | | Withanone | | | 0.00 | |
| | Withanolide | Withanoside IV | 0.70 | | 0.47 | |
| | glycosides | Withanoside V | | | 0.27 | |

TABLE 87
Assay Plate 1
Analysis

| | | Standard | | |
|---|---|---|---|---|
| | Average | Deviation | Assay fold | Z′ |
| 100% Proliferation | 21647 | 1706 | 2405 | 0.76 |
| (Cell Control) | | | | |
| 0% Proliferation | 9 | 1 | | |
| (Media only) | | | | |

TABLE 88
Plate 1 - Doxorubicin 10 uM
Doxorubicin @ 10 uM

% Inhibition

| Average | | | | | | Average |
|---|---|---|---|---|---|---|
| n1 | n2 | n3 | n1 | n2 | n3 | (SD) |
| 17 | 10 | 13 | 100 | 100 | 100 | 0.0 |

TABLE 89
Assay Plate 2
Analysis

| | | Standard | | |
|---|---|---|---|---|
| | Average | Deviation | Assay fold | Z′ |
| 100% Proliferation | 22538 | 2131 | 1439 | 0.72 |
| (Cell Control) | | | | |
| 0% Proliferation | 16 | 4 | | |
| (Media only) | | | | |

TABLE 90
Plate 2 - Doxorubicin 10 uM
Doxorubicin @ 10 uM

% Inhibition

| Average | | | | | | Average |
|---|---|---|---|---|---|---|
| n1 | n2 | n3 | n1 | n2 | n3 | (SD) |
| 10 | 11 | 10 | 100 | 100 | 100 | 0.0 |

TABLE 91
IC₅₀ of Different Examples of Withania somnifera (Sensoril ®-AWE)
Hydroethanolic Extracts

| | Withania somnifera | IC₅₀ | |
|---|---|---|---|
| Sample/Lot # | part used | (ug/ml) | Extraction Process |
| 1/#WS09120 | Nepal (Root + Leaf) | 17.5 | Ethanol followed by aqueous |
| 2/#WS08920 | Leaf | 19.2 | Ethanol followed by aqueous |
| 3/#WS09920 | Root | 28.1 | Ethanol followed by aqueous |
| 4/#WS09820 | Leaf + Root | 26.7 | Ethanol followed by aqueous |
| 5/#WS09020 | Leaf + Root | 48.7 | Ethanol followed by aqueous |
| 6/#WS10020 | Leaf + Root | 27.78 | Ethanol followed by aqueous |

TABLE 92
Withania somnifera (Sensoril ®-AWE) Hydroethanolic
Extract Sample 1 (“Nepal,” Root + Leaf)
Sensoril (Nepal)

| | Conc. | Raw data | | % Inhibition | | |
|---|---|---|---|---|---|---|
| Doses | (ug · ml | n1 | n2 | n1 | n2 | Avg |
| 1 | 400 | 29 | 24 | 100 | 100 | 100 |
| 2 | 200 | 19 | 18 | 100 | 100 | 100 |
| 3 | 100 | 35 | 29 | 100 | 100 | 100 |
| 4 | 50 | 100 | 92 | 100 | 100 | 100 |
| 5 | 25 | 1824 | 1972 | 92 | 91 | 91 |
| 6 | 13 | 21358 | 21524 | 1 | 1 | 1 |
| 7 | 6 | 26242 | 25684 | −21 | −19 | −20 |
| 8 | 3 | 23562 | 21011 | −9 | 3 | −3 |

TABLE 93
Withania somnifera (Sensoril ®-AWE) Hydroethanolic Extract Sample 2
Sensoril Lot # WS08920

| | | Raw data | | % Inhibition | | |
|---|---|---|---|---|---|---|
| Doses | Conc. | n1 | n2 | n1 | n2 | Avg |
| 1 | 400 | 30 | 31 | 100 | 100 | 100 |
| 2 | 200 | 18 | 17 | 100 | 100 | 100 |
| 3 | 100 | 69 | 64 | 100 | 100 | 100 |
| 4 | 50 | 272 | 251 | 99 | 99 | 99 |
| 5 | 25 | 6670 | 5389 | 69 | 75 | 72 |
| 6 | 13 | 23535 | 24001 | −9 | −11 | −10 |
| 7 | 6 | 28535 | 27853 | −32 | −29 | −30 |
| 8 | 3 | 26615 | 27234 | −23 | −26 | −24 |

TABLE 94
Withania somnifera (Sensoril ®-AWE) Hydroethanolic Extract Sample 3
Sensoril # WS 9920

| | | Raw data | | % Inhibition | | |
|---|---|---|---|---|---|---|
| Doses | Conc. | n1 | n2 | n1 | n2 | Avg |
| 1 | 400 | 12 | 9 | 100 | 100 | 100 |
| 2 | 200 | 23 | 25 | 100 | 100 | 100 |
| 3 | 100 | 84 | 66 | 100 | 100 | 100 |
| 4 | 50 | 806 | 636 | 96 | 97 | 97 |
| 5 | 25 | 19036 | 16768 | 12 | 23 | 17 |
| 6 | 13 | 27425 | 24977 | −27 | −15 | −21 |
| 7 | 6 | 26766 | 26654 | −24 | −23 | −23 |
| 8 | 3 | 27133 | 26806 | −25 | −24 | −25 |

TABLE 95
Withania somnifera (Sensoril ®-AWE) Hydroethanolic Extract Sample 4
Sensoril # WS 9820

| | Conc. | Raw Data | | % Inhibition | | |
|---|---|---|---|---|---|---|
| Doses | (ug, ml | n1 | n2 | n1 | n2 | Avg |
| 1 | 400 | 23 | 21 | 100 | 100 | 100 |
| 2 | 200 | 74 | 30 | 100 | 100 | 100 |
| 3 | 100 | 112 | 87 | 100 | 100 | 100 |
| 4 | 50 | 745 | 620 | 97 | 97 | 97 |
| 5 | 25 | 15188 | 15170 | 30 | 30 | 30 |
| 6 | 13 | 24973 | 24967 | −15 | −15 | −15 |
| 7 | 6 | 26116 | 24970 | −21 | −15 | −18 |
| 8 | 3 | 25775 | 25723 | −19 | −19 | −19 |

TABLE 96
Withania somnifera (Sensoril ®-AWE) Hydroethanolic Extract Sample 5
Sensoril # WS 9020

| | | Raw data | | % Inhibition | | |
|---|---|---|---|---|---|---|
| Doses | Conc. | n1 | n2 | n1 | n2 | Avg |
| 1 | 400 | 44 | 85 | 100 | 100 | 100 |
| 2 | 200 | 88 | 68 | 100 | 100 | 100 |
| 3 | 100 | 393 | 357 | 98 | 98 | 98 |
| 4 | 50 | 10464 | 9713 | 52 | 55 | 53 |
| 5 | 25 | 21450 | 20206 | 1 | 7 | 4 |
| 6 | 13 | 24551 | 22741 | −13 | −5 | −9 |
| 7 | 6 | 21259 | 19835 | 2 | 8 | 5 |
| 8 | 3 | 21984 | 19733 | −2 | 9 | 4 |

TABLE 97
Withania somnifera (Sensoril ®-AWE) Hydroethanolic Extract Sample 6
Sensoril # WS 10020

| | | Raw data | | % Inhibition | | |
|---|---|---|---|---|---|---|
| Doses | Conc. | n1 | n2 | n1 | n2 | Avg |
| 1 | 400 | 29 | 27 | 100 | 100 | 100 |
| 2 | 200 | 25 | 22 | 100 | 100 | 100 |
| 3 | 100 | 78 | 66 | 100 | 100 | 100 |
| 4 | 50 | 483 | 356 | 98 | 98 | 98 |
| 5 | 25 | 15121 | 12884 | 30 | 40 | 35 |
| 6 | 13 | 23885 | 21962 | −10 | −1 | −6 |
| 7 | 6 | 21871 | 21417 | −1 | 1 | 0 |
| 8 | 3 | 21346 | 20448 | 1 | 6 | 3 |

### Example X

### Inhibition of Histiocytic Lymphoma and Pancreatic Cancer Cells with Different Extracts

Histiocytic lymphoma (U-937) cells and Pancreatic cancer (Panc-1) cells were incubated for 72 hours with 100 ug/ml, 30 ug/ml, and 10 ug/ml each of fresh samples of AyuFlex®, Capros®, Phyto-BGS®, 2 different samples of Shilajit (PrimaVie®) extracts, several different samples of hydroethanolic Withania somnifera (Sensoril®-AWE) extracts, and Ayuric®. Cells were plated at 1.25 k/well in 384 well plates. Standard of care drug doxorubicin was also tested at a 10 uM concentration. Anti-proliferative assays were generally performed as described in Example I above, in triplicate, with detection reagent CellTiterGlo. Tables 98-99 show data for controls for assays on histiocytic lymphoma (U-937) cells, and that the data passed quality control parameters. Tables 101-102 show data for controls for assays on pancreatic cancer (Panc-1) cells, and that the data passed quality control parameters.

Tables 100 and 103 show data relating to the inhibition of histiocytic lymphoma (U-937) cells and pancreatic cancer (Panc-1) cells by extracts of this invention. The assay is generally as described in Example I.

TABLE 98
Assay Plate
Analysis

| | | Standard | | |
|---|---|---|---|---|
| | Average | Deviation | Assay fold | Z′ |
| 100% Proliferation | 52629 | 4256 | 1847 | 0.76 |
| (Cell Control) | | | | |
| 0% Proliferation | 29 | 4 | | |
| (Media only) | | | | |

TABLE 99
Doxorubicin 10 uM
Doxorubicin @ 10 uM

% Inhibition

| Average | | | | | | Average |
|---|---|---|---|---|---|---|
| n1 | n2 | n3 | n1 | n2 | n3 | (SD) |
| 58 | 50 | 70 | 100 | 100 | 100 | 100(0) |

TABLE 100
Inhibition of histiocytic lymphoma cancer cell (U-937 cell line) proliferation with different extracts

| | | | | | | | | | | | 100 ug/ml | | 30 ug/ml | | 10 ug/ml | |
|---|---|---|---|---|---|---|---|---|---|---|---|---|---|---|---|---|
| S. | | % Inhibition | | | | | | | | | Avg % | | Avg % | | Avg % | |
| No. | Extracts | 100 ug/ml | | | 30 ug/ml | | | 10 ug/ml | | | Inhibition | SD | Inhibition | SD | Inhibition | SD |
| 1 | Ayuflex | 61 | 58 | 64 | 12 | 7 | 10 | −9 | −11 | 0 | 61 | 3 | 9 | 3 | −6 | 6 |
| 2 | Capros | −6 | −7 | −8 | −12 | −6 | 2 | −6 | −6 | −3 | −7 | 1 | −6 | 7 | −5 | 2 |
| 3 | Phyto-BGS | −19 | −19 | −17 | −11 | −9 | −3 | −9 | −8 | −3 | −18 | 1 | −8 | 4 | −7 | 3 |
| 4 | Primavie | −3 | −13 | −1 | −13 | −13 | −15 | −7 | −12 | −18 | −5 | 6 | −13 | 1 | −12 | 6 |
| 5 | Sensoril | −19 | −5 | 0 | 4 | 10 | 22 | 11 | 9 | −3 | −8 | 10 | 12 | 9 | 6 | 8 |
| 6 | Ayuric | 44 | 45 | 45 | −4 | 1 | 13 | −8 | −11 | −12 | 45 | 0 | 4 | 9 | −10 | 2 |
| 7 | Sensoril | 99 | 99 | 99 | 97 | 97 | 98 | 42 | 40 | 38 | 99 | 0 | 97 | 0 | 40 | 2 |
| | (Nepal) | | | | | | | | | | | | | | | |
| 8 | Sensoril | 6 | 3 | 8 | 21 | 20 | 20 | 20 | 21 | 21 | 6 | 2 | 20 | 0 | 21 | 0 |
| | (Patel) | | | | | | | | | | | | | | | |
| 9 | Sensoril | 9 | 6 | 8 | −4 | 4 | 8 | 1 | −8 | −1 | 7 | 1 | 2 | 6 | −3 | 5 |
| | (Fitochem) | | | | | | | | | | | | | | | |
| 10 | Primavie | 30 | 29 | 36 | 16 | 18 | 15 | NA | NA | NA | 31 | 4 | 16 | 2 | NA | NA |
| | (Fitochem) | | | | | | | | | | | | | | | |

TABLE 101
Assay Plate
Analysis

| | | Standard | | |
|---|---|---|---|---|
| | Average | Deviation | Assay fold | Z′ |
| 100% Proliferation | 63086 | 3225 | 1043 | 0.85 |
| (Cell Control) | | | | |
| 0% Proliferation | 61 | 9 | | |
| (Media only) | | | | |

TABLE 102
Doxorubicin 10 uM
Doxorubicin @ 10 uM

% Inhibition

| Average | | | | | | Average |
|---|---|---|---|---|---|---|
| n1 | n2 | n3 | n1 | n2 | n3 | (SD) |
| 3908 | 4011 | 3437 | 94 | 94 | 95 | 94(0) |

TABLE 103
Inhibition of pancreatic cancer cell (Panc-1 cell line) proliferation with different extracts

| | | | | | | | | | | | 100 ug/ml | | 30 ug/ml | | 10 ug/ml | |
|---|---|---|---|---|---|---|---|---|---|---|---|---|---|---|---|---|
| S. | | % Inhibition | | | | | | | | | Avg % | | Avg % | | Avg % | |
| No. | Extracts | 100 ug/ml | | | 30 ug/ml | | | 10 ug/ml | | | Inhibition | SD | Inhibition | SD | Inhibition | SD |
| 1 | Ayuflex | 36 | 36 | 35 | 2 | −1 | −1 | −4 | 0 | 4 | 36 | 1 | 0 | 2 | 0 | 4 |
| 2 | Capros | 15 | 9 | 12 | 0 | 4 | 1 | 10 | 12 | 11 | 12 | 3 | 2 | 2 | 11 | 1 |
| 3 | Phyto-BGS | 6 | 5 | 0 | 3 | 4 | 4 | 5 | 5 | 4 | 4 | 3 | 4 | 1 | 5 | 1 |
| 4 | Primavie | −1 | 3 | 5 | 6 | 7 | 4 | 1 | 8 | 2 | 2 | 3 | 6 | 1 | 4 | 4 |
| 5 | Sensoril | 13 | 8 | 9 | 9 | 9 | 5 | 8 | 18 | 15 | 10 | 3 | 8 | 2 | 14 | 5 |
| | (Sakti) | | | | | | | | | | | | | | | |
| 6 | Ayuric | 32 | 29 | 35 | 12 | 14 | 13 | 18 | 18 | 22 | 32 | 3 | 13 | 1 | 19 | 2 |
| 7 | Sensoril | 55 | 56 | 55 | 71 | 67 | 60 | 24 | 30 | 25 | 55 | 1 | 66 | 6 | 26 | 3 |
| | (Nepal) | | | | | | | | | | | | | | | |

| | | | | | | | | | | | | | | |
|---|---|---|---|---|---|---|---|---|---|---|---|---|---|---|
| 8 | Sensoril | 22 | 22 | 28 | Not Done | 27 | 23 | 25 | 24 | 4 | NA | NA | 25 | 2 |
| | Patel | | | | | | | | | | | | | |

| | | | | | | | | | | | | | | |
|---|---|---|---|---|---|---|---|---|---|---|---|---|---|---|
| 9 | Sensoril | 17 | 16 | 21 | 20 | 18 | 14 | Not Done | 18 | 3 | 18 | 3 | NA | NA |
| | (Fitochem) | | | | | | | | | | | | | |

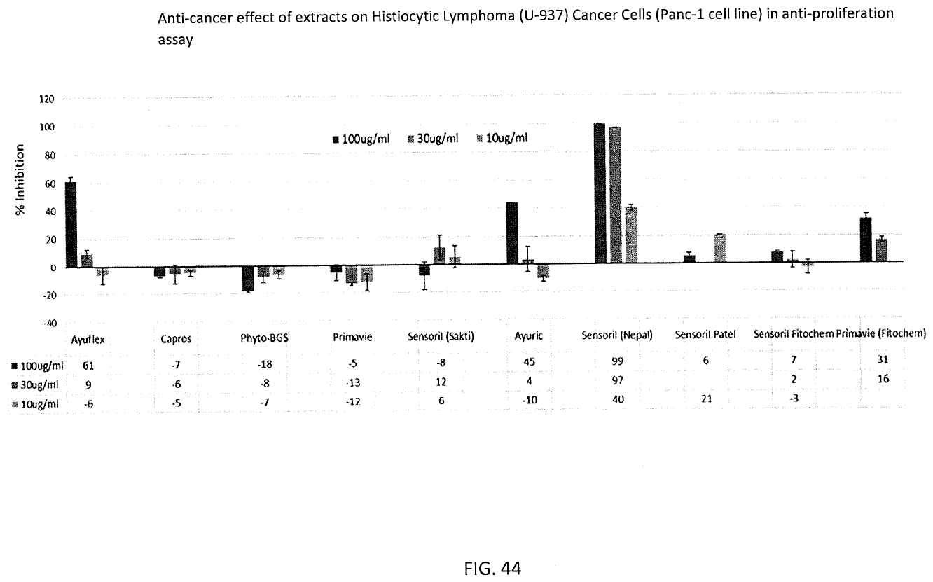

FIG. 44 shows the anti-cancer, anti-proliferative effects of extract compositions of this invention on histiocytic lymphoma (U-937 cell line) cancer cells. Hydroethanolic extracts of Withania somnifera (Sensoril®-AWE) showed the most potent anti-cancer effects against the histiocytic lymphoma cells, with about 100% inhibition at 100 ug/ml and 30 ug/ml doses, and 40% inhibition at the 10 ug/ml dose. The next most effective extracts were Ayuflex® and Ayuric®, which showed 60% and 45% inhibition at the 100 ug/ml doses. The assay control, doxorubicin, showed 100% inhibition at 10 uM.

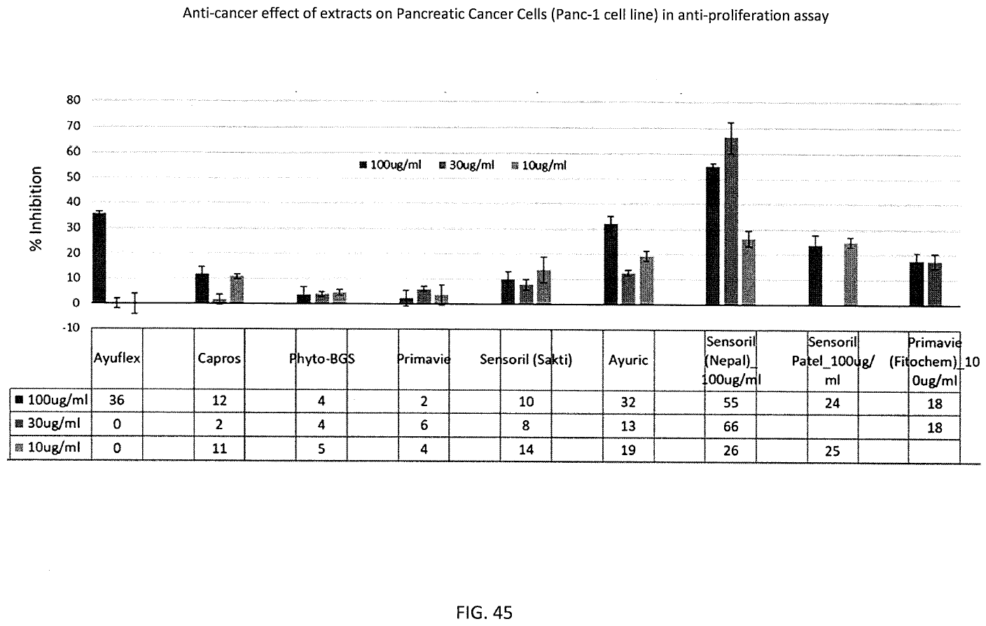

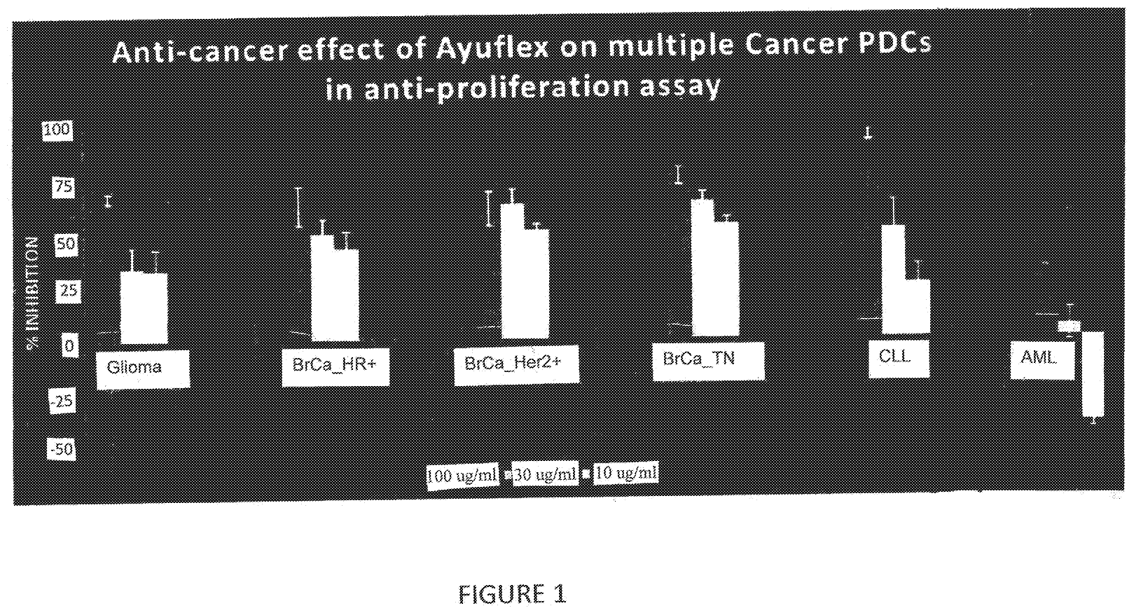

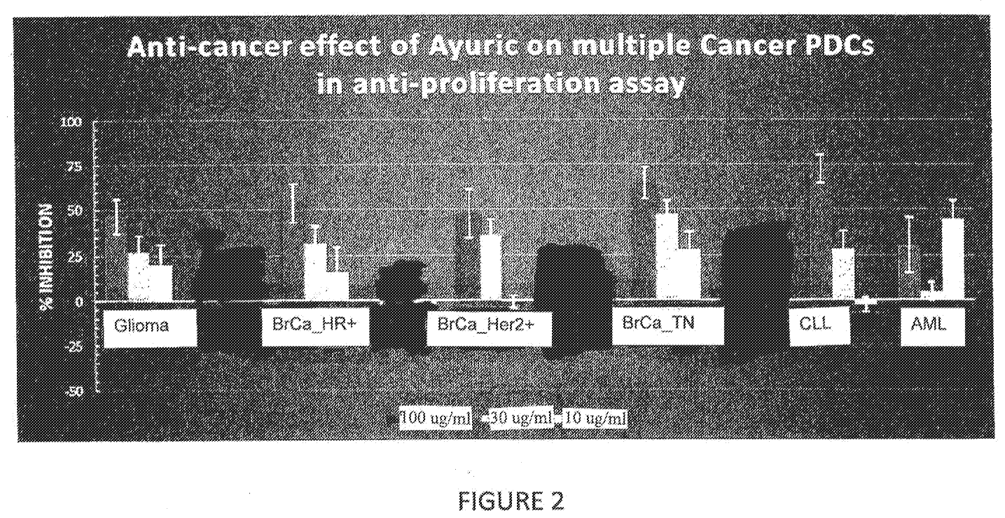

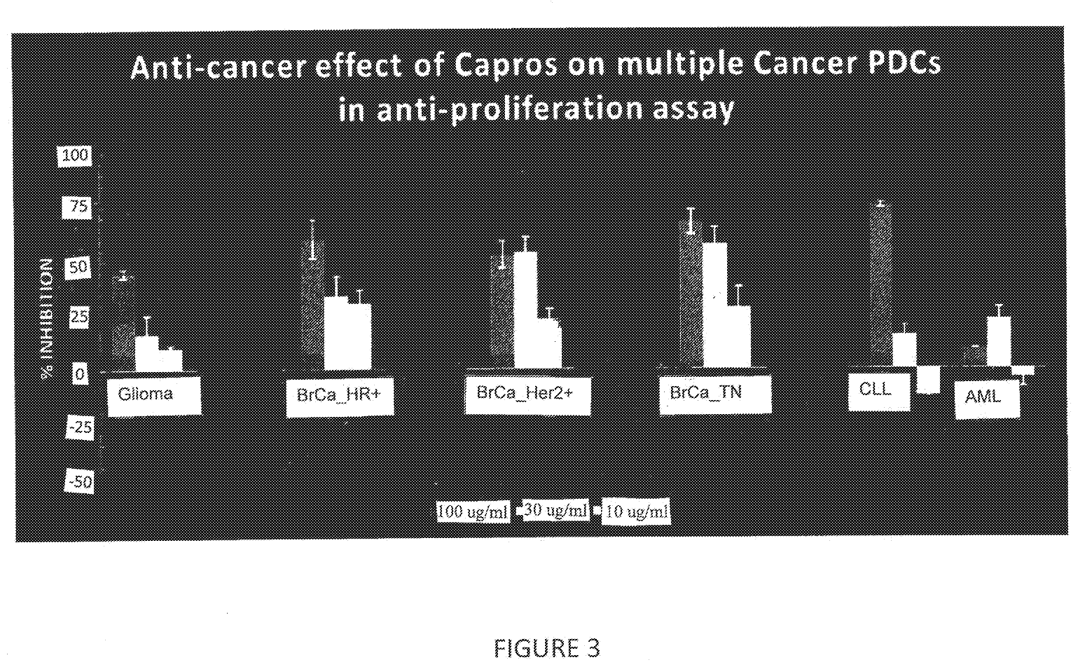

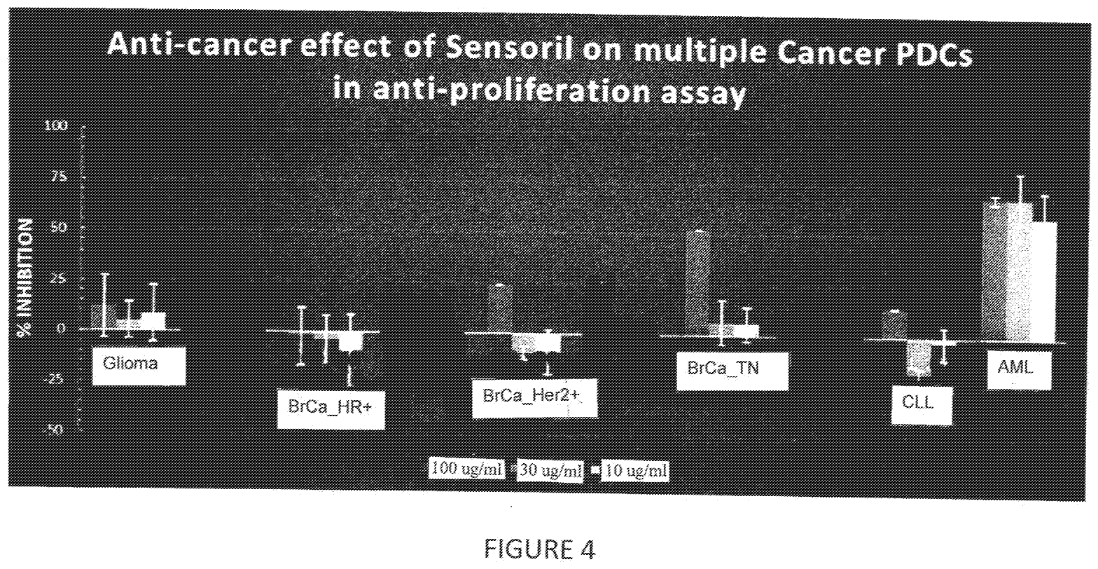

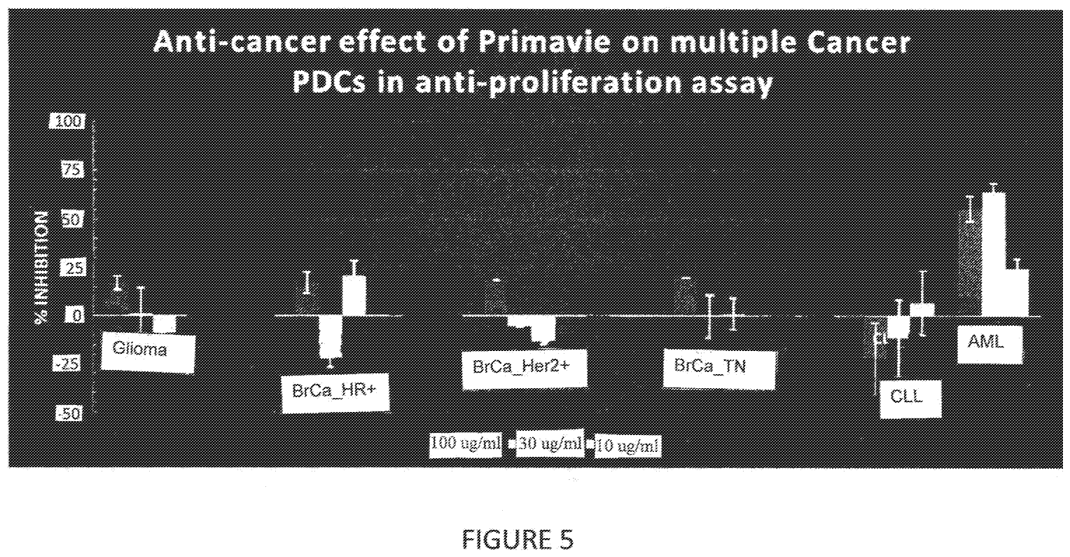

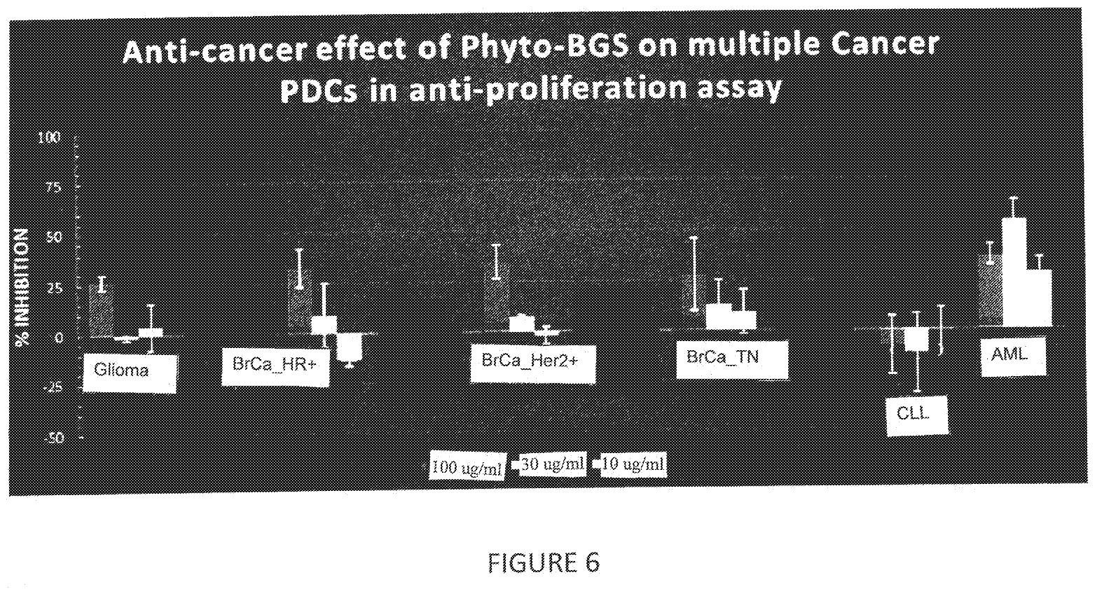

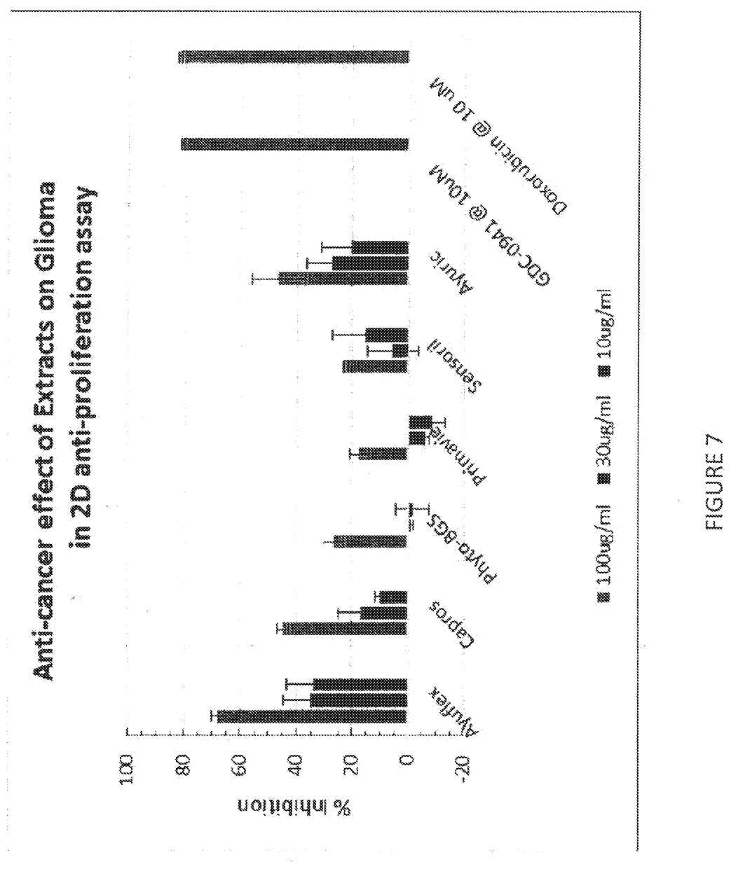

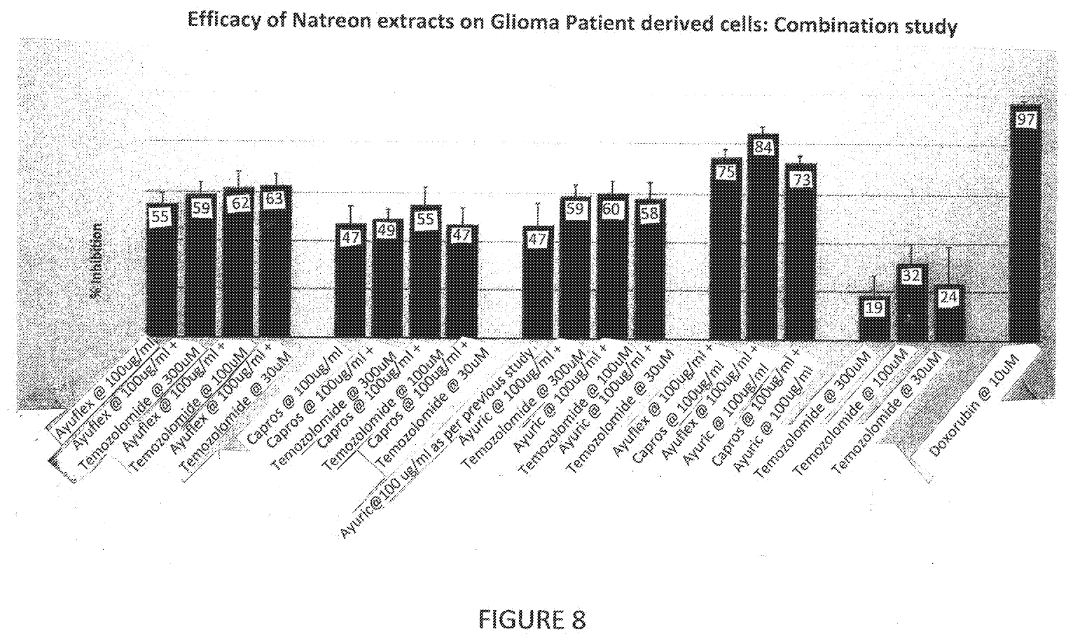

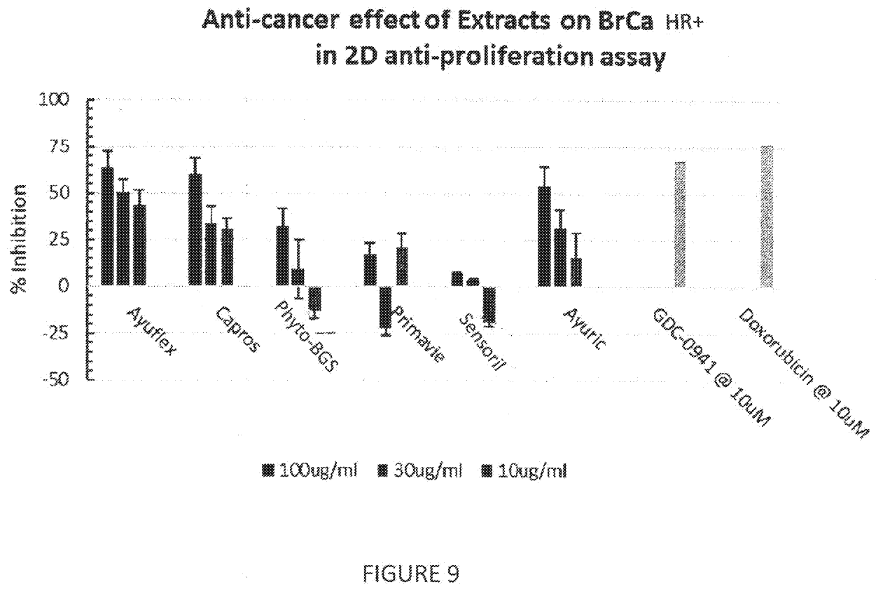

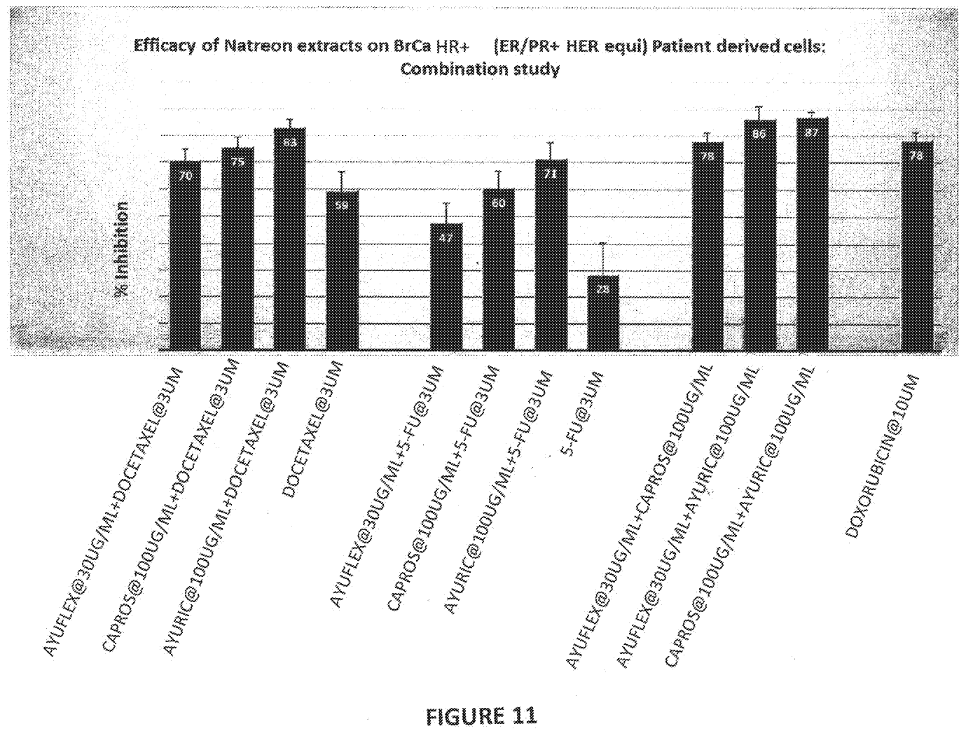

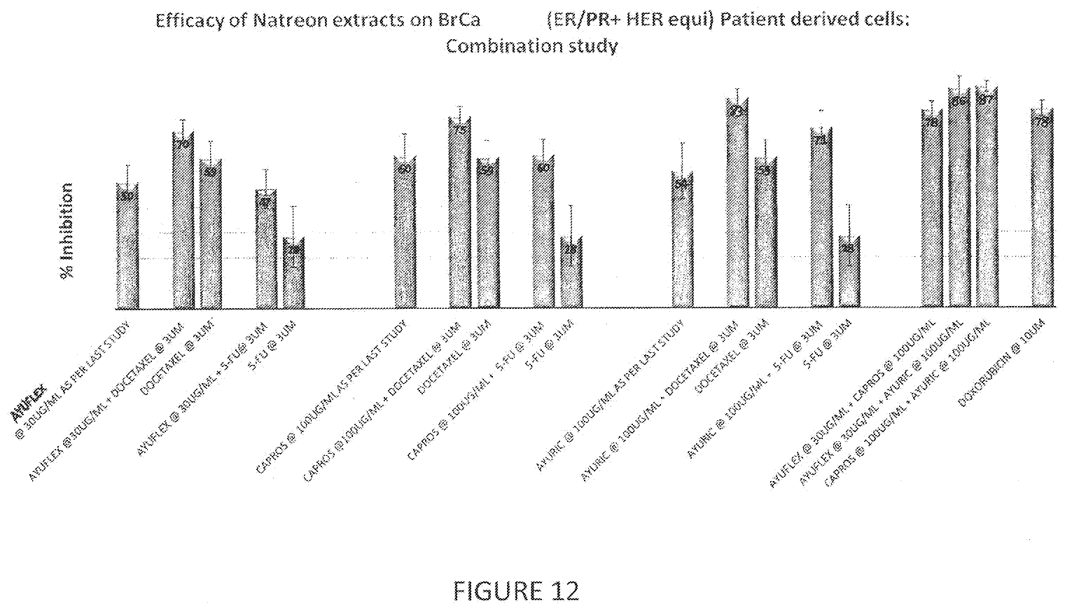

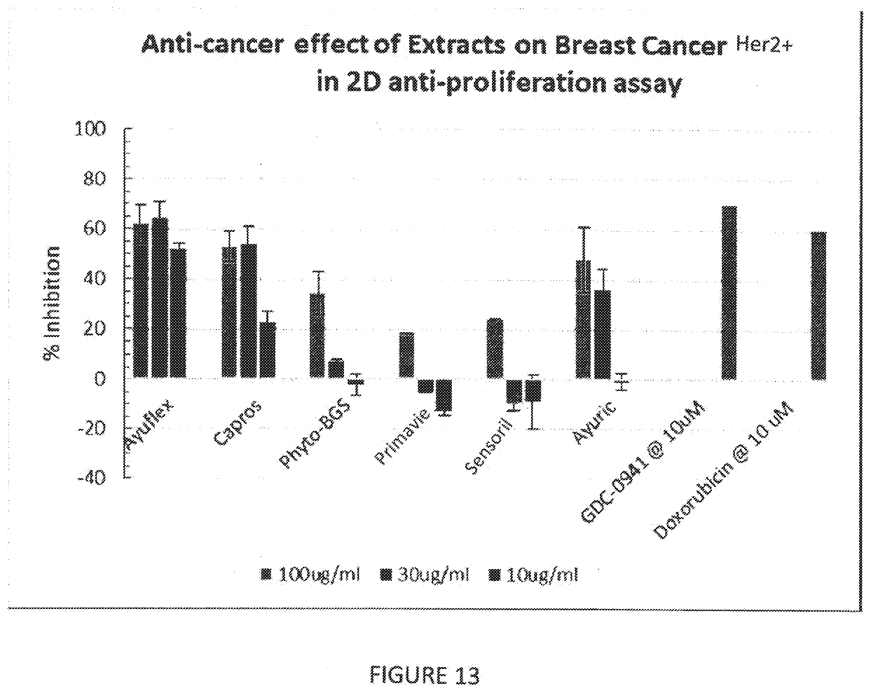

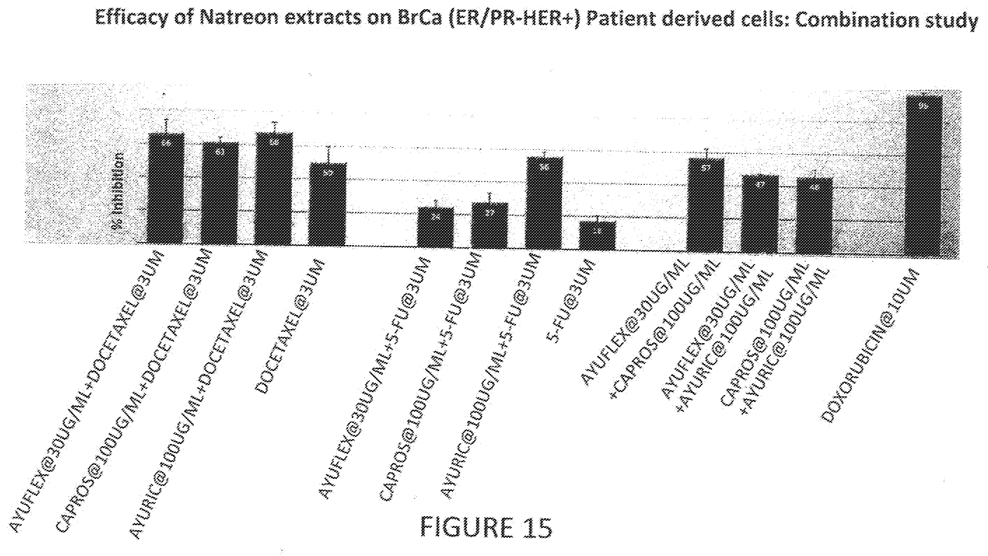

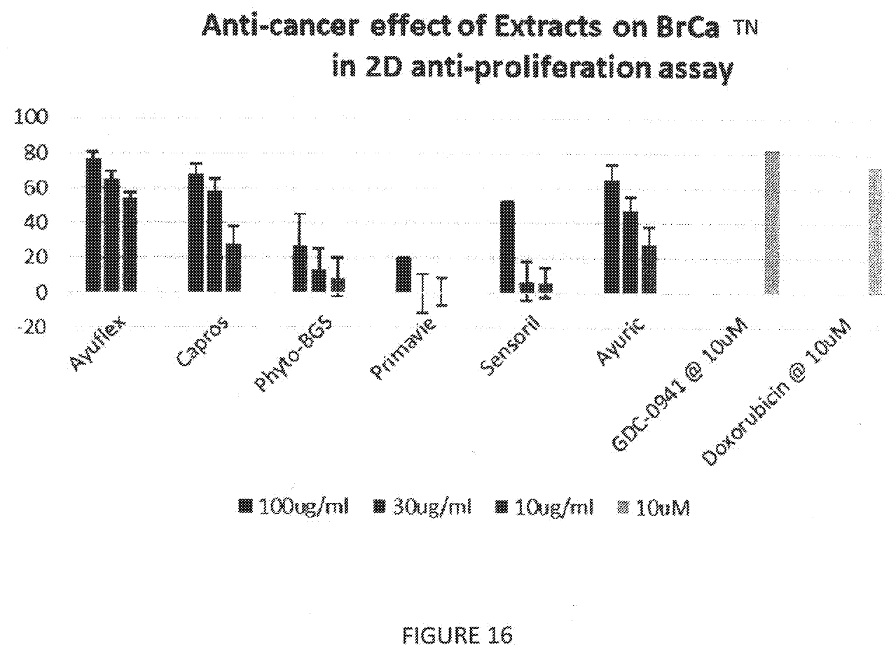

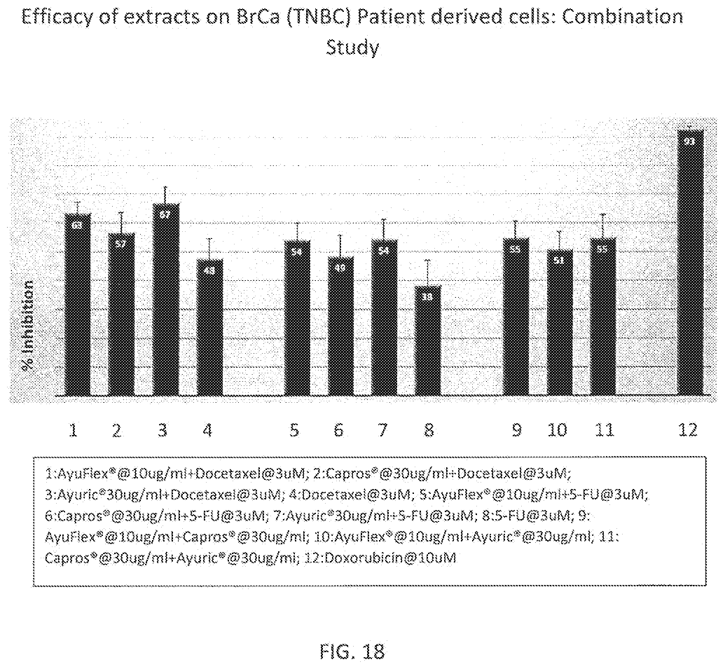

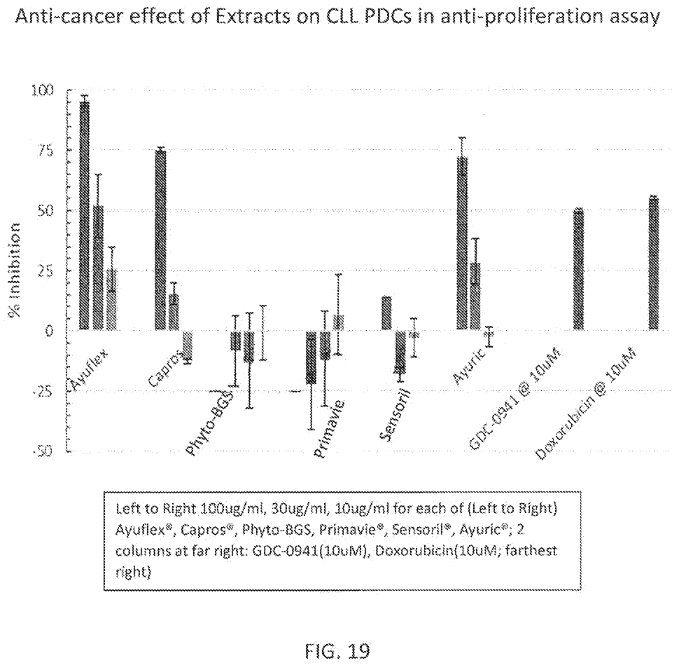

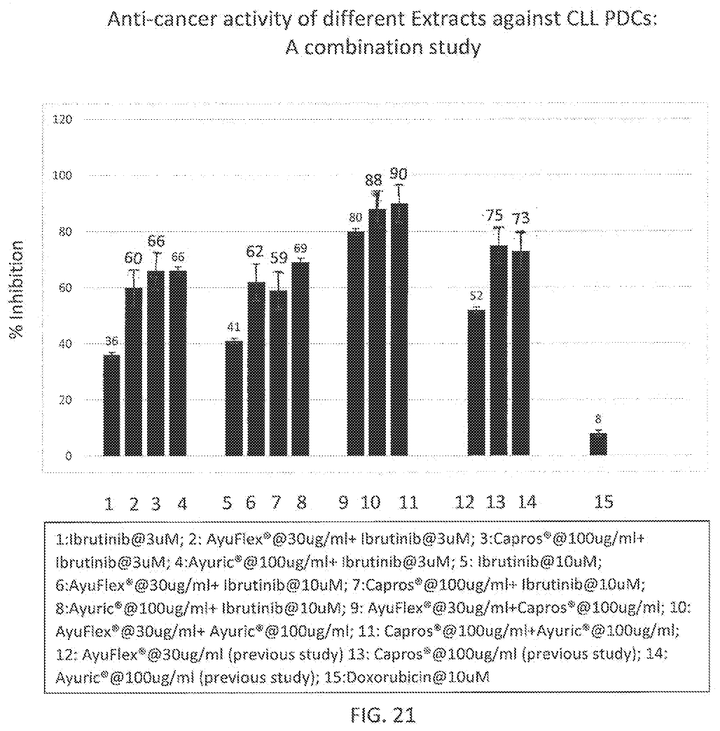

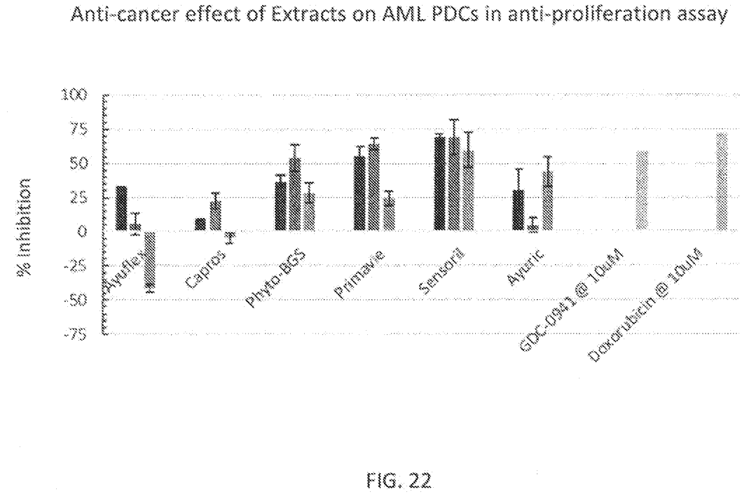

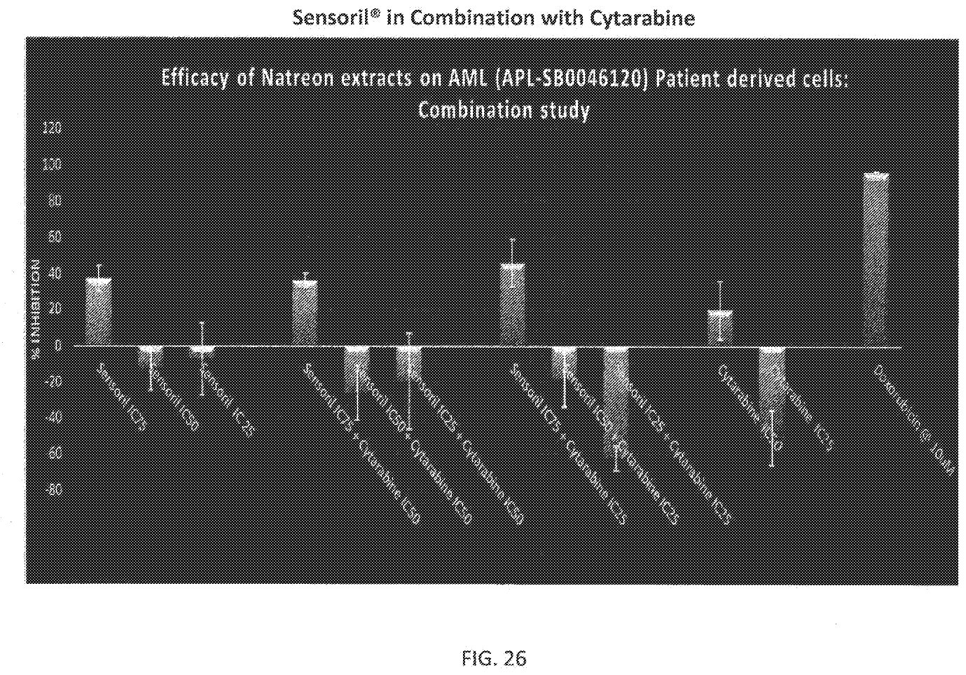

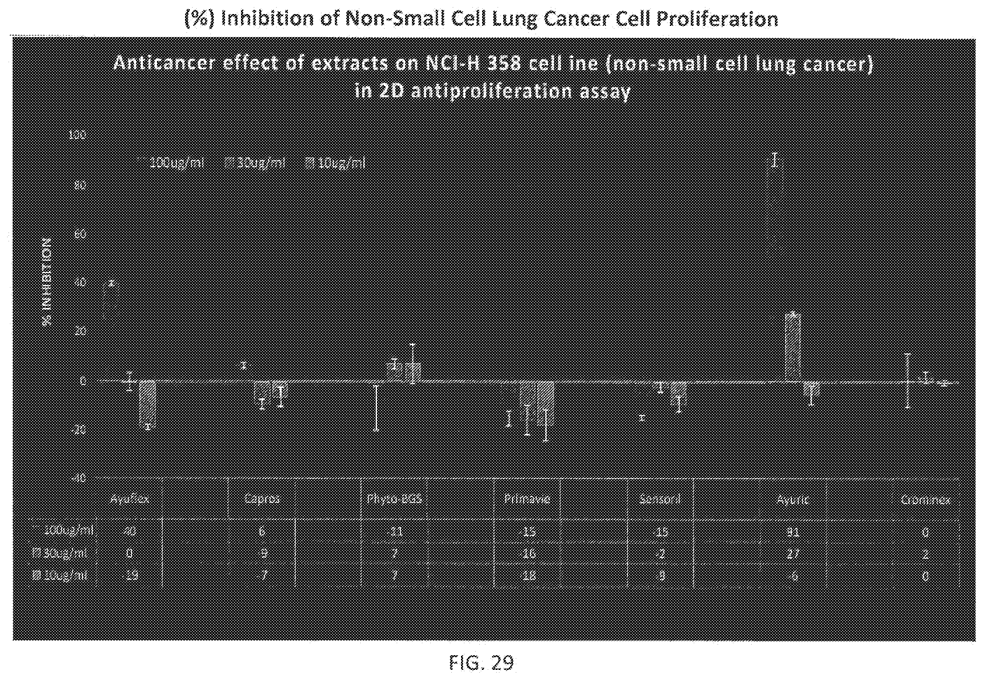

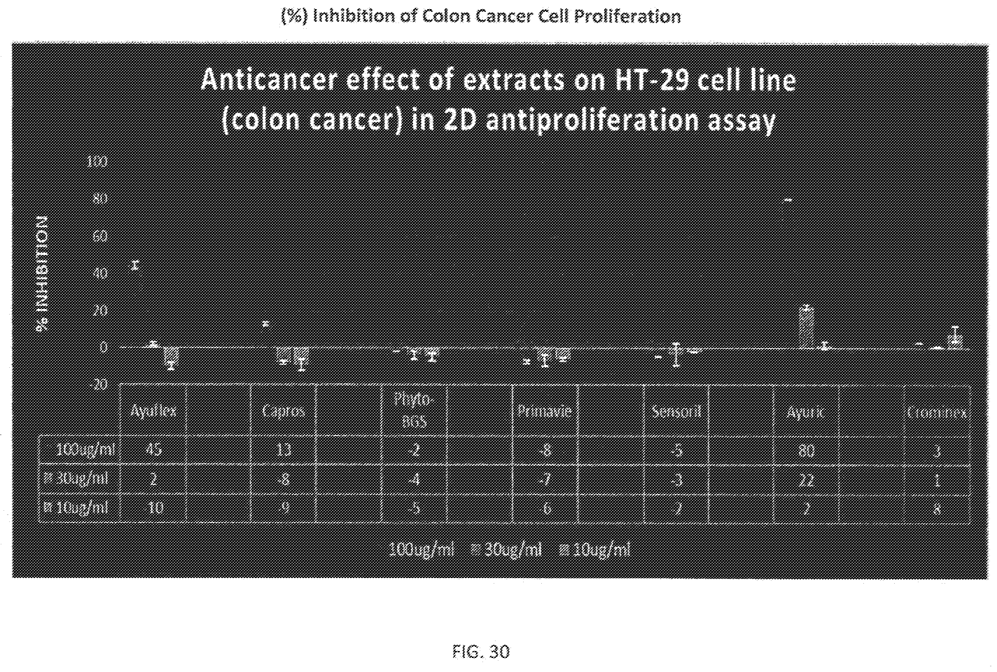

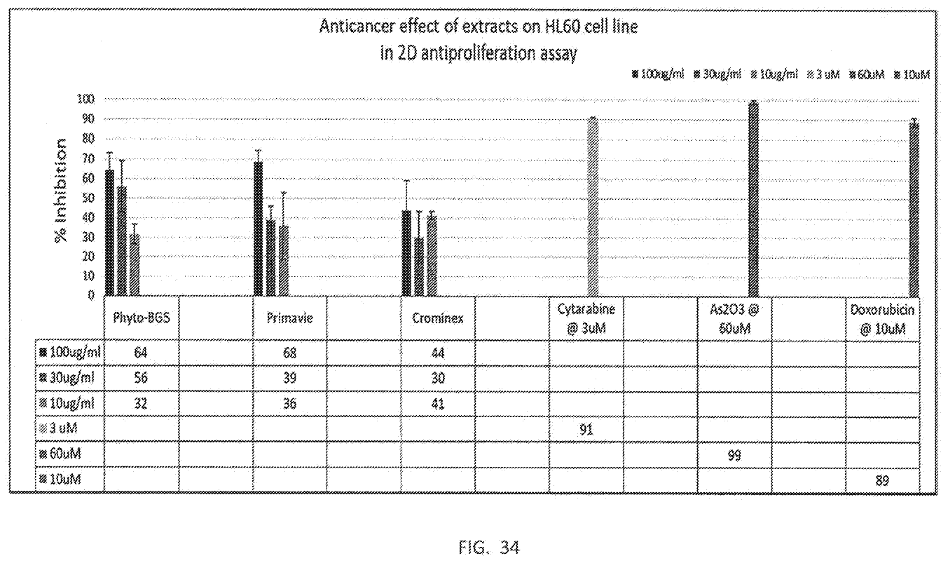

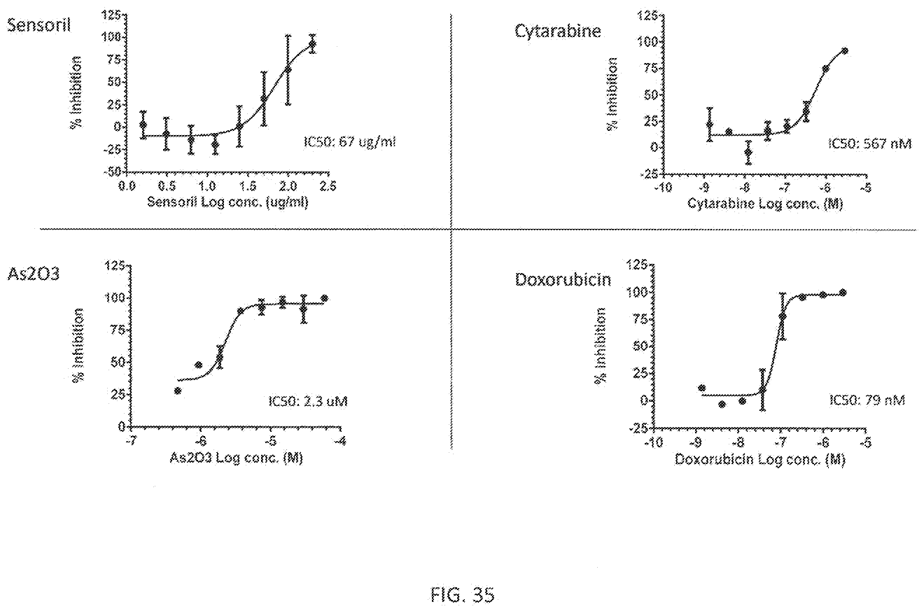

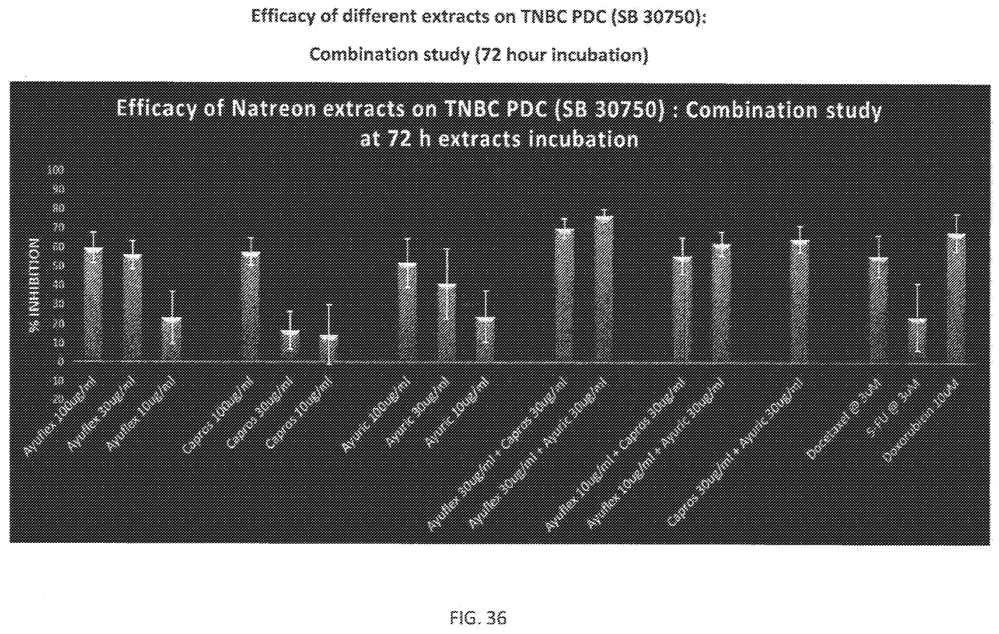

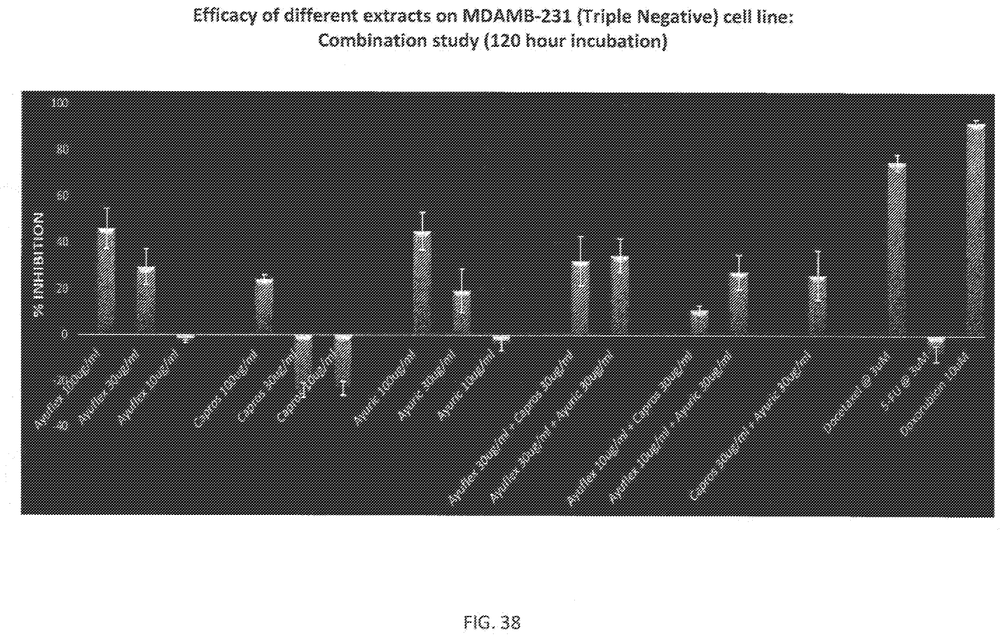

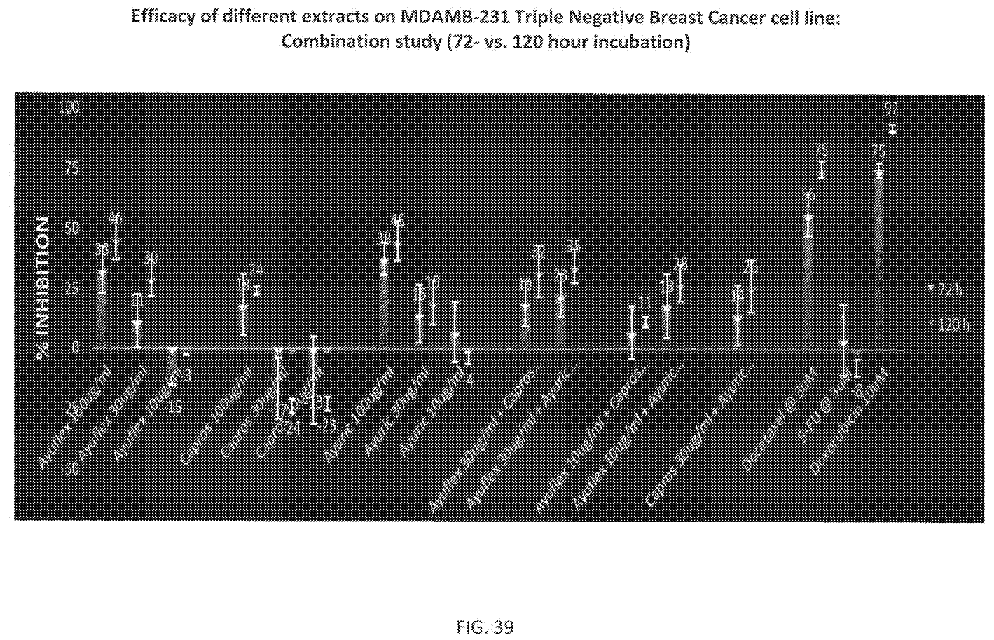

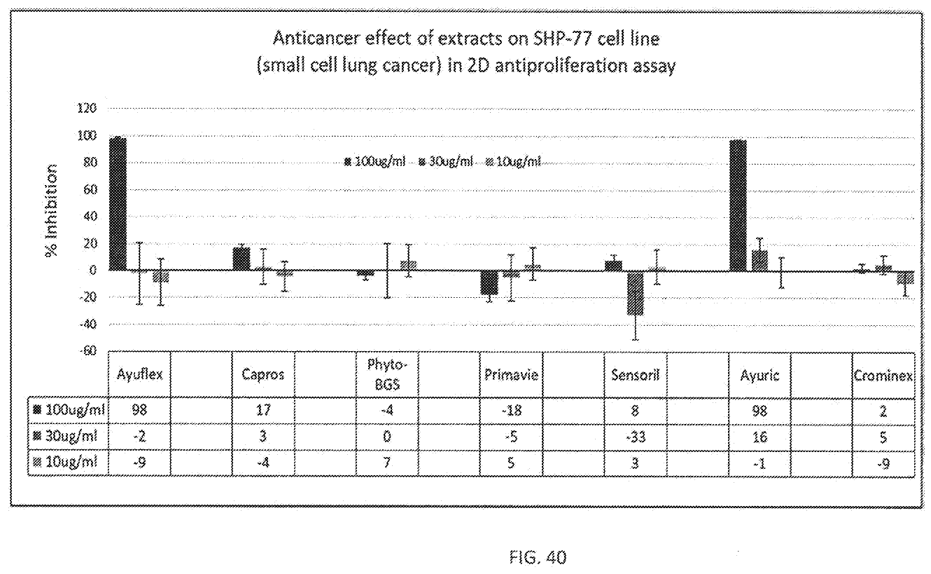

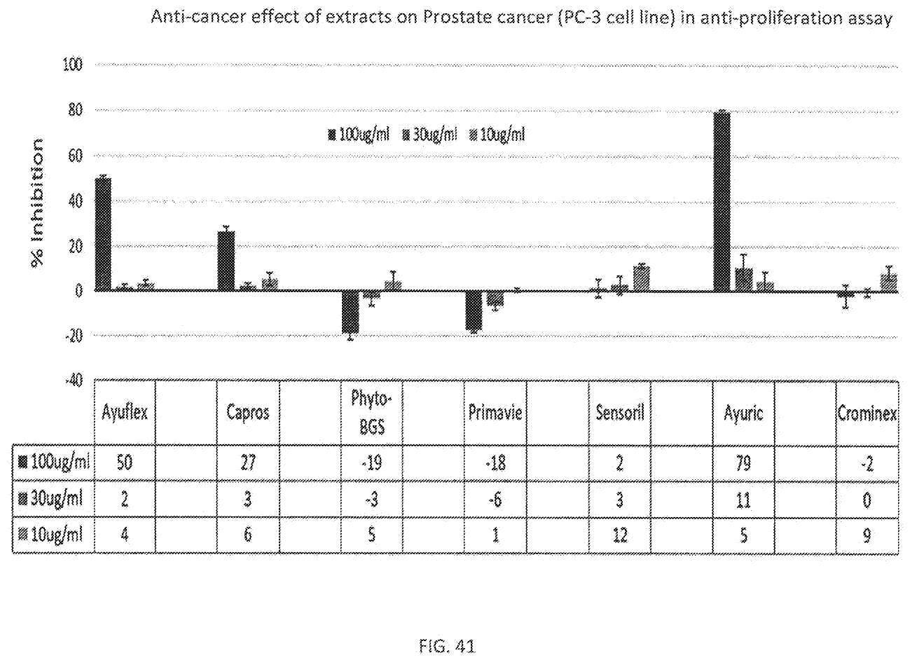

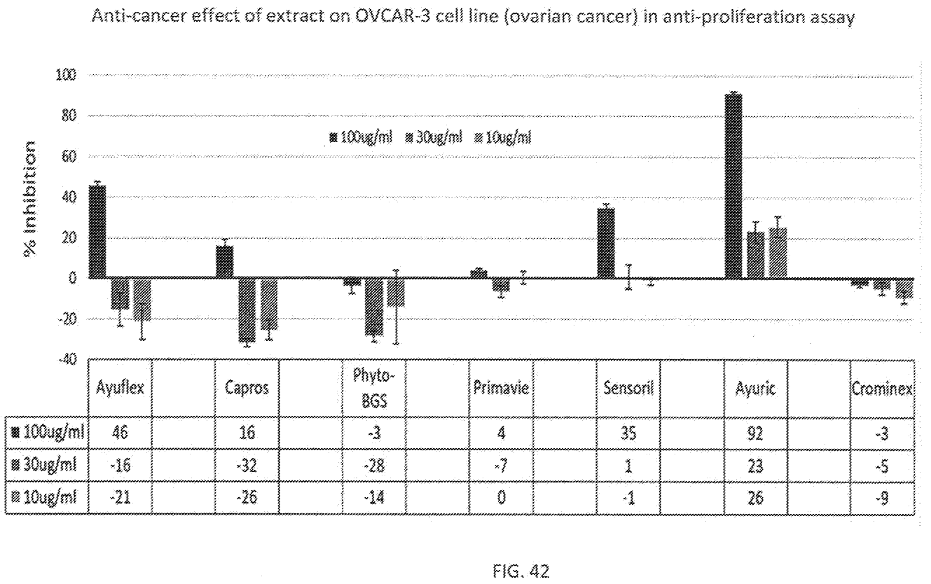

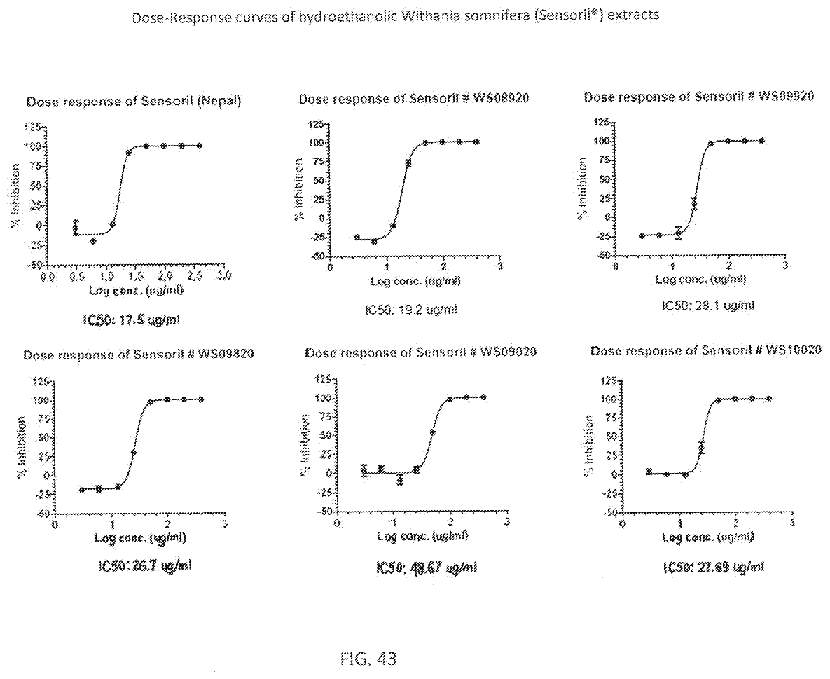

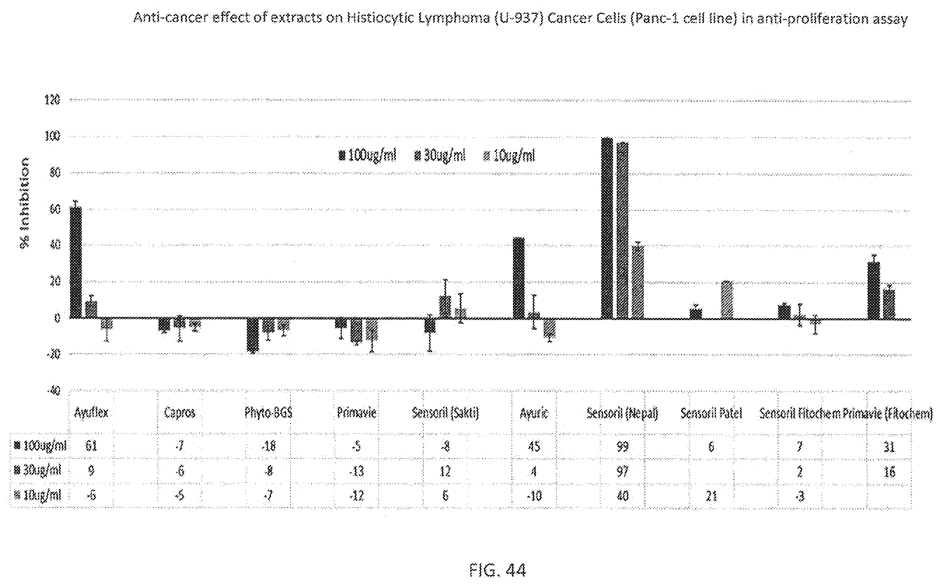

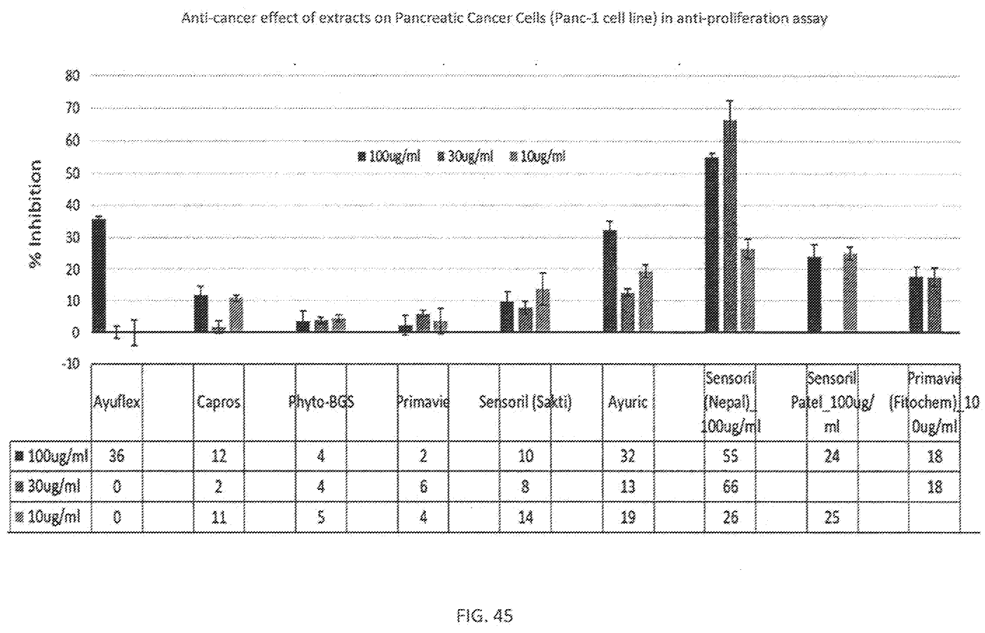

FIG. 45 shows the anti-cancer, anti-proliferative effects of extract compositions of this invention on pancreatic cancer (Panc-1 cell line) cancer cells. Hydroethanolic extracts of Withania somnifera (Sensoril®-AWE) showed the most potent anti-cancer effect, with 50-60% at 100 ug/ml and 30 ug/ml doses, followed by 26% inhibition at the 10 ug/ml concentration. The next most effective extract was Ayuflex®, showing greater than 30% inhibition of pancreatic cancer cell growth (Panc-1 cell line) at 100 ug/ml. The assay control, doxorubicin, showed 94% inhibition at 10 uM.

### Discussion

Compositions of the present invention provide anti-cancer activity, as shown by the remarkable inhibition of cancer cell proliferation by compositions of this invention in the above Examples. Anti-proliferative activity was seen with the present compositions in glioma cancer cells, breast cancer cells, chronic lymphocytic leukemia cancer cells, acute myeloid leukemia cancer cells, small cell lung cancer cells, non-small lung cancer cells, colon cancer cells, prostate cancer cells, pancreatic cancer cells, and ovarian cancer cells. Inhibition was seen with different extracts on difference cancer cell types, sometimes with similar or even synergistic effects with combinations of extracts and/or extract(s) with a known anti-cancer drug. See for instance FIGS. 8, 11, 15, 18, 27-28, 31-33, and 36.

For instance, Ayuflex® and Ayuric® inhibited the proliferation of several cancer cell types, including pancreatic, ovarian, prostatic, small cell lung cancer, and colon cancer cells. Ayuflex® and Ayuric® may be administered for instance with standard non-small cell lung cancer and colon cancer drugs, and other drugs useful in treating cancer, including a low dose of said drugs to achieve optimal efficacy and lower side effects of the drugs. In an embodiment, a composition of the present invention may be used with a standard of care cancer drug to minimize the amount of standard of care drug given to a subject. Given the harsh adverse and toxic effects of many known cancer drugs, the present invention provides a useful method for co-administration with such drugs.

Also for instance Sensoril® showed potent anti-cancer and anti-proliferative activity against different subtypes of AML, alone and synergistically in combination with drugs relevant to AML (Acute Myeloid Leukemia) and subtype APL (Acute Promyelocytic Leukemia (APL, APML)) treatment such as arsenic trioxide and doxorubicin. Hydroethanolic Sensoril®-AWE compositions were shown to be effective against AML HL60 cell line cancer cells. Sensoril® or Hydroethanolic Sensoril® (Sensoril®-AWE) may be administered with standard AML drugs, including at a low dose of the AML drugs, to achieve optimal efficacy and lower side effects of the drugs.

Several embodiments of the present invention are set out herein. In addition to the below, embodiments supported by the above Examples are intended as general embodiments of the invention, similar to the below.

A method of inhibiting the proliferation of cancer cells and/or cancer-associated cells comprising the steps of providing a composition comprising an extract of Terminalia chebula fruits, and applying said composition to said cancer cells to inhibit proliferation of the cells. In an embodiment, said composition is a standardized aqueous extract of the Terminalia chebula fruits, and said extract is Ayuflex®. In an embodiment, said cancer cells are glioma cells, breast cancer cells, chronic lymphocytic leukemia cells, acute myeloid leukemia cells, non-small cell lung cancer cells, small cell lung cancer cells, prostate cancer cells, ovarian cancer cells, pancreatic cancer cells, and/or colon cancer cells. In an embodiment, said cancer cells are chronic lymphocytic leukemia cells, glioma cells, prostate cancer cells, ovarian cancer cells, pancreatic cancer cells, breast cancer “HR+” cells, breast cancer “Her2+” cells, or breast cancer “triple negative” cells. A method of treating cancer in a subject in need thereof, and/or enhancing the treatment of cancer in a subject in need thereof, comprising the steps of providing a composition comprising an extract of Terminalia chebula fruits, and administering an effective amount of said composition to treat and/or enhance treatment of cancer in the subject. In an embodiment, said composition is a standardized aqueous extract of the Terminalia chebula fruits, and said extract is Ayuflex®. In an embodiment, said cancer is glioma, breast cancer, chronic lymphocytic leukemia, acute myeloid leukemia, non-small cell lung cancer, prostate cancer cells, ovarian cancer cells, pancreatic cancer cells, and/or colon cancer. In an embodiment, said cancer is chronic lymphocytic leukemia, glioma, prostate cancer cells, ovarian cancer cells, pancreatic cancer cells, breast cancer “HR+”, breast cancer “Her2+”, or breast cancer “triple negative”.

A method of inhibiting the proliferation of cancer cells comprising the steps of providing a composition comprising an extract of Terminalia bellerica fruits, and applying said composition to said cancer cells to inhibit proliferation of the cells. In an embodiment, said composition is a standardized aqueous extract of the Terminalia bellerica fruits, and said extract is Ayuric®. In an embodiment, said cancer cells are glioma cells, breast cancer cells, chronic lymphocytic leukemia cells, acute myeloid leukemia cells, non-small cell lung cancer cells, prostate cancer cells, ovarian cancer cells, pancreatic cancer cells, and/or colon cancer cells. In an embodiment, said cancer cells are chronic lymphocytic leukemia cells, breast cancer “HR+” cells, breast cancer “Her2+” cells, breast cancer “triple negative” cells, acute myeloid leukemia cells, non-small cell lung cancer cells, prostate cancer cells, ovarian cancer cells, pancreatic cancer cells, and/or colon cancer cells. A method of treating cancer in a subject in need thereof, and/or enhancing the treatment of cancer in a subject in need thereof, comprising the steps of providing a composition comprising an extract of Terminalia bellerica fruits, and administering said composition in an effective amount to treat and/or enhance treatment of cancer in the subject. In an embodiment, said composition is a standardized aqueous extract of Terminalia bellerica fruits, and said extract is Ayuric®. In an embodiment, said cancer is glioma, breast cancer, chronic lymphocytic leukemia, acute myeloid leukemia, non-small cell lung cancer, prostate cancer cells, ovarian cancer cells, pancreatic cancer cells, and/or colon cancer. In an embodiment, said cancer is chronic lymphocytic leukemia, breast cancer “HR+”, breast cancer “Her2+”, breast cancer “triple negative”, or acute myeloid leukemia.

A method of inhibiting the proliferation of cancer cells comprising the steps of providing a composition comprising an extract of Phyllanthus emblica fruits, and applying said composition to said cancer cells to inhibit proliferation of the cells. The method of claim 17, wherein said composition is a standardized aqueous extract of Phyllanthus emblica fruits, and said extract is Capros®. In an embodiment, said cancer cells are glioma cells, breast cancer cells, chronic lymphocytic leukemia cells, and/or acute myeloid leukemia cells, non-small cell lung cancer cells, prostate cancer cells, ovarian cancer cells, pancreatic cancer cells, and/or colon cancer cells. In an embodiment, said cancer cells are chronic lymphocytic leukemia cells, breast cancer “Her2+” cells, or breast cancer “triple negative” cells. A method of treating cancer in a subject in need thereof and/or enhancing the treatment of cancer in a subject in need thereof, comprising the steps of providing a composition comprising an extract of Phyllanthus emblica fruits, and administering said composition in an effective amount to treat and/or enhance treatment of cancer in the subject. In an embodiment, said composition is a standardized aqueous extract of said Phyllanthus emblica fruits, and said extract is Capros®. In an embodiment, said cancer is glioma, breast cancer, chronic lymphocytic leukemia, acute myeloid leukemia, non-small cell lung cancer, prostate cancer cells, ovarian cancer cells, pancreatic cancer cells, and/or colon cancer. In an embodiment, said cancer is chronic lymphocytic leukemia, breast cancer “Her2+”, or breast cancer “triple negative.”

A method of inhibiting the proliferation of cancer cells comprising the steps of providing a composition comprising an extract of Withania somnifera leaves, roots, or roots plus leaves, and applying said composition to said cancer cells to inhibit proliferation of the cells. In an embodiment, said composition is a standardized aqueous extract or is a standardized hydroalcoholic extract of said leaves, roots, or roots plus leaves, and said extract is Sensoril®. In an embodiment, said cancer cells are glioma cells, breast cancer cells, chronic lymphocytic leukemia cells, acute myeloid leukemia cells including APL cells, non-small cell lung cancer cells, prostate cancer cells, ovarian cancer cells, pancreatic cancer cells, and/or colon cancer cells. In an embodiment, said cancer cells are acute myeloid leukemia cells or breast cancer “triple negative” cells. A method of treating cancer in a subject in need thereof, and/or enhancing the treatment of cancer in a subject in need thereof, comprising the steps of providing a composition comprising an extract of Withania somnifera leaves, roots, or roots plus leaves, and administering said composition in an effective amount to treat and/or enhance treatment of cancer in the subject. In an embodiment, said composition is a standardized aqueous extract or a standardized hydroalcoholic extract of the leaves, roots, or roots plus leaves, and said extract is Sensoril® or Sensoril®-AWE. In an embodiment, said cancer is glioma, breast cancer, chronic lymphocytic leukemia, acute myeloid leukemia including APL, non-small cell lung cancer, prostate cancer, ovarian cancer, pancreatic cancer, and/or colon cancer. In an embodiment, said cancer is acute myeloid leukemia or breast cancer “triple negative”.

A method of inhibiting the proliferation of cancer cells comprising the steps of providing a composition comprising an extract of Shilajit and applying said composition to said cancer cells to inhibit proliferation of the cells. In an embodiment, said extract is a standardized aqueous extract of Shilajit, and said extract is Primavie®. In an embodiment, said cancer cells are glioma cells, breast cancer cells, chronic lymphocytic leukemia cells, acute myeloid leukemia cells, non-small cell lung cancer cells, and/or colon cancer cells. In an embodiment, said cancer cells are acute myeloid leukemia cells. A method of treating cancer in a subject in need thereof, and/or enhancing the treatment of cancer in a subject in need thereof, comprising the steps of providing a composition comprising an extract of Shilajit, and administering said composition in an effective amount to treat and/or enhance treatment of cancer in the subject. In an embodiment, said extract is a standardized aqueous extract of Shilajit, and said extract is Primavie®. In an embodiment, said cancer is glioma, breast cancer, chronic lymphocytic leukemia, acute myeloid leukemia, non-small cell lung cancer, and/or colon cancer. In an embodiment, said cancer is acute myeloid leukemia.

A method of inhibiting the proliferation of cancer cells comprising the steps of providing a composition comprising an extract of Azadirachta indica leaves and twigs, and applying said composition to said cancer cells to inhibit proliferation of the cells. In an embodiment, said composition is a standardized aqueous extract, and said extract is PhytoBGS®. In an embodiment, said cancer cells are glioma cells, breast cancer cells, chronic lymphocytic leukemia cells, acute myeloid leukemia cells, non-small cell lung cancer cells, and/or colon cancer cells. In an embodiment, said cancer cells are acute myeloid leukemia cells or breast cancer “Her2+” cells. A method of treating cancer in a subject in need thereof, and/or enhancing the treatment of cancer in a subject in need thereof, comprising the steps of providing a composition comprising an extract of Azadirachta indica leaves and twigs, and administering said composition in an effective amount to treat and/or enhance treatment of cancer in the subject. In an embodiment, said composition is a standardized aqueous extract, and said extract is PhytoBGS®. In an embodiment, said cancer is glioma, breast cancer, chronic lymphocytic leukemia, acute myeloid leukemia, non-small cell lung cancer, and/or colon cancer. In an embodiment, said cancer is acute myeloid leukemia or breast cancer “Her2+”.

Other embodiments of this invention include a composition comprising a at least one of Terminalia chebula fruits, Terminalia bellerica fruits, Phyllanthus emblica fruits, Withania somnifera roots and leaves, Shilajit, Azadirachta indica leaves and twigs, or a standardized alcohol-water extract of the leaves, roots, or roots plus leaves of Withania somnifera. In an embodiment, said Terminalia chebula fruit extract is AyuFlex®, said Terminalia bellerica fruit extract is Ayuric®, said Phyllanthus emblica fruit extract is Capros®, said Withania somnifera roots and/or leaves extract is Sensoril®, said Shilajit extract is PrimaVie®, and said Azadirachta indica leaves and twigs extract is PhytoBGS®. In an embodiment, said composition comprises a Terminalia chebula fruit extract and a Terminalia bellerica fruit extract, and optionally where said T. chebula extract is AyuFlex® and said T. bellerica extract is Ayuric®. In an embodiment, said composition comprises a Terminalia chebula fruit extract and a Phyllanthus emblica fruit extract, and optionally where said T. chebula extract is AyuFlex® and said P. emblica extract is Capros®. In an embodiment, said composition comprises a Terminalia chebula fruit extract and a Withania somnifera roots and leaves extract, or a standardized alcohol-water extract of the leaves, roots, or roots plus leaves of Withania somnifera, and optionally where said T. chebula extract is AyuFlex® and said W. somnifera extract is Sensoril®. In an embodiment, said composition comprises a Terminalia chebula fruit extract and a Shilajit extract, and optionally where said T. chebula extract is AyuFlex® and said Shilajit extract is PrimaVie®. In an embodiment, said composition comprises a Terminalia chebula fruit extract and an Azadirachta indica leaves and twigs extract, and optionally where said T. chebula extract is AyuFlex® and said A. indica extract is PhytoBGS®. In an embodiment, said composition comprises a Terminalia bellerica fruit extract and a Phyllanthus emblica fruit extract, and optionally where said T. bellerica extract is Ayuric® and said P. emblica extract is Capros®. In an embodiment, said composition comprises a Terminalia bellerica fruit extract and a Withania somnifera roots and/or leaves extract, and optionally where said T. bellerica extract is Ayuric® and said W. somnifera extract is Sensoril®. In an embodiment, said composition comprises a Terminalia bellerica fruit extract and a Shilajit extract, and optionally where said T. bellerica extract is Ayuric® and said Shilajit extract is PrimaVie®. In an embodiment, said composition comprises a Terminalia bellerica fruit extract and an Azadirachta indica leaves and twigs extract, and optionally where said T. bellerica extract is Ayuric® and said A. indica extract is PhytoBGS®. In an embodiment, said composition comprises a Phyllanthus emblica fruit extract and a Withania somnifera roots and/or leaves extract, and optionally where said P. emblica extract is Capros® and said W. somnifera extract is Sensoril®. In an embodiment, said composition comprises a Phyllanthus emblica fruit extract and a Shilajit extract, and optionally where said P. emblica extract is Capros® and said Shilajit extract is PrimaVie®. In an embodiment, said composition comprises a Phyllanthus emblica fruit extract and an Azadirachta indica leaves and twigs extract, and optionally where said P. emblica extract is Capros® and said A. indica extract is PhytoBGS®. In an embodiment, said composition comprises a Withania somnifera roots and leaves extract and a Shilajit extract, and optionally where said W. somnifera extract is Sensoril® and said Shilajit extract is PrimaVie®. In an embodiment, said composition comprises a Withania somnifera roots and/or leaves extract and an Azadirachta indica leaves and twigs extract, and optionally where said W. somnifera extract is Sensoril® and said A. indica extract is PhytoBGS®. In an embodiment, said composition comprises a Shilajit extract and an Azadirachta indica leaves and twigs extract, and optionally where said Shilajit extract is PrimaVie® and said A. indica extract is PhytoBGS®. In an embodiment, said composition comprising Ayuflex® and Ayuric®. In an embodiment, said composition comprising Ayuflex® and Capros®. In an embodiment, said composition comprising Ayuric® and Capros®. In an embodiment, said composition comprising Ayuflex®, Ayuric®, and Capros®. In an embodiment, said composition comprising Sensoril® and PrimaVie®. In an embodiment, said composition comprising Sensoril® and Phyto-BGS®. In an embodiment, said composition comprising PrimaVie® and Phyto-BGS®. In an embodiment, said composition comprising Sensoril®, PrimaVie®, and Phyto-BGS®. In an embodiment, said composition comprises Crominex+3®. In an embodiment, said composition further comprises an anti-cancer drug; in an embodiment, said anti-cancer drug is pictilisib, doxorubicin, temozolomide, docetaxel, 5-fluorouracil (5-FU), ibrutinib, arsenic trioxide, and/or cytarabine, and/or any other anti-cancer drug, preferably identified in this application. In an embodiment, said composition comprises co-administration with an anti-cancer drug. In an embodiment, the present invention is directed to a method of treating cancer in a subject in need thereof, and/or enhancing the treatment of cancer in a subject in need thereof, comprising the steps of providing a composition comprising at least one extract, optionally a standardized aqueous extract that is Ayuflex®, Ayuric®, Capros®, Sensoril®, PrimaVie®, and/or PhytoBGS® or a hydroalcoholic extract that is Sensoril-AWE or a trivalent chromium complex that is Crominex+3®, and administering said composition in combination with an anti-cancer drug in an effective amount to treat and/or enhance treatment of cancer in the subject. In an embodiment, said anti-cancer drug is pictilisib, doxorubicin, temozolomide, docetaxel, 5-fluorouracil (5-FU), ibrutinib, arsenic trioxide, and/or cytarabine, and/or any other anti-cancer drug identified in this application. In an embodiment, a composition of the present invention comprises a combination of an anti-cancer drug, and in a separate composition, an extract. In an embodiment, the present invention is directed to a method of inhibiting the proliferation of cancer cells comprising the steps of providing a composition comprising at least one extract, preferably a standardized aqueous extract, preferably Ayuflex®, Ayuric®, Capros®, Sensoril®, PrimaVie®, and/or PhytoBGS®, and administering said composition in combination with an anti-proliferation drug in an effective amount to inhibit the proliferation of the cancer cells.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±20%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A method of inhibiting the proliferation of cancer cells and/or cancer-associated cells comprising the steps of providing a composition comprising at least one of the group consisting of an extract of Terminalia chebula fruits, an extract of Terminalia bellerica fruits, an extract of Phyllanthus emblica fruits, an extract of Withania somnifera leaves, roots, or roots plus leaves, an extract of Shilajit, an extract of Azadirachta indica leaves and twigs, and a trivalent chromium complex with extracts of Shilajit and P. emblica; and applying said composition to said cancer cells to inhibit proliferation of the cells.

2. The method of claim 1, wherein said extract is a standardized aqueous extract.

3. The method of claim 2, wherein said extract of Terminalia chebula fruits is Ayuflex®, said extract of Terminalia bellerica fruits is Ayuric®, said extract of Phyllanthus emblica fruits is Capros®, said extract of Withania somnifera leaves, roots, or roots plus leaves is Sensoril®, said extract of Shilajit is Primavie®, said extract of Azadirachta indica leaves and twigs is PhytoBGS®, and said trivalent chromium complex with extracts of Shilajit and P. emblica is Crominex+3®.

4. The method of claim 3, wherein said cancer cells are glioma cells, breast cancer cells, chronic lymphocytic leukemia cells, acute myeloid leukemia cells, non-small cell lung cancer cells, small cell lung cancer cells, prostate cancer cells, ovarian cancer cells, pancreatic cancer cells, histiocytic lymphoma cells, and/or colon cancer cells.

5. The method of claim 1, wherein said extract is a standardized hydroalcoholic extract of said Withania somnifera leaves, roots, or roots plus leaves, and said extract is Sensoril-AWE®.

6. The method of claim 5, wherein said cancer cells are glioma cells, breast cancer cells, chronic lymphocytic leukemia cells, acute myeloid leukemia cells, non-small cell lung cancer cells, small cell lung cancer cells, prostate cancer cells, ovarian cancer cells, pancreatic cancer cells, histiocytic lymphoma cells, and/or colon cancer cells.

7. A method of treating cancer in a subject in need thereof, and/or enhancing the treatment of cancer in a subject in need thereof, comprising the steps of providing a composition comprising at least one of the group consisting of an extract of Terminalia chebula fruits, an extract of Terminalia bellerica fruits, an extract of Phyllanthus emblica fruits, an extract of Withania somnifera leaves, roots, or roots plus leaves, an extract of Shilajit, an extract of Azadirachta indica leaves and twigs, and a trivalent chromium complex with extracts of Shilajit and P. emblica; and administering an effective amount of said composition to treat and/or enhance treatment of cancer in the subject.

8. The method of claim 7, wherein said extract is a standardized aqueous extract.

9. The method of claim 8, wherein said extract of Terminalia chebula fruits is Ayuflex®, said extract of Terminalia bellerica fruits is Ayuric®, said extract of Phyllanthus emblica fruits is Capros®, said extract of Withania somnifera leaves, roots, or roots plus leaves is Sensoril®, said extract of Shilajit is Primavie®, said extract of Azadirachta indica leaves and twigs is PhytoBGS®, and said trivalent chromium complex with extracts of Shilajit and P. emblica is Crominex+3®.

10. The method of claim 9, wherein said cancer is glioma, breast cancer, chronic lymphocytic leukemia, acute myeloid leukemia, non-small cell lung cancer, small cell lung cancer, prostate cancer, ovarian cancer, pancreatic cancer, histiocytic lymphoma, and/or colon cancer.

11. The method of claim 7, wherein said extract is a standardized hydroalcoholic extract of said Withania somnifera leaves, roots, or roots plus leaves, and said extract is Sensoril-AWE®.

12. The method of claim 11, wherein said cancer is cancer is glioma, breast cancer, chronic lymphocytic leukemia, acute myeloid leukemia, non-small cell lung cancer, small cell lung cancer, prostate cancer, ovarian cancer, pancreatic cancer, histiocytic lymphoma, and/or colon cancer.

13. A composition comprising at least one of the group consisting of an extract of Terminalia chebula fruits, an extract of Terminalia bellerica fruits, an extract of Phyllanthus emblica fruits, an extract of Withania somnifera leaves, roots, or roots plus leaves, an extract of Shilajit, an extract of Azadirachta indica leaves and twigs, and a trivalent chromium complex with extracts of Shilajit and P. emblica.

14. The composition of claim 13, wherein said composition comprises at least two of the group consisting of an extract of Terminalia chebula fruits, an extract of Terminalia bellerica fruits, an extract of Phyllanthus emblica fruits, an extract of Withania somnifera leaves, roots, or roots plus leaves, an extract of Shilajit, an extract of Azadirachta indica leaves and twigs, and a trivalent chromium complex with extracts of Shilajit and P. emblica.

15. The composition of claim 14, wherein said extract is a standardized aqueous extract, a standardized alcoholic extract, or a standardized hydroalcoholic extract.

16. The composition of claim 14, wherein said composition comprises two or more of the group consisting of Ayuflex®, Ayuric®, Capros®, Sensoril®, Sensoril-AWE®, Primavie®, PhytoBGS®, and Crominex+3®.

17. The composition of claim 16, further comprising an anti-cancer drug.

18. The composition of claim 17, wherein said anti-cancer drug is pictilisib, doxorubicin, temozolomide, docetaxel, 5-fluorouracil (5-FU), ibrutinib, arsenic trioxide, and/or cytarabine.
